(19) United States
(12) Patent Application Publication
(10) Pub. No.: US 2026/0355457 A1
(43) Pub. Date: Nov. 12, 2026

(54) EXPANDABLE SHEATH FOR AN IMPLANT

(71) Applicant: MiRus LLC, Marietta, GA (US)

(72) Inventors: Jay Yadav, Marietta, GA (US); Jason A. White, Marietta, GA (US); Christopher Scott Hayden, Acworth, GA (US)

(21) Appl. No.: 19/650,580

(22) Filed: Apr. 17, 2026

Related U.S. Application Data
(63) Continuation-in-part of application No. 30/003,390, filed on May 13, 2025.
(63) Continuation-in-part of application No. 30/003,485, filed on May 13, 2025.
(60) Provisional application No. 63/803,142, filed on May 9, 2025.
(60) Provisional application No. 63/869,769, filed on Aug. 25, 2025.
(60) Provisional application No. 64/007,976, filed on Mar. 17, 2026.

(51) Int. Cl. A61M 25/00

(52) U.S. Cl. CPC A61M 25/0023; A61M 25/0013; A61M 25/0014; A61M 25/0045; A61M 2025/0024

(57) ABSTRACT

An extendable catheter sheath and an interventional instrument conveying device. The extendable catheter sheath comprises a tube wall (4) which is in a rolling wall structure with a coiled cross section and has an expanding state for expanding the rolling wall structure of a corresponding part and a pre-setting state for automatically recovering the rolling structure. The catheter sheath has a specific cross-section shape, the cross section is changed on the position where an interventional instrument goes by according to the external size of the interventional instrument when the interventional instrument is conveyed, and the catheter sheath is recovered to the original pre-setting state by means of the self-elasticity after the interventional instrument passes by, so that the catheter sheath is favorable for penetrating a thinner blood vessel, the falling of a calcification part is avoided, and the surgical risk is reduced.

### REFERENCED APPLICATIONS

The present application claims priority on U.S. Provisional Patent Application Ser. No. 64/007,976 filed Mar. 17, 2026, which is fully incorporated herein by reference.

The present application claims priority on U.S. Provisional Patent Application Ser. No. 63/803,142 filed May 9, 2025, which is fully incorporated herein by reference.

The present application claims priority on U.S. Provisional Patent Application Ser. No. 63/869,769 filed Aug. 25, 2025, which is fully incorporated herein by reference.

The present application is a continuation-in-part of United States Design Patent Application Ser. No. 30/003,390 filed May 13, 2025, which is fully incorporated herein by reference.

The present application is a continuation-in-part of United States Design Patent Application Ser. No. 30/003,485 filed May 13, 2025, which is fully incorporated herein by reference.

### FIELD OF DISCLOSURE

The present disclosure relates generally to a sheath that is usable with a medical device, particularly to a sheath that is usable with a medical device and which sheath is expandable in at least the region where the medical device is positioned in the sheath, and more particularly to a sheath that is usable with a medical device and which sheath is expandable in at least the region where the medical device is positioned in the sheath and which sheath partially or fully reforms to its same or similar size and shape after the medical device has passed through a portion or all of the sheath. The sheath can be used to protect a body passageway of a patient and/or the incision into the body passageway as a medical device is inserted into and/or through the body passageway and to a treatment site. The sheath can be used with catheter arrangements for stent procedures, angioplasty procedures, embolism procedures, procedures for repairing and/or replacing heart valves (e.g., mitral valves, aortic valve, tricuspid valves, pulmonary or pulmonic valves etc.), procedures for LAA (left atrial appendage) closure devices, etc.

### BACKGROUND

Many modern-day medical devices such as expandable balloons, stents and heart valves are inserted through blood vessels until the medical device reaches a treatment site, and thereafter the medical device is expanded at the treatment site to repair the treatment site and/or to secure the medical device at the treatment site. Many medical devices are formed or include metal materials that can include sharp or abrasive edges or ends. Also, the size of the medical device as it passes into and through a body passageway can cause trauma to the body passageway as a) the medical device and delivery device are inserted into an incision in a patient and into the body passageway, b) the medical device passes through the body passageway to the treatment site, and/or c) the delivery device is removed from the body passageway after delivery of the medical device at the treatment site.

Endovascular delivery catheter assemblies are used to introduce prosthetic devices into a patient and can also be used to deliver the prosthetic device to a treatment area, such as a prosthetic valve or stent, at locations inside the body that are not readily accessible by surgery or where access without invasive surgery is desirable. For example, aortic, mitral, tricuspid, and/or pulmonary prosthetic valves can be delivered to a treatment site using minimally invasive surgical techniques. Transcatheter Aortic Valve Replacement (TAVR) involves delivering a prosthetic aortic valve endovascularly to the native aortic annulus. Access to the patient's vascular system most often utilizes a sheath that serves as a conduit that clinicians use to pass endovascular devices from outside the patient's vasculature into the patient's vasculature.

The sheath is a tubular product that has an open internal lumen that catheters and the like are passed through during a medical procedure. The sheath can be used to introduce a delivery apparatus (e.g., catheter, etc.) into a patient's vasculature. The sheath can be used to minimize damage to a body passageway as the delivery apparatus is inserted through an incision and into a body passageway (e.g., the femoral artery, etc.).

The sheath generally has an elongated sleeve that is inserted into the vasculature and a housing that allows a delivery apparatus to be placed in fluid communication with the vasculature with minimal blood loss. Sheaths that are used in TAVR procedures typically have a first smaller outer diameter when initially inserted into the patient vascular system, which then expands to a larger outer diameter as the TAVR valve passes through the sheath. A conventional TAVR sheath typically requires a tubular loader to be inserted through the seals in the housing to provide an unobstructed path through the housing for a prosthetic valve mounted on the balloon catheter. A conventional loader extends from the proximal end of the expandable sheath. As the prosthetic valve passes through the sheath, the sheath expands and thereafter returns to its smaller outside diameter, thereby reducing injury to the patient's vessel. Typically, the tip of the expandable sheath utilizes a longitudinal cut (through entire thickness of material) through most of the length of the sheath tip, an optionally a small length can remain uncut to hold the expandable tip closed until expansion is desired; however, this is not required. When expansion is desired, a dilator with an outside diameter similar to the largest outside diameter of product can be passed through the sheath and tip, thereby breaking the optional uncut portion of the tip. The dilator is then removed after use, and the sheath can thereafter be used with the delivery catheter to perform the medical procedure.

Non-limiting prior art sheaths are disclosed in U.S. Pat. No. 8,690,936; U.S. Pat. No. 8,790,387; U.S. Pat. No. 9,192,751; U.S. Pat. No. 9,301,840; U.S. Pat. No. 9,301,841; U.S. Pat. No. 9,956,376; U.S. Pat. No. 9,585,743; U.S. Pat. No. 9,801,619; U.S. Pat. No. 9,907,931; U.S. Pat. No. 9,987,134; U.S. Pat. No. 10,631,980; U.S. Pat. No. 10,098,734; U.S. Pat. No. 10,327,896; U.S. Pat. No. 10,391,279; U.S. Pat. No. 10,499,895; U.S. Pat. No. 10,517,720; U.S. Pat. No. 10,524,905; U.S. Pat. No. 10,524,906; U.S. Pat. No. 10,524,907; U.S. Pat. No. 10,548,631; U.S. Pat. No. 10,639,152; U.S. Pat. No. 10,661,049; U.S. Pat. No. 10,792,150; U.S. Pat. No. 10,799,685; U.S. Pat. No. 10,912,919; U.S. Pat. No. 10,918,829; U.S. Pat. No. 10,980,569; U.S. Pat. No. 11,026,719; U.S. Pat. No. 11,045,317, U.S. Pat. No. 11,129,959; U.S. Pat. No. 11,213,318; U.S. Pat. No. 11,273,062; US 11,406,796; U.S. Pat. No. 11,517,432; U.S. Pat. No. 11,540,911; U.S. Pat. No. 11,596,766; U.S. Pat. No. 11,648,115; U.S. Pat. No. 11,786,695; U.S. Pat. No. 11,883,288; U.S. Pat. No. 11,903,829; U.S. Pat. No. 11,931,068; U.S. Pat. No. 11,931,259; U.S. Pat. No. 11,944,770; U.S. Pat. No. 12,011,153; U.S. Pat. No. 12,053,597; U.S. Pat. No. 12,127,960; US 2016/0106562; US 2021/00156612; US 2022/0192827; US 2021/0290376; US 2022/0233310; US 2022/0401216; US 2023/0029387; US 2023/00554898; US 2023/0054952; US 2023/0056245; US 2023/0149674; US 2023/0172715; US 2023/0255768; US 2023/0263991; US 2023/0270546; US 2023/0270547; US 2024/0293229; US 2024/0415648; WO 2023/014551; WO 2023/091413; WO 2023/167811; WO 2023/220121; WO 2024/0225829; WO 2024/107693; WO 2024/205785; WO 2024/263844; all of which are fully incorporated herein by reference.

Conventional methods of accessing a vessel prior to introducing the delivery system include dilating the vessel using multiple dilators or sheaths that progressively increase in diameter. Radially expanding intravascular sheaths tend to have complex mechanisms, such as ratcheting mechanisms that maintain the shaft or sheath in an expanded configuration once a device with a larger diameter than the expandable sheath's original diameter is introduced.

The delivery and/or removal of medical device to or from the body passageway of a patient can expose the patient to risk. Furthermore, accessing a body passageway and inserting the medical device into a body passageway while not damaging the medical device and/or severely damaging the body passageway continues to be a challenge due in part to the relatively large profile of the delivery system that can cause longitudinal and radial tearing of the blood vessel during insertion into the blood vessel.

Also, as catheters are passed through the expandable sheath, it is important for the catheter to remain within the sheath, and not deflect away from the sheath thereby pushing the tip of the sheath away from the catheter. When the catheter deflects away from the sheath tip (not remain coaxial), the sheath tip can be exposed to or otherwise contact the vessel wall and movement of the sheath along the vessel wall can cause damage (dissection, perforation, etc.) to the vessel wall.

Accordingly, there remains a need for an improved sheath for endovascular systems used for implanting stents, prosthetic valves, and other medical devices.

### SUMMARY OF THE DISCLOSURE

The present disclosure relates generally to a sheath that is usable with a medical device, particularly to a sheath that is usable with a medical device and which sheath is expandable in at least the region where the medical device is positioned in the sheath, and more particularly to a sheath that is usable with a medical device and which sheath is expandable in at least the region where the medical device is positioned in the sheath and which sheath partially or fully reforms to its same or similar size and shape after the medical device has passed through a portion or all of the sheath. The sheath can be used to protect a body passageway of a patient and/or the incision into the body passageway as a medical device is inserted into and/or through the body passageway and to a treatment site. The sheath can be used with catheter arrangements for stent procedures, angioplasty procedures, embolism procedures, procedures for repairing and/or replacing heart valves (e.g., mitral valves, aortic valve, tricuspid valves, pulmonary or pulmonic valves etc.), procedures for LAA (left atrial appendage) closure devices, etc.

In one non-limiting aspect of the present disclosure, there is provided an expandable sheath that can be used to reduce or minimize trauma to a body passageway (e.g., blood vessel, duct, etc.) by allowing for temporary expansion of a portion of the expandable sheath to accommodate at least a portion of the delivery system and/or medical device as the medical device and/or delivery system passes through the expandable sheath, followed by a return to the same or similar original diameter or cross-sectional area of the expandable sheath once the delivery system and/or medical device has partially or fully passed through the expandable sheath. In one non-limiting embodiment, the expandable portion of the expandable sheath is configured to expand from a first outer diameter or first outer cross-sectional area to a second outer diameter or second outer cross-sectional area as a medical device (e.g., prosthetic heart valve, stent, angioplasty device, LAA closure device, blood pressure monitoring device, etc.) is moved through an interior passageway in the expandable portion of the expandable sheath, and thereafter the expandable portion of the expandable sheath partially or fully returns to the first outer diameter or first outer cross-sectional area once the medical device has passed partially or fully through the expandable portion of the expandable sheath. The first outer diameter or first outer cross-sectional area is less than the second outer diameter or second outer cross-sectional area. In another and/or alternative non-limiting embodiment, the expandable portion of the expandable sheath can be configured such that the outer diameter or outer cross-sectional area of the expandable sheath and the inner diameter or inner cross-sectional area of the interior passageway progressively expands and contracts as the medical device is moved through the expandable portion of the expandable sheath. In such a configuration, only a portion of the expandable portion of the expandable sheath where the medical device and/or delivery device is located and/or near the location of the medical device and/or delivery device (e.g., 0.1-40 mm within the location of the medical device and all values and ranges therein) in the interior passageway of the expandable sheath expands from the first outer diameter or first outer cross-sectional area to a second outer diameter or second outer cross-sectional area. In the region of the expandable portion of the expandable sheath that is located forwardly and/or rearwardly of where the medical device or delivery device is located in the expandable sheath, the expandable sheath may have an outer diameter or outer cross-sectional area that is the same or similar or greater than the first outer diameter or first outer cross-sectional area, but is less than the second outer diameter or second outer cross-sectional area of the expandable sheath wherein the medical device is located in the expandable portion of the expandable sheath. As such, the expandable portion of the expandable sheath can be configured to progressively increase in outer diameter or cross-sectional area and then progressively decrease in outer diameter or cross-sectional area as the medical device or delivery device moves through the interior passageway of the expandable portion of the expandable sheath. In another and/or alternative non-limiting embodiment, the expandable portion of the expandable sheath can be configured such that the outer diameter or outer cross-sectional area of the expandable sheath and the inner diameter or inner cross-sectional area of the interior passageway expands along the longitudinal length of the expandable sheath as the medical device or delivery device moves through the expandable portion of the expandable sheath, and then partially or fully returns to the first outer diameter or first outer cross-sectional area and first inner diameter or first inner cross-sectional area of the interior passageway once the medical device or delivery partially or fully passes through or is removed from the expandable portion of the expandable sheath. The first inner diameter or first inner cross-sectional area is less than the second inner diameter or second inner cross-sectional area. In another and/or alternative non-limiting embodiment, the expandable portion of the expandable sheath can optionally be partially or fully formed of a shape memory material that facilitates in causing the expandable portion of the expandable sheath to partially or fully return to the first outer diameter or first outer cross-sectional area once the medical device has partially or fully passed through the expandable sheath. In another and/or alternative non-limiting embodiment, the expandable portion of the expandable sheath can be optionally absent a shape memory material, and the expandable portion of the expandable sheath is partially or fully formed of a polymer that has been set in shape in the unexpanded position such that when the polymer is cause to move to an expanded position, the polymer naturally moves partially or fully back to the unexpanded position after the medical device or delivery system has passed, thereby facilitating in causing the expandable portion of the expandable sheath to partially or fully return to the first outer diameter or first outer cross-sectional area once the medical device or delivery system has partially or fully passed through or been removed from the expandable sheath.

In another and/or alternative non-limiting aspect of the present disclosure, the expandable portion of the expandable sheath is partially or fully formed of a polymer material. Non-limiting polymers that can be used include polyether-block-amide (PEBA) (e.g., Pebax™, etc.), polyvinyl acetate or polyvinyl acetate or poly(ethenyl ethanoate) (PVA or PVAC), polytetrafluoroethylene (PTFE), polyimide, polyetheretherketone (PEEK), polyurethane, nylon, polyethylene, polyamide, styrenic block copolymer (SBC) (e.g., polystyrene blocks and rubber blocks [e.g., polybutadiene, polyisoprene or their hydrogenated equivalents]), polyether block amides, polyether block ester copolymer, thermoset silicone, latex, poly-isoprene rubbers, high density polyethylene (HDPE), block copolymers made up of polyamide blocks and polyether blocks (e.g., Pebax™, etc.), polyurethane resin, elastomers formed of block copolymers made up of rigid polyamide blocks and soft polyether blocks, or combinations thereof. In one non-limiting embodiment, the expandable portion of the expandable sheath is formed of 30-100 wt. % (and all values and ranges therebetween) of a polymer material. In one non-limiting specific configuration, 80-100 wt. % of the expandable portion of the expandable sheath is formed of a polymer material. In another and/or alternative non-limiting specific configuration, 30-85 wt. % of the expandable portion of the expandable sheath is formed of a polymer material. The expandable portion of the expandable sheath can be formed of one or more different polymers.

In another and/or alternative non-limiting aspect of the present disclosure, the expandable portion of the expandable sheath is partially or fully formed of a polymer material and wherein the expandable portion of the expandable sheath includes one or more polymer portions. In one non-limiting embodiment, the expandable portion of the sheath is partially or fully formed of a single polymer portion. The single polymer portion can be formed of one or more different polymers and/or be formed of one or more polymer layers. In another and/or alternative non-limiting embodiment, the expandable portion of the sheath is partially or fully formed of two polymer portions, wherein the first polymer portion constitutes 2-98 wt. % (and all values and ranges therebetween) of the total polymer in the expandable sheath, and the second polymer portion constitutes 2-98 wt. % (and all values and ranges therebetween) of the total polymer in the expandable sheath. The first and/or second polymer portions can be formed of one or more different polymers and/or be formed of one or more polymer layers. In another and/or alternative non-limiting embodiment, the expandable portion of the sheath is partially or fully formed of three polymer portions, and wherein the first polymer portion constitutes 2-96 wt. % (and all values and ranges therebetween) of the total polymer in the expandable sheath, the second polymer portion constitutes 2-96 wt. % (and all values and ranges therebetween) of the total polymer in the expandable sheath, and the third polymer portion constitutes 2-96 wt. % (and all values and ranges therebetween) of the total polymer in the expandable sheath. The first, second and/or third polymer portions can be formed of one or more different polymers and/or be formed of one or more polymer layers.

In another and/or alternative non-limiting aspect of the present disclosure, the expandable portion of the expandable sheath is partially or fully formed of a polymer material and wherein the expandable portion of the expandable sheath is partially or fully formed of a first polymer portion, and wherein the first polymer portion forms an outer layer of the expandable portion of the expandable sheath, and the first polymer portion forms a continuous outer surface of the sheath, and wherein the first polymer portion is optionally not a stretchable material (e.g., a stretchable material is a material that can be stretched in length and after the stretching force is removed from the material, the stretchable material returns to its original shape and 90-100% of the original length (and all values and ranges therebetween) after being stretched [e.g., elastomeric material]), and wherein the first polymer portion has at least first and second thicknesses about an outer circumference of the first polymer portion, and wherein when the expandable portion of the sheath is in the expanded orientation then 55-98% (and all values and ranges therebetween) of the outer circumference of the first polymer portion has a first thickness, and 2-45% (and all values and ranges therebetween) of the outer circumference of the first polymer portion has a second thickness, and wherein the first thickness is greater than the second thickness, and wherein the portion of the first polymer portion that has the second thickness is optionally foldable (e.g., a foldable portion is a portion wherein 30-100% of a longitudinal length (and all values and ranges therebetween) of the portion can be folded 1-100 times [and all values and ranges therebetween] from a flat planar orientation to an orientation wherein one region of the foldable portion remains in the planar orientation and another region is folded to at least 60° [e.g., 60-180° and all values and ranges therebetween] and then unfolded back to the original planar orientation without permanently damaging the foldable portion), and wherein the portion of the first polymer portion that has the first thickness is optionally not foldable, and wherein the portion of the first polymer portion that has the first thickness is bendable or flexible between the expanded and unexpanded orientation of the expandable sheath, and wherein the portion of the first polymer portion that has the first thickness is optionally configured to flex or bend back to 80-100% (and all values and ranges therebetween) of the original unexpanded orientation of the expandable sheath after the expandable sheath has been expanded to the expanded orientation and the force that caused the expandable sheath to expand to the expanded orientation (e.g., expansion force by a medical device moving through the internal cavity of the expandable sheath, expansion force by a medical instrument moving through the internal cavity of the expandable sheath, expansion force by a medical delivery device moving through the internal cavity of the expandable sheath, etc.) has been removed from the expandable sheath. In one non-limiting embodiment, the first polymer portion is formed of polyvinyl acetate or poly(ethenyl ethanoate); however, other polymers can be used. In another and/or alternative non-limiting embodiment, the durometer of the first polymer portion is 20-90 (Shore D scale) and all values and ranges therebetween. In another and/or alternative non-limiting embodiment, the average thickness of the first thickness is 0.002-0.03 inches (and all values and ranges therebetween), and the average thickness of the second thickness is 0.001-0.01 inches (and all values and ranges therebetween). In one non-limiting configuration, the average first thickness is 0.003-0.008 inches, and the average second thickness is 0.001-0.0028 inches. In another and/or alternative non-limiting embodiment, the portion of the first polymer portion that has the second thickness is optionally configured to partially fold when the expandable portion of the sheath is in the unexpanded orientation and partially unfold when the expandable portion of the sheath is in the expanded orientation, and wherein 30-99.9% (and all values and ranges therebetween) of the longitudinal length of the first polymer portion that has the second thickness is positioned between overlapping regions of the first polymer portion that has the first thickness when the expandable portion of the sheath is in the fully unexpanded position or orientation. In another and/or alternative non-limiting embodiment, the portion of the first polymer portion that has the second thickness is optionally stretchable. The first polymer portion can be formed of one or more different polymers and/or be formed of one or more polymer layers.

In another and/or alternative non-limiting aspect of the present disclosure, the expandable portion of the expandable sheath is partially or fully formed of a polymer material and wherein the expandable portion of the expandable sheath is partially or fully formed of a first polymer portion having a first thickness, and a second polymer portion that has a second thickness, and wherein the first thickness is optionally greater than the second thickness, and wherein one of the ends of the second polymer portion is connected to one end of the first polymer portion, and the other end of the second polymer portion is connected to the other end of the first polymer portion, and wherein the outer surface of the first and second polymer portions forms a continuous outer surface of the expandable sheath. In one non-limiting embodiment, the first polymer portion and/or the second polymer portion is optionally not a stretchable material. In another and/or alternative non-limiting embodiment, the first polymer portion is not a stretchable material, and the second polymer portion is a stretchable material. In another and/or alternative non-limiting embodiment, when the expandable portion of the sheath is in the expanded orientation, and the first and second polymer portion form the outer surface of the sheath, the first polymer portion then extends 55-98% (and all values and ranges therebetween) about the outer perimeter of the sheath, and the second polymer portion then extends 2-45% (and all values and ranges therebetween) about the outer perimeter of the sheath. In another and/or alternative non-limiting embodiment, the first polymer portion has a thickness and is formed of a polymer material that enables the first polymer portion to flex or bend between a contracted and expanded orientation; however, the first polymer portion is optionally generally not a foldable portion (e.g., a foldable portion is a portion wherein 30-100% of a longitudinal length (and all values and ranges therebetween) of the portion can be folded 1-100 times [and all values and ranges therebetween] from a flat planar orientation to an orientation wherein one region of the foldable portion remains in the planar orientation and another region is folded to at least 60° [e.g., 60-180° and all values and ranges therebetween] and then unfolded back to the original planar orientation without permanently damaging the foldable layer). In another and/or alternative non-limiting embodiment, the second polymer portion has a thickness and is formed of a polymer material that enables the second polymer portion to be flexible between a contracted and expanded orientation, and also allows the second polymer portion to be optionally foldable. In another and/or alternative non-limiting embodiment, the first polymer portion is optionally not foldable, and the first polymer portion is bendable or flexible between the expanded and unexpanded orientation of the expandable sheath, and wherein the first polymer portion is optionally configured to flex or bend back to 80-100% (and all values and ranges therebetween) of the original unexpanded orientation of the expandable sheath after the expandable sheath has been expanded to the expanded orientation and the force that caused the expandable sheath to expand to the expanded orientation (e.g., expansion force by a medical device moving through the internal cavity of the expandable sheath, expansion force by a medical instrument moving through the internal cavity of the expandable sheath, expansion force by a medical delivery device moving through the internal cavity of the expandable sheath, etc.) has been removed from the expandable sheath. In one non-limiting embodiment, the first and/or second polymer portions are formed of polyvinyl acetate or poly(ethenyl ethanoate); however, other polymers can be used. In another and/or alternative non-limiting embodiment, the durometer of the first polymer portion is 20-90 (Shore D scale) and all values and ranges therebetween. In one non-limiting configuration, the durometer of the first polymer portion is greater than the durometer of the second polymer portion. In another and/or alternative non-limiting embodiment, the average thickness of the first polymer portion is 0.002-0.03 inches (and all values and ranges therebetween), and the average thickness of the second polymer portion is 0.001-0.01 inches (and all values and ranges therebetween). In one non-limiting configuration, the average thickness of the first polymer portion is 0.003-0.008 inches, and the average thickness of the second polymer portion is 0.001-0.0028 inches. In one non-limiting configuration, the average thickness of the first polymer portion is greater than the average thickness of the second polymer portion. In another and/or alternative non-limiting embodiment, one or both ends of the second polymer portion is connected to ends of the first polymer portion by heat bonding, adhesive, mechanical connection [e.g., snap connection, clip connection, staple connection, pin connection, stitching, friction fit connection, etc.), melt bonding, welded bond, use of bonding layer or bonding strips, etc. As can be appreciated, two or more bonding arrangements can be used (e.g., adhesive and heat bonding, heat bonding and use of bonding layer such as an inner and/or outer polymer coating, etc.). In another and/or alternative non-limiting embodiment, the portion of the second polymer portion is optionally configured to partially fold when the expandable portion of the sheath is in the unexpanded orientation and partially unfold when the expandable portion of the sheath is in the expanded orientation, and wherein 30-99.9% (and all values and ranges therebetween) of the longitudinal length of the second polymer portion is positioned between overlapping regions of the first polymer portion when the expandable portion of the expandable sheath is in the fully unexpanded position or orientation. The first and/or second polymer portions can be formed of one or more different polymers and/or be formed of one or more polymer layers.

In another and/or alternative non-limiting aspect of the present disclosure, the expandable portion of the expandable sheath is partially or fully formed of a polymer material and wherein the expandable portion of the expandable sheath is partially or fully formed of first and second polymer portions, and wherein a) the first polymer portion forms an outer layer of the expandable portion of the expandable sheath, and the first polymer portion forms a continuous outer layer, and wherein the first polymer portion is optionally not a stretchable material, and wherein the first polymer portion optionally has a constant thickness along 50-100% (and all values and ranges therebetween) of the longitudinal length and/or circumference of the first polymer portion, and wherein at least a portion (e.g., 10-100% and all values and ranges therebetween) of the longitudinal length of the first polymer portion is optionally foldable, and b) the second polymer portion is a non-continuous layer that has two ends that are not connected together, and wherein the two ends are optionally separated from one another when the expandable portion of the expandable sheath is in the expanded orientation, and wherein the second polymer portion is formed of a same or different material from the first polymer portion, and wherein a durometer and/or thickness of the second polymer portion is optionally greater than a durometer and/or thickness of the first polymer portion, and wherein the second polymer portion is optionally connected (e.g., adhesive connection, heat flow or melted connection, welded connection, mechanical connection, etc.) to a portion of a bottom surface of the first polymer portion (e.g., 0.1-95% of the longitudinal length of the bottom surface and all values and ranges therebetween), and wherein the portion of the first polymer portion that is not optionally connected to the second polymer portion is optionally foldable when the second polymer portion is in the non-expanded orientation, and wherein the average thickness of the second polymer portion is optionally greater than the average thickness of the first polymer portion. In one non-limiting embodiment, the first and/or second polymer portions are formed of polyvinyl acetate or poly(ethenyl ethanoate); however, other polymers can be used. In another and/or alternative non-limiting embodiment, the durometer of the first and second polymer portions is 20-90 (Shore D scale) and all values and ranges therebetween. The durometer of the first and second polymer portions can be the same or different. In one non-limiting configuration, the durometer of the first polymer portion is less than the durometer of the second polymer portion. In another and/or alternative non-limiting embodiment, the average thickness of the second polymer portion is 0.002-0.01 inches (and all values and ranges therebetween), and the average thickness of the first polymer portion is 0.001-0.003 inches (and all values and ranges therebetween). In one non-limiting configuration, the first polymer portion has a thickness of 0.001-0.0035 inches, the second polymer portion has an average thickness is 0.003-0.008 inches. In one non-limiting configuration, the average thickness of the second polymer portion is greater than the average thickness of the first polymer portion. The outer surface of the second polymer portion and/or the bottom surface of the first polymer portion can optionally be etched or otherwise roughened to facilitate in the optional connection of the second polymer portion to the first polymer portion. In another and/or alternative non-limiting embodiment, the portion of the first polymer portion is optionally configured to partially fold when the expandable portion of the sheath is in the unexpanded orientation and partially unfold when the expandable portion of the sheath is in the expanded orientation, and wherein 30-99.9% (and all values and ranges therebetween) of the longitudinal length of the first polymer portion is positioned between overlapping regions of the second polymer portion when the expandable portion of the sheath is in the fully unexpanded position or orientation. In another and/or alternative non-limiting embodiment, the second polymer portion has a thickness and is formed of a polymer material that enables the second polymer portion to be flexible between a contracted and expanded orientation. In another and/or alternative non-limiting embodiment, the second polymer portion is optionally not foldable, and the second polymer portion is bendable or flexible between the expanded and unexpanded orientation of the expandable sheath, and wherein the second polymer portion is optionally configured to flex or bend back to 80-100% (and all values and ranges therebetween) of the original unexpanded orientation of the expandable sheath after the expandable sheath has been expanded to the expanded orientation and the force that caused the expandable sheath to expand to the expanded orientation (e.g., expansion force by a medical device moving through the internal cavity of the expandable sheath, expansion force by a medical instrument moving through the internal cavity of the expandable sheath, expansion force by a medical delivery device moving through the internal cavity of the expandable sheath, etc.) has been removed from the expandable sheath. The first and/or second polymer portions can be formed of one or more different polymers and/or be formed of one or more polymer layers.

In another and/or alternative non-limiting aspect of the present disclosure, the expandable portion of the expandable sheath is partially or fully formed of a polymer material and wherein the expandable portion of the expandable sheath is partially or fully formed of a first polymer portion and a second polymer portion, and wherein the first polymer portion forms an outer layer of the expandable portion of the expandable sheath, and the first polymer portion forms a continuous outer surface of the sheath, and wherein the first polymer portion encircles the second polymer portion, and wherein 1-100% (and all values and ranges therebetween) of the top surface of the second polymer portion is optionally connected to a bottom surface of the first polymer portion, and wherein the first polymer portion is optionally not a stretchable material (e.g., a stretchable material is a material that can be stretched in length and after the stretching force is removed from the material, the stretchable material returns to its original shape and 90-100% of the original length (and all values and ranges therebetween) after being stretched [e.g., elastomeric material]), and wherein the first polymer portion has at least first and second thicknesses about an outer circumference of the first polymer portion, and wherein when the expandable portion of the sheath is in the expanded orientation then 55-98% (and all values and ranges therebetween) of the outer circumference of the first polymer portion has a first thickness, and 2-45% (and all values and ranges therebetween) of the outer circumference of the first polymer portion has a second thickness, and wherein the first thickness is greater than the second thickness, and wherein the portion of the first polymer portion that has the second thickness is optionally foldable (e.g., a foldable portion is a portion wherein 30-100% of a longitudinal length (and all values and ranges therebetween) of the portion can be folded 1-100 times [and all values and ranges therebetween] from a flat planar orientation to an orientation wherein one region of the foldable portion remains in the planar orientation and another region is folded to at least 60° [e.g., 60-180° and all values and ranges therebetween] and then unfolded back to the original planar orientation without permanently damaging the foldable portion), and wherein the portion of the first polymer portion that has the first thickness is optionally not foldable, and wherein the portion of the first polymer portion that has the first thickness is bendable or flexible between the expanded and unexpanded orientation of the expandable sheath, and wherein the portion of the first polymer portion that has the first thickness is optionally configured to flex or bend back to 80-100% (and all values and ranges therebetween) of the original unexpanded orientation of the expandable sheath after the expandable sheath has been expanded to the expanded orientation and the force that caused the expandable sheath to expand to the expanded orientation (e.g., expansion force by a medical device moving through the internal cavity of the expandable sheath, expansion force by a medical instrument moving through the internal cavity of the expandable sheath, expansion force by a medical delivery device moving through the internal cavity of the expandable sheath, etc.) has been removed from the expandable sheath. In one non-limiting embodiment, the second polymer portion is optionally a continuous layer, and wherein the second polymer portion is formed of a different material from the first polymer portion, and wherein a durometer and/or thickness of the second polymer portion is optionally different from a durometer and/or thickness of the first polymer portion, and wherein the second polymer portion is optionally connected (e.g., adhesive connection, heat flow or melted connection, welded connection, mechanical connection, etc.) to a portion or all of a bottom surface of the first polymer portion, and wherein the portion of the second polymer portion that is optionally connected to the thinner portion of the first polymer portion is optionally foldable with the thinner portion of the first polymer portion when the expandable portion of the expandable sheath is in the non-expanded orientation. In one non-limiting configuration, the average thickness of the second polymer portion is less than the average thickness of the thicker portion of the first pol thicker portion of the first polymer portion. In another and/or alternative non-limiting embodiment, the first polymer portion is formed of polyvinyl acetate or poly(ethenyl ethanoate); however, other polymers can be used. In another and/or alternative non-limiting embodiment, the second polymer portion is formed of polytetrafluoroethylene (PTFE); however, other polymers can be used. In another and/or alternative non-limiting embodiment, the durometer of the first polymer portion is 20-90 (Shore D scale) and all values and ranges therebetween. In another and/or alternative non-limiting embodiment, the durometer of the second polymer portion is 50-65 (Shore D scale) and all values and ranges therebetween. The durometer of the first and second polymer portions can be the same or different. In one non-limiting configuration, the durometer of the first polymer portion is greater than the durometer of the second polymer portion. In another and/or alternative non-limiting embodiment, the average thickness of the first polymer ranges from 0.001-0.01 inches (and all values and ranges therebetween). In another and/or alternative non-limiting embodiment, the average thickness of the first thickness is 0.002-0.03 inches (and all values and ranges therebetween), and the average thickness of the second thickness is 0.001-0.01 inches (and all values and ranges therebetween). In one non-limiting configuration, the average first thickness is 0.003-0.008 inches, and the average second thickness is 0.001-0.0028 inches. In another and/or alternative non-limiting embodiment, the average thickness of the second polymer portion is 0.001-0.008 inches (and all values and ranges therebetween). In another and/or alternative non-limiting embodiment, the portion of the first polymer portion that has the second thickness is optionally configured to partially fold when the expandable portion of the sheath is in the unexpanded orientation and partially unfold when the expandable portion of the sheath is in the expanded orientation, and wherein 30-99.9% (and all values and ranges therebetween) of the longitudinal length of the first polymer portion that has the second thickness is positioned between overlapping regions of the first polymer portion that has the first thickness when the expandable portion of the sheath is in the fully unexpanded position or orientation. In another and/or alternative non-limiting embodiment, the portion of the first polymer portion that has the second thickness is optionally stretchable; however, this is not required. The top surface of the second polymer portion and/or bottom surface of the first polymer portion can optionally be etched or otherwise roughened to facilitate in the connection of the second polymer portion to one or more regions of the first polymer portion. In another and/or alternative non-limiting embodiment, at least a portion of the second polymer portion is optionally configured to partially fold when the expandable portion of the sheath is in the unexpanded orientation and partially unfold when the expandable portion of the sheath is in the expanded orientation, and wherein 1-60% (and all values and ranges therebetween) of the longitudinal length and/or circumference of the second polymer portion is positioned between overlapping regions of the first polymer portion that have the first thickness when the expandable portion of the sheath is in the fully unexpanded position or orientation. The first and/or second polymer portions can be formed of one or more different polymers and/or be formed of one or more polymer layers.

In another and/or alternative non-limiting aspect of the present disclosure, the expandable portion of the expandable sheath is partially or fully formed of a polymer material and wherein the expandable portion of the expandable sheath is partially or fully formed of three polymer portions. In one non-limiting embodiment, the first polymer portion has a first thickness, and the second polymer portion that has a second thickness, and wherein the first thickness is optionally greater than the second thickness, and wherein one of the ends of the second polymer portion is connected to one end of the first polymer portion, and the other end of the second polymer portion is connected to the other end of the first polymer portion, and wherein the outer surface of the first and second polymer portions forms a continuous outer surface of the expandable sheath, and wherein the third polymer portion is encircled by the first and second polymer portions. In another and/or alternative non-limiting embodiment, the first polymer portion and/or the second polymer portion is optionally not a stretchable material. In another and/or alternative non-limiting embodiment, the first polymer portion is not a stretchable material, and the second polymer portion is a stretchable material. In another and/or alternative non-limiting embodiment, when the expandable portion of the sheath is in the expanded orientation, and the first and second polymer portion form the outer surface of the sheath, the first polymer portion then extends 55-98% (and all values and ranges therebetween) about the outer perimeter of the sheath, and the second polymer portion then extends 2-45% (and all values and ranges therebetween) about the outer perimeter of the sheath. In another and/or alternative non-limiting embodiment, the first polymer portion has a thickness and is formed of a polymer material that enables the first polymer portion to flex or bend between a contracted and expanded orientation; however, the first polymer portion is optionally generally not a foldable portion (e.g., a foldable portion is a portion wherein 30-100% of a longitudinal length (and all values and ranges therebetween) of the portion can be folded 1-100 times [and all values and ranges therebetween] from a flat planar orientation to an orientation wherein one region of the foldable portion remains in the planar orientation and another region is folded to at least 60° [e.g., 60-180° and all values and ranges therebetween] and then unfolded back to the original planar orientation without permanently damaging the foldable layer). In another and/or alternative non-limiting embodiment, the second polymer portion has a thickness and is formed of a polymer material that enables the second polymer portion to be flexible between a contracted and expanded orientation, and also allows the second polymer portion to be optionally foldable. In another and/or alternative non-limiting embodiment, the first polymer portion is optionally not foldable, and the first polymer portion is bendable or flexible between the expanded and unexpanded orientation of the expandable sheath, and wherein the first polymer portion is optionally configured to flex or bend back to 80-100% (and all values and ranges therebetween) of the original unexpanded orientation of the expandable sheath after the expandable sheath has been expanded to the expanded orientation and the force that caused the expandable sheath to expand to the expanded orientation (e.g., expansion force by a medical device moving through the internal cavity of the expandable sheath, expansion force by a medical instrument moving through the internal cavity of the expandable sheath, expansion force by a medical delivery device moving through the internal cavity of the expandable sheath, etc.) has been removed from the expandable sheath. In one non-limiting embodiment, the first and/or second polymer portions are formed of polyvinyl acetate or poly(ethenyl ethanoate); however, other polymers can be used. In another and/or alternative non-limiting embodiment, the durometer of the first polymer portion is 20-90 (Shore D scale) and all values and ranges therebetween. In another and/or alternative non-limiting embodiment, one or both ends of the second polymer portion is connected to ends of the first polymer portion by heat bonding, adhesive, mechanical connection [e.g., snap connection, clip connection, staple connection, pin connection, stitching, friction fit connection, etc.), melt bonding, welded bond, use of bonding layer or bonding strips, etc. As can be appreciated, two or more bonding arrangements can be used (e.g., adhesive and heat bonding, heat bonding and use of bonding layer such as an inner and/or outer polymer coating, etc.). In another and/or alternative non-limiting embodiment, the portion of the second polymer portion is optionally configured to partially fold when the expandable portion of the sheath is in the unexpanded orientation and partially unfold when the expandable portion of the sheath is in the expanded orientation, and wherein 30-99.9% (and all values and ranges therebetween) of the longitudinal length of the second polymer portion is positioned between overlapping regions of the first polymer portion when the expandable portion of the expandable sheath is in the fully unexpanded position or orientation. In another and/or alternative non-limiting embodiment, the third polymer portion is optionally a continuous layer, and wherein the third polymer portion is formed of a different material from the first and/or second polymer portions, and wherein a durometer and/or thickness of the third polymer portion is optionally different from a durometer and/or thickness of the first and/or second polymer portions, and wherein the third polymer portion is optionally connected (e.g., adhesive connection, heat flow or melted connection, welded connection, mechanical connection, etc.) to a portion or all of a bottom surface of the first and/or second polymer portions, and wherein the portion of the third polymer portion that is optionally connected to the second polymer portion is optionally foldable with the second polymer portion when the expandable portion of the expandable sheath is in the non-expanded orientation. In another and/or alternative non-limiting embodiment, the first and/or second polymer portions is formed of polyvinyl acetate or poly(ethenyl ethanoate); however, other polymers can be used. In another and/or alternative non-limiting embodiment, the third polymer portion is formed of polytetrafluoroethylene (PTFE); however, other polymers can be used. In another and/or alternative non-limiting embodiment, the durometer of the first and/or second polymer portions is 20-90 (Shore D scale) and all values and ranges therebetween. In another and/or alternative non-limiting embodiment, the durometer of the third polymer portion is 50-65 (Shore D scale) and all values and ranges therebetween. The durometer of the first, second and third polymer portions can be the same or different. In one non-limiting configuration, the durometer of the first polymer portion is greater than the durometer of the third polymer portion. In another and/or alternative non-limiting configuration, the durometer of the second polymer portion is less than the durometer of the third polymer portion. In another and/or alternative non-limiting embodiment, the average thickness of the first polymer portion is 0.002-0.03 inches (and all values and ranges therebetween), and the average thickness of the second polymer portion is 0.001-0.01 inches (and all values and ranges therebetween). In one non-limiting configuration, the average thickness of the first polymer portion is 0.003-0.008 inches, and the average thickness of the second polymer portion is 0.001-0.0028 inches. In another and/or alternative non-limiting embodiment, the average thickness of the second polymer portion is 0.001-0.008 inches (and all values and ranges therebetween). In another and/or alternative non-limiting embodiment, the average thickness of the third polymer portion is 0.002-0.01 inches (and all values and ranges therebetween). The top surface of the third polymer portion and/or bottom surface of the first and/or second polymer portions can optionally be etched or otherwise roughened to facilitate in the connection of the third polymer portion to one or more regions of the first and/or second polymer portions. In another and/or alternative non-limiting embodiment, at least a portion of the third polymer portion is optionally configured to partially fold when the expandable portion of the sheath is in the unexpanded orientation and partially unfold when the expandable portion of the sheath is in the expanded orientation, and wherein 1-60% (and all values and ranges therebetween) of the longitudinal length and/or circumference of the third polymer portion is positioned between overlapping regions of the first polymer portion when the expandable portion of the sheath is in the fully unexpanded position or orientation. The first, second and/or third polymer portions can be formed of one or more different polymers and/or be formed of one or more polymer layers.

In another and/or alternative non-limiting aspect of the present disclosure, the expandable portion of the expandable sheath is partially or fully formed of a polymer material and wherein the expandable portion of the expandable sheath is partially or fully formed of three polymer portions. In one non-limiting embodiment, a) the first polymer portion forms an outer layer of the expandable portion of the expandable sheath, and the first polymer portion forms a continuous outer layer, and wherein the first polymer portion is optionally not a stretchable material, and wherein the first polymer portion optionally has a constant thickness along 50-100% (and all values and ranges therebetween) of the longitudinal length and/or circumference of the first polymer portion, and wherein at least a portion (e.g., 10-100% and all values and ranges therebetween) of the longitudinal length of the first polymer portion is optionally foldable, and b) the second polymer portion is a non-continuous layer that has two ends that are not connected together, and wherein the two ends are optionally separated from one another when the expandable portion of the expandable sheath is in the expanded orientation, and wherein the second polymer portion is formed of a same or different material from the first polymer portion, and wherein a durometer and/or thickness of the second polymer portion is optionally greater than a durometer and/or thickness of the first polymer portion, and wherein the second polymer portion is optionally connected (e.g., adhesive connection, heat flow or melted connection, welded connection, mechanical connection, etc.) to a portion of a bottom surface of the first polymer portion (e.g., 0.1-95% of the longitudinal length of the bottom surface and all values and ranges therebetween), and wherein the portion of the first polymer portion that is not optionally connected to the second polymer portion is optionally foldable when the second polymer portion is in the non-expanded orientation. In one non-limiting embodiment, the first and/or second polymer portions are formed of polyvinyl acetate or poly(ethenyl ethanoate); however, other polymers can be used. In another and/or alternative non-limiting embodiment, the durometer of the first and second polymer portions is 20-90 (Shore D scale) and all values and ranges therebetween. The durometer of the first and second polymer portions can be the same or different. In one non-limiting configuration, the durometer of the first polymer portion is less than the durometer of the second polymer portion. In another and/or alternative non-limiting embodiment, the average thickness of the second polymer portion is 0.002-0.01 inches (and all values and ranges therebetween), and the average thickness of the first polymer portion is 0.001-0.003 inches (and all values and ranges therebetween). In one non-limiting configuration, the first polymer portion has a thickness of 0.001-0.0035 inches, the second polymer portion has an average thickness is 0.003-0.008 inches. The outer surface of the second polymer portion and/or the bottom surface of the first polymer portion can optionally be etched or otherwise roughened to facilitate in the optional connection of the second polymer portion to the first polymer portion. In another and/or alternative non-limiting embodiment, the portion of the first polymer portion is optionally configured to partially fold when the expandable portion of the sheath is in the unexpanded orientation and partially unfold when the expandable portion of the sheath is in the expanded orientation, and wherein 30-99.9% (and all values and ranges therebetween) of the longitudinal length of the first polymer portion is positioned between overlapping regions of the second polymer portion when the expandable portion of the sheath is in the fully unexpanded position or orientation. In another and/or alternative non-limiting embodiment, the second polymer portion has a thickness and is formed of a polymer material that enables the second polymer portion to be flexible between a contracted and expanded orientation. In another and/or alternative non-limiting embodiment, the second polymer portion is optionally not foldable, and the second polymer portion is bendable or flexible between the expanded and unexpanded orientation of the expandable sheath, and wherein the second polymer portion is optionally configured to flex or bend back to 80-100% (and all values and ranges therebetween) of the original unexpanded orientation of the expandable sheath after the expandable sheath has been expanded to the expanded orientation and the force that caused the expandable sheath to expand to the expanded orientation (e.g., expansion force by a medical device moving through the internal cavity of the expandable sheath, expansion force by a medical instrument moving through the internal cavity of the expandable sheath, expansion force by a medical delivery device moving through the internal cavity of the expandable sheath, etc.) has been removed from the expandable sheath. In another and/or alternative non-limiting embodiment, the third polymer portion is optionally a continuous layer, and wherein the third polymer portion is formed of a different material from the first and/or second polymer portions, and wherein a durometer and/or thickness of the third polymer portion is optionally different from a durometer and/or thickness of the first and/or second polymer portions, and wherein the third polymer portion is optionally connected (e.g., adhesive connection, heat flow or melted connection, welded connection, mechanical connection, etc.) to a portion or all of a bottom surface of the first and/or second polymer portions, and wherein the portion of the third polymer portion that is optionally connected to the first polymer portion is optionally foldable with the first polymer portion when the expandable portion of the expandable sheath is in the non-expanded orientation. In another and/or alternative non-limiting embodiment, the third polymer portion is formed of polytetrafluoroethylene (PTFE); however, other polymers can be used. In another and/or alternative non-limiting embodiment, the durometer of the third polymer portion is 50-65 (Shore D scale) and all values and ranges therebetween. The durometer of the first, second and third polymer portions can be the same or different. In one non-limiting configuration, the durometer of the second polymer portion is greater than the durometer of the third polymer portion. In another and/or alternative non-limiting configuration, the durometer of the first polymer portion is less than the durometer of the third polymer portion. In another and/or alternative non-limiting embodiment, the average thickness of the third polymer portion is 0.002-0.01 inches (and all values and ranges therebetween). The top surface of the third polymer portion and/or bottom surface of the first and/or second polymer portions can optionally be etched or otherwise roughened to facilitate in the connection of the third polymer portion to one or more regions of the first and/or second polymer portions. In another and/or alternative non-limiting embodiment, at least a portion of the third polymer portion is optionally configured to partially fold when the expandable portion of the sheath is in the unexpanded orientation and partially unfold when the expandable portion of the sheath is in the expanded orientation, and wherein 1-60% (and all values and ranges therebetween) of the longitudinal length and/or circumference of the third polymer portion is positioned between overlapping regions of the second polymer portion when the expandable portion of the sheath is in the fully unexpanded position or orientation. The first, second and/or third polymer portions can be formed of one or more different polymers and/or be formed of one or more polymer layers.

In another and/or alternative non-limiting aspect of the present disclosure, the expandable portion of the expandable sheath optionally includes a stretchable outer polymer layer that a) forms a continuous outer layer of at least a portion of the expandable portion of the expandable sheath, b) extends 1-100% (and all values and ranges therebetween) of the longitudinal length of the expandable portion of the expandable sheath, and/or c) optionally has a constant thickness about 50-100% (and all values and ranges therebetween) of the outer perimeter and/or longitudinal length of the outer polymer layer. The stretchable outer polymer layer can have the same or different thickness and/or durometer to one or more of the other polymer portions of the expandable sheath. In one non-limiting embodiment, the stretchable outer polymer layer is optionally formed of a different material from the material used to form the other polymer portions of the expandable sheath. In another and/or alternative non-limiting embodiment, the stretchable outer polymer layer is optionally the only stretchable polymer layer or polymer portion of the expandable portions of the expandable sheath. In another and/or alternative non-limiting embodiment, the stretchable outer polymer layer is optionally connected (e.g., adhesive connection, heat flow or melted connection, welded connection, mechanical connection, etc.) to a portion or all of a bottom surface of one or more other polymer portions of the expandable sheath. In another and/or alternative non-limiting embodiment, the stretchable outer polymer layer is optionally foldable. In one non-limiting configuration, the stretchable outer polymer layer is optionally foldable such that at least a portion of the stretchable outer polymer layer folds when the expandable portion of the sheath is in the unexpanded orientation and partially unfolds when the expandable portion of the sheath is in the expanded orientation, and wherein 30-99.9% (and all values and ranges therebetween) of the longitudinal length and/or circumference of the stretchable outer polymer layer is positioned between overlapping regions of the one or more polymer portions of the expandable sheath when the expandable portion of the sheath is in the fully unexpanded position or orientation. In another and/or alternative non-limiting embodiment, the stretchable outer polymer layer is formed of one or more polymer layers. In another and/or alternative non-limiting embodiment, the stretchable outer polymer layer is optionally formed of polytetrafluoroethylene (PTFE); however, other polymers can be used.

In another and/or alternative non-limiting aspect of the present disclosure, one or more of the polymer portions of the expandable sheath can optionally be partially or fully formed of a shape memory polymer (SMP). Non-limiting shape memory polymers that can be used include a) polyacrylate-based SMPs (e.g., t-butylacrylate-co-poly(ethyleneglycol) dimethacrylate (tBA-co-PEGDMA) polymers, etc.), b) (meth)acrylate-based SMPs, c) polyurethane-based SMPs, d) blends of polyurethane and polyvinylchloride-based SMPs, e) polylactide-based SMPs, f) poly(ε-caprolactone)-based SMPs, ee) poly(vinyl alcohol)-based SMPs, g) ethylene-vinyl acetate-based SMPs, h) polyetheretherketone-based SMPs, i) polynorbornene-based SMPs, j) block copolymer of polyethylene terephthalate (PET) and polyethyleneoxide (PEO) based SMPS, k) block copolymers containing polystyrene and poly(1,4-butadiene) based SMPS, and/or 1) ABA triblock copolymer made from poly(2-methyl-2-oxazoline) and polytetrahydrofuran based SMPs.

In another and/or alternative non-limiting aspect of the present disclosure, the expandable portion of the expandable sheath can optionally include one or more internal structures (e.g., frame, support rods, reinforcement structures, anti-kinking structures, etc.) that are partially or fully embedded in one or more of the polymer portions of the expandable sheath, and wherein the one or more internal structures are partially or fully formed of a shape memory material. The shape memory material can be a shape memory alloy (SMA) and/or a shape memory polymer (SMP). Non-limiting shape member materials that can be used to partially or fully form the shape memory material include a) copper-aluminum-nickel alloy, b) nickel-titanium alloy, c) zinc-copper-gold-iron alloy, d) Fe—Mn—Si alloy, e) Cu—Zn—Al alloy, f) Cu—Al—Ni alloy, g) Ag—Cd alloy, h) Au—Cd alloy, i) Co—Ni—Al alloy, j) Co—Ni—Ga alloy, k) Cu—Al—Be—X (X═Zr, B, Cr, Gd) alloy, 1) Cu—Al—Ni—Hf alloy, m) Cu—Sn alloy, n) Cu—Zn alloy, o) Cu—Zn—X (X═Si, Al, Sn) alloy, p) Fe—Pt alloy, q) Mn—Cu alloy, r) Ni—Fe—Ga alloy, s) Ni—Ti—Hf alloy, t) Ni—Ti—Pd alloy, u) Ni—Mn—Ga alloy, v) Ni—Mn—Ga—Cu alloy, w) Ni—Mn—Ga—Co alloy, x) Ti—Nb alloy, y) polyacrylate-based SMPs (e.g., t-butylacrylate-co-poly(ethyleneglycol) dimethacrylate (tBA-co-PEGDMA) polymers, etc.), z) (meth)acrylate-based SMPs, aa) polyurethane-based SMPs, bb) blends of polyurethane and polyvinylchloride-based SMPs, cc) polylactide-based SMPs, dd) poly(s-caprolactone)-based SMPs, ee) poly(vinyl alcohol)-based SMPs, ff) ethylene-vinyl acetate-based SMPs, gg) polyetheretherketone-based SMPs, hh) polynorbornene-based SMPs, ii) block copolymer of polyethylene terephthalate (PET) and polyethyleneoxide (PEO) based SMPS, jj) block copolymers containing polystyrene and poly(1,4-butadiene) based SMPS, and/or kk) ABA triblock copolymer made from poly(2-methyl-2-oxazoline) and polytetrahydrofuran based SMPs. As defined herein, a flexible, foldable or elastomeric polymer that is merely flexible or foldable, but does not partially or fully return to its original shape after a force is removed from the polymer, is not a shape memory material. In one non-limiting embodiment, the one or more polymer portions used to form a portion or all of the expandable portion of the expandable sheath are not shape memory materials.

In another and/or alternative non-limiting aspect of the present disclosure, the expandable sheath can optionally include a hemostasis valve at or near one end of the expandable sheath.

In another and/or alternative non-limiting aspect of the present disclosure, the outer surface of the expandable portion of the expandable sheath and/or the inner surface of the lumen or cavity of the expandable portion of the expandable sheath is optionally coated with a material (e.g., polymer, etc.) that reduces the surface friction and/or increases the lubricity of the outer surface of the expandable portion of the expandable sheath and/or the inner surface of the lumen or cavity so as to a) facilitate in the insertion of the expandable portion of the expandable sheath into a body passageway and/or b) facilitate in the movement of a medical device through the lumen or cavity of the expandable portion of the expandable sheath. In one non-limiting embodiment, the inner surface of the inner layer portion of the expandable sheath can optionally be or include a lubrication coating and/or liner to facilitate in the movement of a medical device through the expandable sheath. In another and/or alternative non-limiting embodiment, the inner surface of the inner layer portion has a static coefficient of friction of no more about 0.2 (e.g., 0.0001-0.2 and all values and ranges therebetween), and typically no more than 0.1. Non-limiting examples of lubrication coating materials include PTFE, polyethylene, polyvinylidene fluoride, and combinations thereof. In one non-limiting configuration, the inner surface of the inner layer portion includes a coating of PTFE. In another and/or alternative non-limiting embodiment, the outer surface of the outer layer portion of the expandable sheath can optionally be or include a lubrication coating and/or liner to facilitate in the movement of the expandable sheath in a body passageway. In another and/or alternative non-limiting embodiment, the outer surface of the outer layer portion has a static coefficient of friction of no more about 0.1 (e.g., 0.0001-0.1 and all values and ranges therebetween). Non-limiting examples of lubrication materials include PTFE, polyethylene, polyvinylidene fluoride, and combinations thereof. In one non-limiting configuration, the outer surface of the outer layer portion includes a coating of PTFE. In another and/or alternative non-limiting embodiment, the outer surface of the outer layer portion of the expandable sheath can optionally include a hydrophilic coating to facilitate in the insertion of the expandable sheath into body passageway. Non-limiting examples of hydrophilic coatings include the Harmony™ Advanced Lubricity Coatings and other Advanced Hydrophilic Coatings available from SurModics, Inc., Eden Prairie, Minn. DSM medical coatings (available from Koninklijke DSM N. V, Heerlen, the Netherlands).

In another and/or alternative non-limiting aspect of the present disclosure, outer surface of the expandable portion of the expandable sheath and/or the inner surface of the lumen or cavity of the expandable portion of the expandable sheath can optionally be coated with one or more drugs, etc.

In another and/or alternative non-limiting aspect of the present disclosure, the expandable sheath can optionally be partially or fully formed by an extrusion process, stamping process, heat set process, and/or casting process. In one non-limiting embodiment, the expandable sheath is at least partially formed by an extrusion process.

In another and/or alternative non-limiting aspect of the present disclosure, one or both ends of the expandable sheath are optionally subjected to a connection process to temporarily connect together two or more polymer portions or regions of the expandable sheath. One non-limiting connection process is a reflow process to secure one polymer portion to another polymer portion by a heat bonding process (e.g., heat fusion process or heat melt process). As can be appreciated, additional or alternative connection processes can be used (e.g., adhesive, mechanical connection, crimping, etc.). When two or more polymer portions are assembled together to be connected together, and an optional frame can be used in the expandable sheath, and wherein the frame can optionally be partially or fully positioned between two polymer portions. When two or more polymer portions are secured together, a connection process (e.g., reflowed, adhesive, melted seam, pressure connection, etc.) optionally can be used to connected together the two or more polymer portions at one or both end regions of the expandable sheath. When one or more portions of the expandable sheath are temporarily connected together, the temporary connection can be formed by various arrangements (e.g., adhesive, melt connections, snap connection, hook and loop fastener connections, clamp connection, etc.). In use, the connected portions of the one or both ends of the expandable sheath can be optionally opened or separated prior to the insertion of the medical device through the expandable sheath. The temporary connection of one or more both ends of the expandable sheath can be used to facilitate in the initial insertion of a portion of the expandable sheath through an incision and into a body passageway, and thereafter opened prior to the insertion of the medical device through the expandable sheath. An expansion tool can optionally be inserted into the expandable sheath to break open the temporarily connected portions of the expandable sheath.

In another and/or alternative non-limiting aspect of the present disclosure, the expandable sheath is optionally processed to connect together two or more polymer portions along 51-100% (and all values and ranges therebetween) of the longitudinal length of the expandable sheath using one or more connection processes. In another and/or alternative non-limiting embodiment, the connection process optionally includes a heat or reflow process, and/or adhesive.

In another and/or alternative non-limiting aspect of the present disclosure, the expandable sheath optionally includes one or more radiopaque marker or fillers. The radiopaque filler or marker (when used) can be located on the outer surface of the expandable sheath, embedded on one or more layers of the expandable sheath, and/or be located between on or more layers of the expandable sheath. Non-limiting materials that can be used as a radiopaque filler or marker include barium sulfite, bismuth trioxide, titanium dioxide, and/or bismuth subcarbonate.

In another and/or alternative non-limiting aspect of the present disclosure, the inner diameter of the cavity through the longitudinal length of the expandable sheath prior to the expandable sheath being expanded is optionally 0.08-0.2 inches (and all values and ranges therebetween), and the maximum inner diameter of the cavity through the longitudinal length of the expandable sheath after the expandable sheath is expanded is optionally at least 0.15-0.3 inches (and all values and ranges therebetween).

In another and/or alternative non-limiting aspect of the present disclosure, the total wall thickness of the expandable sheath is optionally less than 0.8 mm, and typically less than 0.5 mm.

In another and/or alternative non-limiting aspect of the present disclosure, the longitudinal length of the expandable sheath is optionally at least 2 inches and typically 2-40 inches (and all values and ranges therebetween).

In another and/or alternative non-limiting aspect of the present disclosure, the shape of the expandable sheath can optionally be tubular-shaped or a cylindrical tube. The shape and size of the expandable sheath can be uniform along a majority (e.g., 60-99.99% and all values and ranges therebetween) or the full longitudinal length of the expandable sheath. In one non-limiting embodiment, the expandable portion of the expandable sheath optionally has a generally circular cross-sectional outer profile and/or inner cavity profile when expandable portion of the expandable sheath is in the fully expanded position or orientation.

In another and/or alternative non-limiting aspect of the present disclosure, the expandable sheath in accordance with the present disclosure can be used a) to minimize trauma to a body passageway (e.g., blood vessel, etc.) by allowing for temporary expansion of a portion of the expandable sheath to accommodate a medical device and/or a delivery system for a medical device, and thereafter the expandable sheath is configured to return to its original diameter or close to its original diameter once the medical device and/or a portion of the delivery system passes through sheath, b) to reduce the length of time a procedure takes, c) to reduce the risk of a longitudinal or radial body passageway tear, d) to reduce risk of plaque dislodgement in a body passageway, e) to reduce or eliminate the need for multiple insertions sheaths or other devices for the dilation of a body passageway, f) for many types of minimally invasive surgery, such as any surgery requiring introduction of a medical device (e.g., stent, prosthetic heart valve, grafts, etc.) into a body passageway (e.g., veins, arteries, esophagus, ducts of the biliary tree, intestine, urethra, fallopian tube, other endocrine or exocrine ducts, etc.).

In another and/or alternative non-limiting aspect of the present disclosure, the expandable sheath in accordance with the present disclosure optionally includes an optional conical-shaped front portion on the proximal region of the expandable sheath. The optional conical-shaped front portion is configured to taper in size from the front end of the expandable sheath to the generally tubular-shaped portion of the expandable sheath. The conical-shaped front portion optionally includes an opening therethrough such that a medical device can be inserted into the conical-shaped front portion and thereafter into the cavity of the generally tubular-shaped portion of the expandable sheath. In another and/or alternative non-limiting embodiment, when the expandable sheath optionally includes a frame, 0-50% (and all values and ranges therebetween) of the longitudinal length of the conical-shaped front portion is absent the frame of the expandable sheath. In another and/or alternative non-limiting embodiment, the maximum cross-sectional area of the cavity of the conical-shaped front portion is optionally generally at least 1.5 times (e.g., 1.5-10 times and all values and ranges therebetween) the cross-sectional area of the internal passageway or cavity of the tubular portion of the expandable sheath when in the collapsed or unexpanded position. In another and/or alternative non-limiting embodiment, the conical-shaped front portion can be formed of the same or different material as the one or more polymer portions used to form the expandable sheath. The conical-shaped front portion can be a separate component that is connected to the front end or portion of the body of the expandable sheath by various arrangements (e.g., melt connection, adhesive, etc.).

In another and/or alternative non-limiting aspect of the present disclosure, the expandable sheath includes an optional lip that overlaps a portion of the outer surface of the body (e.g., tubular body, etc.) of the expandable sheath when the expandable sheath is in the collapsed or unexpanded position. All or a portion of the lip can optionally have a thickness that is less than a thickness of the outer layer of the expandable sheath. The lip, when use, can be formed of the same or different material from the outer layer of the expandable sheath. The lip, when used, has a length that is 0.01-50% (and all values and ranges there therebetween) the length of the outer perimeter of the tubular body of the expandable sheath. In one non-limiting embodiment, the lip overlaps a portion of the outer surface of the body of the expandable sheath when the expandable sheath is in the collapsed or unexpanded position. In another and/or alternative non-limiting embodiment, the lip does not overlaps a portion of the outer surface of the body of the expandable sheath when the expandable sheath is in the fully expanded position or orientation.

In another and/or alternative non-limiting aspect of the present disclosure, the expandable sheath includes one or more breakable connection points or regions on a) one or more polymer portions of the expandable sheath along the longitudinal length of the expandable sheath, b) the optional conical shaped front portion, and/or c) the spiral distal end region of the expandable sheath to maintain the expandable sheath in the collapsed or unexpanded position. The one or more breakable connection points or regions can be used to maintain one or more portions of the expandable sheath to maintain the expandable sheath in the collapsed or unexpanded position until at least a distal region of the expandable sheath is inserted into the body passageway so as to facilitate the insertion of at least a portion of the expandable sheath into the body passageway. After at least a portion of the expandable sheath that includes the one or more breakable connection points or regions is inserted into the body passageway, one or more of the breakable connection points or regions can be broken open by a) a medical device being inserted through the internal passageway or cavity of the body of the expandable sheath to cause a at least a portion of the expandable sheath to move to the non-collapsed or expanded position, which expansion results in one or more or all of the breakable connection points or regions to break open, or b) an expansion instrument being inserted through the internal passageway or cavity of the body of the expandable sheath to cause at least a portion of the expandable sheath to move to the non-collapsed or expanded position, which expansion results in one or more or all of the breakable connection points or regions to break open. In one non-limiting embodiment, one or more or all of the breakable connection points or regions are broken open after the portion of the expandable sheath that includes the one or more or all of the breakable connection points or regions is inserted into the body passageway. The one or more breakable connection points or regions can be formed of a) an adhesive, b) one or more melted portions that optionally have a thickness that is less than a thickness of the wall of the expandable sheath (e.g., 1-25% a thickness of the wall of the expandable sheath and all values and ranges therebetween), c) breakable stitching, d) hook and look fastener, and/or e) mechanical and/or friction connection. When a plurality of breakable connection points are located on the expandable sheath, the plurality of breakable connection points are generally spaced from one another. As can be appreciated, one or more of the breakable connection points or regions can extend 1-100% (and all values and ranges therebetween) of the longitudinal length of the expandable sheath. The one or more breakable connection points can be formed by one or more arrangements (e.g., adhesive, melted connection, hook and look fastener, etc.).

In another and/or alternative non-limiting aspect of the present disclosure, the expandable sheath can optionally include a specially cut tip region at the distal end or region of the expandable sheath to facilitate in the operation of the expandable sheath. In one non-limiting embodiment, a spiral-type cut is formed in the distal most tip region of an expandable sheath. Such spiral cut can be used to overcome and/or address the issues of a) the catheter being deflected away from being coaxial with the longitudinal axis of the expandable sheath as the catheter is move out from the distal end of the expandable sheath, and/or b) the tip region of the expandable sheath being caused to move away from being coaxial with the longitudinal axis of the expandable sheath as the catheter moves out from the distal end of the sheath. In one non-limiting embodiment the distal end region of the expandable sheath is absent a longitudinal cut in the sheath tip that is completely parallel to the central axis of the lumen or cavity of the expandable sheath. The spiral cut in the distal end region of the sheath can be used to facilitate in ensuring that there is always a portion of the sheath tip that wraps around the catheter as the catheter moves through the distal end region of the expandable sheath, thereby keeping the catheter in a coaxial configuration relative central axis of the lumen of the expandable sheath at the location the catheter exits the distal end of the expandable sheath. In one non-limiting specific configuration, the spiral cut in the distal end region of the sheath is cut at an angle of at least 5°and less than 180° (and all values and ranges therebetween) from the axis that is parallel to the central lumen of the expandable sheath. The spiral cut can extend along the longitudinal length of the expandable sheath a distance of a) 0.1-25% of the longitudinal length of the expandable sheath, and/or b) at least 0.4 mm (e.g., 0.4-20 mm and all values and ranges therebetween) of the longitudinal length of the expandable sheath.

In another and/or alternative non-limiting aspect of the present disclosure, the tip of the sheath is optionally a separate component that is connected (e.g., adhesive connection, melted connection, friction connection, weld or soldered connection, etc.) to the distal end or distal end region of the main body of the expandable sheath. When the tip of the sheath is a separate component, and the tip optionally includes a spiral cut, the spiral cut can be exclusively formed in such tip, and optionally be absent from the main body of the expandable sheath; however, this is not required. When the tip is a separate component from the main body of the expandable sheath, the tip can optionally form 50-100% (and all values and ranges therebetween) of the distal end region of the expandable sheath. When the tip is a separate component from the main body of the expandable sheath, the tip can optionally be formed of the same material or different material from the main body of the expandable sheath. In one non-limiting configuration, the tip includes or is formed of thermoplastics (Pebax, Nylon, etc.) or metals (nitinol, stainless steel, etc.). In another and/or alternative non-limiting configuration, the tip is formed of a different material from the main body of the expandable sheath. In another and/or alternative non-limiting configuration, the tip is formed of a polymer material.

In another and/or alternative non-limiting aspect of the present disclosure, the expandable sheath in accordance with the present disclosure can be formed by different processing steps, and wherein one non-limiting set of processing steps involves the formation of the expandable sheath by an expandable portion of the expandable sheath that is at least partially formed of a continuous first polymer portion that has first and second thicknesses, and wherein the processing steps include:

Forming the first polymer portion of the expandable portion of the expandable sheath using a first polymer (e.g., polyether-block-amide (PEBA) (e.g., Pebax™, etc.), polyvinyl acetate or polyvinyl acetate or poly(ethenyl ethanoate) (PVA or PVAC), etc.). In one non-limiting embodiment, the first polymer is optionally extruded through a die that forms a shaped expandable portion (e.g., tubular shape, etc.) of the expandable sheath in a shape that represents the expandable portion of the expandable sheath in the fully expanded position or orientation, and wherein the first polymer portion includes a portion that has a first thick layer portion (e.g., 0.002-0.03 inches and all values and ranges therebetween) and a portion that has a second thin layer portion (e.g., 0.001-0.01 inches and all values and ranges therebetween), and wherein the average or maximum thickness of the thin layer portion is less than the average or minimum thickness of the thick layer portion. Generally, the lumen or cavity or passageway through the expandable portion of the expandable sheath that extends along the central longitudinal axis of the expandable portion of the expandable sheath can optionally be generally circular or oval (however, other shapes can be used). In one non-limiting configuration, the average diameter of the lumen when the expandable portion of the expandable sheath is in the fully expanded position or orientation is at least 4 mm (e.g., 4-15 mm and all values and ranges therebetween). The first polymer portion can be formed by one or more processes (e.g., extrusion, molding, stamping, 3D printing, and/or cutting). A taper may optionally exist in the transition region between the first and second thickness of the first polymer portion.

Optionally positioning and optionally connecting an optional liner (e.g., PTFE liner, etc.) in the interior of the lumen of the expandable portion of the expandable sheath. When the optional liner is formed of a polymer material, the optionally liner can also be referred to as a second polymer portion of the expandable sheath. When the optional liner is used, the optional liner can be positioned in and/or connected to the first polymer portion by a) extruding the first polymer portion about the optional liner, or b) inserting the optional liner into the lumen of the expandable portion of the expandable sheath after the extrusion step of the first polymer portion, and thereafter optionally connecting the liner to the inner surface of the first polymer portion by use of one or more connections processes (e.g., adhesive, melted connection, etc.). The optional liner can optionally be a tubular shaped liner that optionally has a wall thickness of at least 0.001 inches (e.g., 0.001-0.01 inches and all values and ranges therebetween) and optionally has a circular or oval cross-sectional shape in the expanded orientation. Generally, the shape of the tubular liner is the same as or similar to the shape of the lumen when the first polymer portion is in the fully expanded orientation, and has a cross-sectional size that is generally ±10% (and all values and ranges therebetween) of the cross-sectional size of the lumen when the first polymer portion is in the fully expanded orientation. The outer surface of the optional liner can optionally include surface etching and/or roughening to facilitate in the bonding/connecting the optional liner to the first polymer portion. The optional liner generally has a longitudinal length of 50-100+% (and all values and ranges therebetween) the longitudinal length of the lumen formed by the first polymer portion. The bonding/connecting of the optional liner to the first polymer portion can be by a heat process (e.g., heat flow process, thermocompression process, etc.) and/or by use of an adhesive, or by some other arrangement. When the optional liner has a melting point that is greater than the melting point of the first polymer portion, the optional liner can optionally include an etched and/or roughened outer surface. When the optional liner has a melting point that is the same or less than the melting point of the first polymer portion, the optional liner can include or be absent an etched and/or roughened outer surface. In one non-limiting configuration, the first polymer portion is subjected to a heat flow process or thermocompression process to connect the optional liner, when used, to the first polymer portion. During the heat flow process or thermocompression process, a mandrel can be optionally inserted in the lumen of the optional liner to a) maintain the shape of the first polymer portion and the shape of the optional liner during the heat flow process or thermocompression process, b) facilitate in providing heat to the first polymer portion and the optional liner during the heat flow process or thermocompression process, and/or c) facilitate in providing a desired heat distribution to the first polymer portion and the optional liner during the heat flow process or thermocompression process.

An optional heat shrink layer can be applied to the outer surface of the first polymer portion prior to the expandable portion of the expandable sheath being subjected to the heat flow process or thermocompression process. The optional heat shrink layer can be configured to shrink during the heat flow process or thermocompression process and apply an outer compression force to the outer surface of the first polymer portion to facilitate in the connecting of the optional liner to the first polymer portion. Generally, the optional heat shrink layer is formed of a different material from the first polymer portion and the optional liner. Generally, the optional heat shrink layer is a stretchable layer; however, this is not required. In one non-limiting configuration, the optional heat shrink layer is formed of fluorinated ethylene propylene (FEP), polyolefin, etc. In one non-limiting configuration, the thickness of the heat shrink layer is at least 0.001 inches (e.g., 0.001-0.01 inches and all values and ranges therebetween). In another and/or alternative non-limiting configuration, the melting point of the optional heat shrink layer is greater than the melting point of the first polymer portion so as to inhibit or prevent bonding of the optional heat shrink layer to the outer surface of the first polymer portion during the heat flow process or thermocompression process. When a mandrel is used, the mandrel facilitates in maintaining the shape of the first polymer portion and the optional liner in the fully expanded position or orientation as the heat shrink layer applies the outer compression force to the outer surface of the first polymer portion. After the connection of the optional liner to the first polymer portion is completed, the mandrel, when used, and the heat shrink layer, when used, are removed from the first polymer portion and/or the optional liner.

Folding first polymer portion into the fully unexpanded position or orientation. The first polymer portion, and the optional liner can be folded about a smaller cross-sectional mandrel (e.g., tapered mandrel, etc.) to form the expandable portion of the expandable sheath in the fully unexpanded position or orientation. In the fully unexpanded position or orientation, the end portions of the first thick layer portion of the first polymer portion partially or fully overlap one another, and a portion or all of the second thin layer portion of the first polymer portion (e.g., 60-100% of the longitudinal length of the second thin layer portion as measured between the two ends of the first thick layer portion and all values and ranges therebetween) is positioned between the overlapped end portions of the first thick layer portion of the first polymer portion, and optionally 0-50% (and all values and ranges therebetween) of the second thin layer portion is located in the lumen of the expandable portion of the expandable sheath when in the fully unexpanded position or orientation. In the fully unexpanded position or orientation, the average diameter of the lumen of the expandable portion of the expandable sheath is at least 10% less (e.g., 10-400% less and all values and ranges therebetween) than the average diameter of the lumen of the expandable portion of the expandable sheath in the fully expanded position or orientation. The longitudinal length of the second thin layer portion of the first polymer portion can be selected to limit the amount of overlap of the two end portions of the first thick payer portion of the first polymer portion when the expandable portion of the expandable sheath in the fully unexpanded position or orientation. In another and/or alternative non-limiting configuration, no more than 30% (e.g., 0-30% and all values and ranges therebetween) of the second thin layer portion of the first polymer portion, that is not located between overlay of the two end portions of the first thick layer portion of the first polymer portion, is located in the lumen of the expandable portion of the expandable sheath when the expandable portion of the expandable sheath is in the fully unexpanded position or orientation. When the expandable portion of the expandable sheath includes an optional liner, a portion of the optional liner is optionally caused to be positioned between the overlapped end portion of the first thick layer portion of the first polymer portion when the end portions of the first thick layer portion of the first polymer portion partially or fully overlap one another, and the optional liner is folded about the optional smaller cross-sectional mandrel (e.g., tapered mandrel, etc.).

Applying an optional non-stick temporary layer (e.g., PTFE polymer layer, etc.). The optional non-stick temporary layer can be applied between one or both sides of the second thin layer portion of the first polymer portion that is positioned between the overlapping end portions of the first thick layer portion of the first polymer portion so as to inhibit or prevent sticking of the second thin layer portion of the first polymer portion to the overlapping end portions of the first thick layer portion of the first polymer portion during the annealing of the expandable portion of the expandable sheath in the fully unexpanded position or orientation. The optional non-stick temporary layer is generally applied to 1-100% (and all values and ranges therebetween) the longitudinal length of the second thin layer portion of the first polymer portion and can be applied to one or both sides of the second thin layer portion of the first polymer portion. When the optional non-stick temporary layer is applied to both sides of the second thin layer portion of the first polymer portion, the length of the optional non-stick temporary layer on each side of the second thin layer portion of the first polymer portion can be the same or different. Generally, the melting point of the optional non-stick temporary layer is greater than the melting point of the first polymer portion.

Annealing of the expandable portion of the expandable sheath while the expandable portion of the expandable sheath is in the fully unexpanded position or orientation. Generally, the first polymer portion, and the optional liner, and the optional non-stick temporary layer are folded about or positioned about the smaller cross-sectional mandrel (e.g., tapered mandrel, etc.) during the annealing process. Once the first polymer portion, and the optional liner and the optional non-stick layer are folded or positioned about the smaller cross-sectional mandrel (e.g., tapered mandrel, etc.), the first polymer portion is heated to anneal the first polymer portion so that the first polymer portion obtains a relaxed state (e.g., zero stress state) while in the fully unexpanded position or orientation. The temperature to which the first polymer portion is heated is less than the melting point of the first polymer portion (e.g., 5-60 °C less than the melting point and all values and ranges therebetween). As such, during the annealing process, the first polymer portion is softened, but is not reflowed or melted. The annealing process is typically at least 5 seconds (e.g., 5 seconds to 2 hours and all values and ranges therebetween). An optional heat shrink layer can be applied to the outer surface of the first polymer portion prior to the expandable portion of the expandable sheath being set or annealed in the fully unexpanded position or orientation. The optional heat shrink layer is configured to shrink during the annealing process and apply an outer compression force to the outer surface of the first polymer portion to facilitate maintaining the expandable portion of the expandable sheath in the fully unexpanded position or orientation during the annealing process. The smaller cross-sectional mandrel (e.g., tapered mandrel, etc.) prevents further collapsing of the first polymer portion as the heat shrink layer during the annealing process applies an outer compression force to the outer surface of the first polymer portion. Generally, the optional heat shrink layer is formed of a different material from the first polymer portion and the optional liner. In one non-limiting configuration, the optional heat shrink layer is formed of fluorinated ethylene propylene (FEP), polyolefin, etc.; however, other materials can be used. In another and/or alternative non-limiting configuration, the melting point of the optional heat shrink layer is greater than the melting point of the first polymer portion so as to inhibit or prevent bonding of the optional heat shrink layer to the outer surface of the first polymer portion during the annealing process. When the smaller cross-sectional mandrel (e.g., tapered mandrel, etc.) is used during the annealing process, the smaller cross-sectional mandrel (e.g., tapered mandrel, etc.) facilitates in preventing over collapsing of the shape of the first polymer portion and the optional liner beyond the desired fully unexpanded position or orientation as the heat shrink layer applies the outer compression force to the outer surface of the first polymer portion. After the annealing of the first polymer portion is completed, the first polymer portion is allowed to cool, and thereafter the smaller cross-sectional mandrel (e.g., tapered mandrel, etc.), the optional heat shrink layer, and the optional non-stick layer, when used, are removed from the first polymer portion. In one non-limiting configuration, the first polymer portion is optionally allowed to cool to ambient temperature (e.g., 60°F-85 °F and all values and ranges therebetween) prior to the removal of the smaller cross-sectional mandrel (e.g., tapered mandrel, etc.), the optional heat shrink layer, when used, and the optional non-stick layer, when used, from the first polymer portion.

Optionally forming a taper end tip to the expandable portion of the expandable sheath. When a tapered end portion of the expandable portion of the expandable sheath is to be formed, such tapered tip can be optionally formed by applying a heat shrink layer over the tip end portion of the expandable portion of the expandable sheath. In one non-limiting configuration, the tip end portion is of the expandable portion of the expandable sheath is generally at least 0.1 mm in length (e.g., 0.1-10 mm and all values and ranges therebetween). A mandrel can optionally be positioned in the lumen located at the tip portion of the expandable portion of the expandable sheath. The mandrel, when used, is used to maintain the inner diameter of the lumen at the tip portion of the expandable portion of the expandable sheath at a desired diameter or cross-sectional area (e.g., a diameter or cross-sectional area that is equal to the size of the dilator that will be used inside the expandable portion of the expandable sheath [e.g. 6-15 Fr and all values and ranges therebetween; 2-5 mm and all values and ranges therebetween], etc.), and/or desired cross-sectional shape. Heat is then applied to the heat shrink layer to form down or compress the tip portion inner diameter to the outer diameter of the mandrel located in the lumen at the tip portion of the expandable portion of the expandable sheath, and thereby forming a taper on the leading edge or distal region of the expandable portion of the expandable sheath. In one non-limiting embodiment, the heat applied to the heat shrink layer can be sufficient to cause the first polymer portion to a) be annealed, or b) be reflowed or melted to form i) a bond between one or more polymer portions or layers, b) form a temporary breakable bond between the ends of the first thick layer portion at the tip portion, and/or c) bond a tip portion to a distal end portion of the expandable sheath; however, this is not required. After the heating of the tip portion of the expandable portion of the expandable sheath is completed and the tapered tip portion is formed, the heat shrink layer is removed from the expandable portion of the expandable sheath. As can be appreciated, the tapering of the leading edge of the expandable portion of the expandable sheath can be accomplished without the use of a heat shrink layer and/or a mandrel. For example, the tapered tip portion can alternatively be formed using a heated die which forms the tip portion in its tapered shape without use of heat shrink layer. When the tip portion is heated to a reflow or melting temperature, the leading edge of the tip portion may form a continuous or non-continuous reflow ring around the circumference of the tip portion. The longitudinal length of this continuous or non-continuous reflow ring should be controlled and minimized so that acceptable opening forces of the expandable portion of the expandable sheath at the tapered tip portion of the expandable portion of the expandable sheath can be achieved so that the expandable portion of the expandable sheath will properly move from the unexpanded to the expanded position or orientation as a medical device passes through the top portion of the expandable portion of the expandable sheath without damaging the medical device as it passes through the top portion of the expandable portion of the expandable sheath. As can be appreciated, an expansion tool can be inserted into lumen of the expandable sheath after the expandable sheath is inserted into a patient to cause the partial or full opening of the reflowed tip portion prior to the insertion of a medical device through the expandable sheath; however, this is not required. As can be appreciated, a portion of an optional liner can be located in the tip portion; however, this is not required.

Optionally adding an elastomeric continuous outer sleeve to a) at least a proximal portion of the expandable portion of the expandable sheath, b) at least a proximal portion of the expandable portion of the expandable sheath and the distal portion of the hub when the expandable sheath optionally includes a hub portion, c) 50-100% (and all values and ranges therebetween) of the longitudinal length of the expandable portion of the expandable sheath, and/or d) 50-100% (and all values and ranges therebetween) of the longitudinal length of the expandable portion of the expandable sheath and the distal portion of the hub when the expandable sheath optionally includes a hub portion. The proximal end or proximal end portion of the expandable portion of the expandable sheath can be configured to be connected to a connection hub portion of the expandable sheath when the expandable sheath optionally includes a hub portion. Most or all of the connection hub portion, when used, is typically formed of a rigid, non-expandable and non-flexible material; however, this is not required. The distal end of the connection hub portion, when used, includes a distal hub connection opening that is configured to be connected to the proximal end or proximal end portion of the expandable portion of the expandable sheath. The diameter of the distal hub connection opening is generally greater than the inner diameter of the lumen of the expandable portion of the expandable sheath when in the fully unexpanded position or orientation prior to the expandable sheath being connected to the hub. In one non-limiting configuration, the diameter of the distal hub connection opening is optionally the same as or slightly smaller (e.g., 0.0001-15% smaller and all values and ranges therebetween) than the inner diameter of the lumen of the expandable portion of the expandable sheath when in the fully expanded position or orientation. The distal hub connection opening can be connected to the proximal end or proximal end portion of the expandable portion of the expandable sheath prior to or after the annealing of the expandable portion of the expandable sheath. In one non-limiting embodiment, the proximal end or end portion of the expandable portion of the expandable sheath can be connected to the distal hub connection opening by a) heat flowing or melting the proximal end or proximal end portion of the expandable portion of the expandable sheath to distal hub connection opening, b) adhesively connecting the proximal end or proximal end portion of the expandable portion of the expandable sheath to distal hub connection opening, and/or c) mechanically clamping/connecting the proximal end or proximal end portion of the expandable portion of the expandable sheath to distal hub connection opening. In one non-limiting method, the proximal end or proximal end portion of the expandable portion of the expandable sheath is connected to the distal hub connection opening by heat flowing or melting the proximal end or proximal end portion of the expandable portion of the expandable sheath to distal hub connection opening while a mandrel is positioned in the lumen of the proximal end or proximal end portion of the expandable portion of the expandable sheath to maintain the diameter of the lumen at a desired size to form a desired connection between the proximal end or proximal end portion of the expandable portion of the expandable sheath and the distal hub connection opening. When the proximal end or proximal end portion of the expandable portion of the expandable sheath is connected to the distal hub connection opening, the proximal end or proximal end portion of the expandable portion of the expandable sheath can be abutted against the end of the distal hub connection opening during the connection process. When the proximal end portion of the expandable portion of the expandable sheath is connected to the distal hub connection opening, a portion of the proximal end portion of the expandable portion of the expandable sheath can be a) inserted about the outer surface of a portion of the end portion of the distal hub connection opening during the connection process, or b) inserted into the interior of the distal hub connection opening during the connection process, or c) be abutted against the distal hub connection during the connection process. After the connection is formed, the mandrel can be removed from the lumen. The connection between the proximal end or proximal end portion of the expandable portion of the expandable sheath and the distal hub connection opening can be at the same time or a different time than when the optional liner is connected to the first polymer portion. Prior to, during or after the connection between the proximal end or proximal end portion of the expandable portion of the expandable sheath and the distal hub connection opening is formed, an optional elastomeric continuous outer sleeve can be positioned over a portion of the hub portion and/or all or a portion of the proximal portion of the expandable portion of the expandable sheath. The longitudinal length of the optional elastomeric continuous outer sleeve is a) generally at least 0.5 cm (e.g., 0.5-25 cm and all values and ranges therebetween), and/or b) at least 0.1% (e.g., 0.1-60% and all values and ranges therebetween) of the longitudinal length of the expandable sheath. When the optional elastomeric continuous outer sleeve is used, the optional elastomeric continuous outer sleeve is used to a) provide a circumferentially continuous outer surface at the interface between the sheath shaft outer surface and the vessel arteriotomy, to provide an effective hemostasis at such interface, b) provide another layer of leak protection if there is any breach in the expandable thinner wall portion of the shaft during use, c) facilitate in forming a leak-proof connection between the proximal end or proximal end portion of the expandable portion of the expandable sheath and the distal hub connection opening when the expandable sheath optionally includes a hub portion, d) provide strength to the connection between the proximal end or proximal end portion of the expandable portion of the expandable sheath and the distal hub connection opening when the expandable sheath optionally includes a hub portion, e) inhibit or prevent damage to or disconnection of the connection between the proximal end or proximal end portion of the expandable portion of the expandable sheath and the distal hub connection opening when the expandable sheath optionally includes a hub portion, and/or f) facilitate in maintaining the expandable portion of the expandable sheath in the unexpanded orientation. The optional elastomeric continuous outer sleeve, when used, can optionally be reflowed or melted on to the outer surface of the expandable portion of the expandable sheath and the distal portion of the hub portion when the expandable sheath optionally includes a hub portion. During the reflow or melting of the optional elastomeric continuous outer sleeve on the outer surface of the expandable portion of the expandable sheath and the distal portion of the hub portion, an optional heat shrink layer (e.g., FEP or polyolefin) can be applied over a portion or all of the outer surface of the elastomeric continuous outer sleeve to apply a compressive force to the elastomeric continuous outer sleeve during the melting or reflow of the optional elastomeric continuous outer sleeve on the outer surface of the expandable portion of the expandable sheath and the distal portion of the hub portion. Thereafter, the optional heat shrink layer can be removed from the outer surface of the optional elastomeric continuous outer sleeve. The optional elastomeric continuous outer sleeve on the outer surface of the expandable portion of the expandable sheath is considered another polymer portion of the expandable portion of the expandable sheath. The material used to form the optional elastomeric continuous outer sleeve can optionally be a stretchable polymer. The material and/or thickness of the optional elastomeric continuous outer sleeve can be different from one or more of the other polymer portions of the expandable portion of the expandable sheath.

Optionally forming a spiral tip on a distal region of the expandable sheath or connecting a preformed spiral tip to the distal region of the expandable sheath. When the spiral tip is directly formed on the distal region of the expandable sheath, a spiral cut in the distal end region of the expandable is formed. The spiral cut can extend along the longitudinal length of the expandable sheath a distance of a) 0.1-25% of the longitudinal length of the expandable sheath, and/or b) at least 0.4 mm (e.g., 0.4-20 mm and all values and ranges therebetween). When the spiral tip of a separate component, the spiral tip can be connected to the distal region or end of the expandable sheath by various arrangements (e.g., adhesive connection, melted connection, friction connection, weld or soldered connection, etc.).

In another and/or alternative non-limiting aspect of the present disclosure, the expandable sheath in accordance with the present disclosure can be formed by different processing steps, and wherein one non-limiting set of processing steps involves the formation of the expandable sheath by an expandable portion of the expandable sheath that is formed of a first polymer portion that has first thickness and a second polymer portion that has a second thickness and wherein the second polymer portion is connected to the first polymer portion, and wherein the processing steps include:

Forming the first polymer portion of the expandable portion of the expandable sheath using a first polymer (e.g., polyether-block-amide (PEBA) (e.g., Pebax™, etc.), polyvinyl acetate or polyvinyl acetate or poly(ethenyl ethanoate) (PVA or PVAC), etc.). In one non-limiting embodiment, the first polymer is optionally extruded through a die that forms a portion of the shaped expandable portion (e.g., tubular shape, etc.) of the expandable sheath in a shape that represents a portion of the expandable portion of the expandable sheath in the fully expanded position or orientation, and wherein the first polymer portion has a thickness (e.g., 0.0055-0.025 inches and all values and ranges therebetween). As can be appreciated, the first polymer portion of the expandable portion of the sheath can be formed by one or more processes (e.g., extrusion, molding, stamping, 3D printing, and/or cutting). In another non-limiting embodiment, the first polymer portion is optionally extruded through a die that forms the first polymer portion in generally tubular shape or oval shape, and thereafter, a portion of the first polymer portions is cut along 50-100% (and all values and ranges therebetween) of a longitudinal length of the first polymer portion to form a gap between first and second side ends in the outer perimeter of the first polymer portion. As can be appreciated, the first polymer portion can be optionally extruded to include the gap in the first polymer portion. The width of the cut portion is about 2-45% (and all values and ranges therebetween) of the outer perimeter of the expandable portion of the sheath or the first polymer portion prior to being cut The cut portion can have a uniform cut width along 50-100% (and all values and ranges therebetween) of a longitudinal length of the first polymer portion. The end portion on one or both sides of the cut portion can optionally be tapered and/or rounded; however, this is not required.

Forming the second polymer portion of the expandable portion of the expandable sheath using a second polymer (e.g., polyether-block-amide (PEBA) (e.g., Pebax™, etc.), polyvinyl acetate or polyvinyl acetate or poly(ethenyl ethanoate) (PVA or PVAC), etc.). As can be appreciated, the second polymer portion of the expandable portion of the sheath can be formed by one or more processes (e.g., extrusion, molding, stamping, 3D printing, and/or cutting). The second polymer can be formed of the same of different material from the first polymer of the first polymer portion. In one non-limiting embodiment, the second polymer portion is optionally extruded through a die that forms the second polymer portion in the desired shape of the second polymer portion in the expanded orientation (e.g., semi-circular cross-sectional, etc.), and wherein the second polymer portion has a thickness (e.g., 0.001-0.005 inches and all values and ranges therebetween), and wherein the first and second ends of the second polymer portion may or may not be tapered. In one non-limiting embodiment, the average or maximum thickness of the second polymer portion is less than the average or minimum thickness of the first polymer portion. In another non-limiting embodiment, the second the second polymer portion is optionally extruded through a die that forms the second polymer portion in generally tubular shape or oval shape, and thereafter, a portion of the second polymer portions is cut along 50-100% (and all values and ranges therebetween) of a longitudinal length of the second polymer portion to form a gap between first and second side ends in the outer perimeter of the second polymer portion. The width of the cut portion is about 2-45% (and all values and ranges therebetween) of the outer perimeter of the tube. The cut portion can have a uniform cut width along 50-100% (and all values and ranges therebetween) of a longitudinal length of the tube. The end portion on one or both sides of the cut portion can optionally be tapered and/or rounded; however, this is not required.

Connecting one end of the first polymer portion to one end of the second polymer portion, and connecting the other end of the first polymer portion to the other end of the second polymer portion. The connection mechanism to connect the first and second polymer portions together is non-limiting (e.g., heat bonding, adhesive, mechanical connection [e.g., snap connection, clip connection, staple connection, pin connection, stitching, friction fit connection, etc.), melt bonding, welded bond, use of bonding layer or bonding strips, co-extrusion, etc.). As can be appreciated, two or more bonding arrangements can be used (e.g., adhesive and heat bonding, heat bonding and use of bonding layer such as an inner and/or outer polymer coating, etc.). The connecting together of the first and second polymer portions forms a lumen or cavity or passageway through the expandable portion of the expandable sheath that extends along the central longitudinal axis of the expandable portion of the expandable sheath. The lumen, cavity, or passageway can optionally be generally circular or oval (however, other shapes can be used).

The one or more steps of the following process steps or other process steps as discussed above can be optionally used to form the expandable sheath: a) use and/or connection of an optional liner in the interior of the lumen of the expandable portion of the expandable sheath, b) use of an optional heat shrink layer to facilitate in the connecting of the optional liner to the polymer portion, c) folding one or more polymer portion into fully unexpanded position or orientation, d) applying an optional non-stick layer one or both sides of one or more polymer portions that are positioned between the overlapping ends of a portion of the expandable sheath so as to inhibit or prevent sticking, e) annealing of the expandable portion of the expandable sheath while the expandable portion of the expandable sheath is in the fully unexpanded position or orientation, f) use of optional heat shrink layer during annealing, g) using of an optional mandrel during the annealing process, h) optionally forming a taper end tip to the expandable portion of the expandable sheath, i) optionally adding an elastomeric continuous outer sleeve to A) at least a proximal portion of the expandable portion of the expandable sheath, B) at least a proximal portion of the expandable portion of the expandable sheath and the distal portion of the hub when the expandable sheath optionally includes a hub portion, C) 50-100% (and all values and ranges therebetween) of the longitudinal length of the expandable portion of the expandable sheath, and/or D) 50-100% (and all values and ranges therebetween) of the longitudinal length of the expandable portion of the expandable sheath and the distal portion of the hub when the expandable sheath optionally includes a hub portion, and/or j) optionally forming a spiral tip on a distal region of the expandable sheath or connecting a preformed spiral tip to the distal region of the expandable sheath.

In another and/or alternative non-limiting aspect of the present disclosure, the expandable sheath in accordance with the present disclosure can be formed by different processing steps, and wherein one non-limiting set of processing steps involves the formation of the expandable sheath by an expandable portion of the expandable sheath that is formed of a) a second polymer portion that forms an outer layer of the expandable portion of the expandable sheath, and the second polymer portion forms a continuous outer layer, and wherein the second polymer portion is optionally a non-stretchable material, and wherein the second polymer portion optionally has a constant thicknesses, and b) a first polymer portion is a non-continuous layer that has two ends that are not connected together along 50-100% (and all values and ranges therebetween) of the outer circumference of the expandable portion of the sheath when in the fully open or expanded orientation, and wherein the two ends are optionally separated from one another when the expandable portion of the sheath is in the fully expanded orientation, and wherein the first polymer portion is formed of a same or different material from the second polymer portion, and wherein a durometer and/or thickness of the first polymer portion is optionally greater than a durometer and/or thickness of the second polymer portion, and wherein the processing steps include:

Forming the first polymer portion of the expandable portion of the expandable sheath using a first polymer (e.g., polyether-block-amide (PEBA) (e.g., Pebax™, etc.), polyvinyl acetate or polyvinyl acetate or poly(ethenyl ethanoate) (PVA or PVAC), etc.). In one non-limiting embodiment, the first polymer is optionally extruded through a die that forms a portion of the shaped expandable portion (e.g., tubular shape, etc.) of the expandable sheath in a shape that represents a portion of the expandable portion of the expandable sheath in the fully expanded position or orientation, and wherein the first polymer portion has a thickness (e.g., 0.0055-0.025 inches and all values and ranges therebetween). As can be appreciated, the first polymer portion of the expandable portion of the sheath can be formed by one or more processes (e.g., extrusion, molding, stamping, 3D printing, and/or cutting). In another non-limiting embodiment, the first polymer portion is optionally extruded through a die that forms the first polymer portion in generally tubular shape or oval shape, and thereafter, a portion of the first polymer portions is cut along 50-100% (and all values and ranges therebetween) of a longitudinal length of the first polymer portion to form a gap between first and second side ends in the outer perimeter of the first polymer portion. As can be appreciated, the first polymer portion can be optionally extruded to include the gap in the first polymer portion. The width of the cut portion is about 2-45% (and all values and ranges therebetween) of the outer perimeter of the expandable portion of the sheath or the first polymer portion prior to being cut The cut portion can have a uniform cut width along 50-100% (and all values and ranges therebetween) of a longitudinal length of the first polymer portion. The end portion on one or both sides of the cut portion can optionally be tapered and/or rounded; however, this is not required.

Forming the second polymer portion of the expandable portion of the expandable sheath using a second polymer (e.g., polyether-block-amide (PEBA) (e.g., Pebax™, etc.), polyvinyl acetate or polyvinyl acetate or poly(ethenyl ethanoate) (PVA or PVAC), etc.). As can be appreciated, the second polymer portion of the expandable portion of the sheath can be formed by one or more processes (e.g., extrusion, molding, stamping, 3D printing, and/or cutting). The second polymer can be formed of the same of different material from the first polymer of the first polymer portion. In one non-limiting embodiment, the second polymer portion is optionally extruded through a die that forms the second polymer portion in the desired shape of the second polymer portion in the expanded orientation (e.g., circular cross-sectional, oval cross-sectional shape, etc.), and wherein the second polymer portion has a thickness (e.g., 0.001-0.005 inches and all values and ranges therebetween). The second polymer portion is optionally a continuous tubular portion; however, this is not required. In one non-limiting embodiment, the average or maximum thickness of the second polymer portion is less than the average or minimum thickness of the first polymer portion.

Positioning the second polymer portion about the outer surface of the first polymer portion and optionally connecting the second polymer portion to the first polymer portion. The second polymer portion can be connected to the first polymer portion by a) heat flowing, melting, or using a thermocompression process to connect the second polymer portion to the first polymer portion, and/or b) adhesively connecting the second polymer portion to the first polymer portion, and/or c) mechanically clamping/connecting the second polymer portion to the first polymer portion. As can be appreciated, other or additional arrangements can be used to connect the second polymer portion to the first polymer portion. During the connecting process, a mandrel can be optionally inserted in the lumen of the first polymer portion to a) maintain the shape of the first and second polymer portions during the connection process, b) facilitate in providing heat to the first and/or second polymer portions during the connection process that uses heat, and/or c) facilitate in providing a desired heat distribution to the first and/or second polymer portions during the connection process that uses heat. After the completion of connecting together the first and second polymer portions, the mandrel, when used, are removed from the lumen of tube formed by the first and second polymer portions. In one non-limiting method, the second polymer portion is connected to the first polymer portion by heat flowing or melting the second polymer portion to the first polymer portion while a mandrel is positioned in the lumen of the first polymer portion to maintain the diameter of the lumen of the first polymer portion at a desired size (e.g., maintain in the expanded orientation, etc.) to form a desired connection between the second polymer portion and the first polymer portion. An optional heat shrink layer (e.g., FEP or polyolefin) can be applied over a portion or all of the outer surface of the second polymer portion to apply a compressive force to second polymer portion during the melting or reflow of the second polymer portion or adhesive connection to the outer surface of the first polymer portion. Thereafter, the optional heat shrink layer can be removed from the outer surface of the second polymer portion. As can be appreciated, the first and second polymer portions can optionally be coextruded.

The one or more steps of the following process steps or other process steps as discussed above can be optionally used to form the expandable sheath: a) use and/or connection of an optional liner in the interior of the lumen of the expandable portion of the expandable sheath, b) use of an optional heat shrink layer to facilitate in the connecting of the optional liner to the polymer portion, c) folding one or more polymer portion into fully unexpanded position or orientation, d) applying an optional non-stick layer one or both sides of one or more polymer portions that are positioned between the overlapping ends of a portion of the expandable sheath so as to inhibit or prevent sticking, e) annealing of the expandable portion of the expandable sheath while the expandable portion of the expandable sheath is in the fully unexpanded position or orientation, f) use of optional heat shrink layer during annealing, g) using of an optional mandrel during the annealing process, h) optionally forming a taper end tip to the expandable portion of the expandable sheath, i) optionally adding an elastomeric continuous outer sleeve to A) at least a proximal portion of the expandable portion of the expandable sheath, B) at least a proximal portion of the expandable portion of the expandable sheath and the distal portion of the hub when the expandable sheath optionally includes a hub portion, C) 50-100% (and all values and ranges therebetween) of the longitudinal length of the expandable portion of the expandable sheath, and/or D) 50-100% (and all values and ranges therebetween) of the longitudinal length of the expandable portion of the expandable sheath and the distal portion of the hub when the expandable sheath optionally includes a hub portion, and/or j) optionally forming a spiral tip on a distal region of the expandable sheath or connecting a preformed spiral tip to the distal region of the expandable sheath.

The one or more steps of the above discussed process steps that were discussed above for the expandable sheath that was formed of a first polymer portion that has first and second thicknesses can also be used with the expandable sheath that has a first polymer portion of a first thickness and a second polymer portion of a second thickness and wherein the second polymer portion is connected to the first polymer portion. As such, these additional optional steps (e.g., a) use and/or connection of an optional liner in the interior of the lumen of the expandable portion of the expandable sheath, b) use of an optional heat shrink layer to facilitate in the connecting of the optional liner to the polymer portion, c) folding one or more polymer portion into fully unexpanded position or orientation, d) applying an optional non-stick layer one or both sides of one or more polymer portions that are positioned between the overlapping ends of a portion of the expandable sheath so as to inhibit or prevent sticking, e) annealing of the expandable portion of the expandable sheath while the expandable portion of the expandable sheath is in the fully unexpanded position or orientation, f) use of optional heat shrink layer during annealing, g) using of an optional mandrel during the annealing process, h) optionally forming a taper end tip to the expandable portion of the expandable sheath, i) optionally adding an elastomeric continuous outer sleeve to A) at least a proximal portion of the expandable portion of the expandable sheath, B) at least a proximal portion of the expandable portion of the expandable sheath and the distal portion of the hub when the expandable sheath optionally includes a hub portion, C) 50-100% (and all values and ranges therebetween) of the longitudinal length of the expandable portion of the expandable sheath, and/or D) 50-100% (and all values and ranges therebetween) of the longitudinal length of the expandable portion of the expandable sheath and the distal portion of the hub when the expandable sheath optionally includes a hub portion, and/or j) optionally forming a spiral tip on a distal region of the expandable sheath or connecting a preformed spiral tip to the distal region of the expandable sheath) will not be repeated in detail herein.

One non-limiting object of the disclosure, there is the provision of an expandable sheath.

Another and/or alternative non-limiting object of the disclosure is the provision of an expandable sheath that is usable with a medical device and which expandable sheath is expandable in at least the regions where the medical device is positioned in the expandable sheath and which sheath optionally partially or fully reforms to its same or similar size and shape after the medical device has passed through a portion or all of the expandable sheath.

Another and/or alternative non-limiting object of the disclosure is the provision of an expandable sheath that can be used to protect the body passageway of a patient as a medical device is inserted into and/or through the body passageway and to a treatment site.

Another and/or alternative non-limiting object of the disclosure is the provision of an expandable sheath that can be used with catheter arrangements for delivering a medical device to a treatment site so as facilitate in the delivery of a medical device in a body passageway.

Another and/or alternative non-limiting object of the disclosure is the provision of an expandable sheath that can be used with catheter arrangements for delivering a prosthetic valve to a treatment site, and thereby facilitating in the delivery of a catheter (e.g., balloon catheter, etc.) in a body passageway, and thus facilitating in the delivery of the prosthetic valve to the treatment site for the repairing and/or replacing of a heart valve, etc.

Another and/or alternative non-limiting object of the disclosure is the provision of an expandable sheath that can be used to reduce or minimize trauma to a body passageway (e.g., blood vessel, duct, etc.) by allowing for temporary expansion of a portion of the expandable sheath to accommodate at least a portion of the delivery system and/or medical device, followed by an optional return to the same or similar original diameter once the delivery system and/or medical device has partially or fully passes through the expandable sheath.

Another and/or alternative non-limiting object of the disclosure is the provision of an expandable sheath that can be configured to progressively increase in outer diameter and then optionally progressively decrease in outer diameter as the medical device moves through the interior passageway of the expandable sheath.

Another and/or alternative non-limiting object of the disclosure is the provision of an expandable sheath that can be configured such that the outer diameter or cross-sectional area of the expandable sheath and the diameter or cross-sectional area of the interior passageway expands to a second diameter along the longitudinal length of the expandable sheath as the medical device moves through the expandable sheath, and then optionally partially or fully returns to the first outer diameter once the medical device has fully passes through the expandable sheath.

Another and/or alternative non-limiting object of the disclosure is the provision of an expandable sheath that is absent a frame.

Another and/or alternative non-limiting object of the disclosure is the provision of an expandable sheath wherein the sheath includes one or more polymer portions that are formed of one or more materials selected from the group consisting of polyether-block-amide (PEBA) (e.g., Pebax™, etc.), polyvinyl acetate or polyvinyl acetate or poly(ethenyl ethanoate) (PVA or PVAC), polytetrafluoroethylene (PTFE), polyimide, polyetheretherketone (PEEK), polyurethane, nylon, polyethylene, polyamide, styrenic block copolymer (SBC) (e.g., polystyrene blocks and rubber blocks [e.g., polybutadiene, polyisoprene or their hydrogenated equivalents]), polyether block amides, polyether block ester copolymer, thermoset silicone, latex, poly-isoprene rubbers, high density polyethylene (HDPE), block copolymers made up of polyamide blocks and polyether blocks (e.g., Pebax™, etc.), polyurethane resin, elastomers formed of block copolymers made up of rigid polyamide blocks and soft polyether blocks.

Another and/or alternative non-limiting object of the disclosure is the provision of an expandable sheath wherein the expandable sheath is formed of a single polymer portion, and wherein the single polymer portion has a first and second portion that has respective first and second thickness that are different.

Another and/or alternative non-limiting object of the disclosure is the provision of an expandable sheath wherein the expandable sheath includes two polymer portions, and wherein the first polymer portion constitutes 5-49 wt. % of the total polymer in the expandable sheath having a first thickness, and the second polymer portion constitutes 51-95 wt. % of the total polymer in the expandable sheath that has a second thickness, and wherein the first and second thickness are different.

Another and/or alternative non-limiting object of the disclosure is the provision of an expandable sheath wherein the expandable sheath includes two polymer portions, and wherein the first and second polymer portions form a lumen, and wherein one end of the first polymer portion is connected to one end of the second polymer portion, and the other end of the first polymer portion is connected to the other end of the second polymer portion, and the first polymer portion optionally has an average thickness and/or durometer that is greater than an average thickness and/or durometer of the second polymer portion, and wherein the longitudinal length or circumferential length of the first polymer portion is optionally greater than the longitudinal length or circumferential length of the second polymer portion.

Another and/or alternative non-limiting object of the disclosure is the provision of an expandable sheath wherein the expandable sheath includes two polymer portions, and wherein the first and second polymer portions form a lumen, and wherein one end of the first polymer portion is connected to one end of the second polymer portion, and the other end of the first polymer portion is connected to the other end of the second polymer portion, and the first polymer portion optionally has an average thickness and/or durometer that is greater than an average thickness and/or durometer of the second polymer portion, and wherein the longitudinal length or circumferential length of the first polymer portion is optionally greater than the longitudinal length or circumferential length of the second polymer portion, and wherein a polymer liner is optionally inserted in at least a portion of the longitudinal length of the lumen, and wherein a stretchable polymer portion is optionally positioned on the outer surface of the first and second polymer portions.

Another and/or alternative non-limiting object of the disclosure is the provision of an expandable sheath wherein the expandable sheath is formed of a first polymer portion, and wherein the first polymer portion has a varying thickness about the circumference of the expandable sheath, and wherein a polymer liner is optionally inserted in at least a portion of the longitudinal length of the lumen, and wherein a second polymer portion is optionally positioned on the outer surface of the first polymer portion along at least a portion of the longitudinal length of the expandable sheath.

Another and/or alternative non-limiting object of the disclosure is the provision of an expandable sheath wherein the expandable sheath includes two polymer portions, and wherein the first and second polymer portions form a lumen, and wherein the first polymer portion has first and second ends that are not connected together and form a gap when the first polymer portion is in the fully expanded orientation, and wherein the second polymer portion is a continuous polymer layer that is positioned about the outer surface of the first polymer portion, and the first polymer portion optionally has an average thickness and/or durometer that is greater than an average thickness and/or durometer of the second polymer portion.

Another and/or alternative non-limiting object of the disclosure is the provision of an expandable sheath wherein the expandable sheath includes two polymer portions, and wherein the first and second polymer portions form a lumen, and wherein first polymer portion has first and second ends that are not connected together and form a gap when the first polymer portion is in the fully expanded orientation, and wherein the second polymer portion is a continuous polymer layer that is positioned about the outer surface of the first polymer portion, and the first polymer portion optionally has an average thickness and/or durometer that is greater than an average thickness and/or durometer of the second polymer portion, and wherein a polymer liner is optionally inserted in at least a portion of the longitudinal length of the lumen, and wherein a stretchable polymer portion is optionally positioned on the outer surface of the first and second polymer portions along at least a portion of the longitudinal length of the expandable sheath.

Another and/or alternative non-limiting object of the disclosure is the provision of an expandable sheath wherein the expandable sheath includes one or more radiopaque fillers or markers.

Another and/or alternative non-limiting object of the disclosure is the provision of an expandable sheath wherein the expandable sheath is at least partially or fully formed by an extrusion process, stamping process, reforming or heat process, 3D printer process, and/or casting process.

Another and/or alternative non-limiting object of the disclosure is the provision of a method of introducing a prosthetic valve, stent, catheter, and/or other type of medical device into a patient's vasculature, the method comprising: a) optionally forming an incision in a patient; b) inserting an expandable sheath through the optional incision and at least partially into a blood vessel of a patient; the expandable sheath is expandable from a first diameter to a second diameter; the second diameter is greater than the first diameter; the expandable sheath includes an internal cavity that extends a longitudinal length of the expandable sheath; the expandable sheath includes one or more polymer portions; and c) advancing the prosthetic valve, stent, catheter, and/or other type of medical device through the internal cavity of the expandable sheath to cause the expandable sheath to expand from the first diameter to the second diameter as the prosthetic valve, stent, catheter, and/or other type of medical device partially or fully passes through the internal cavity of the expandable sheath.

Another and/or alternative non-limiting object of the disclosure is the provision of method of introducing a prosthetic valve into and through the expandable sheath, and into a patient's vasculature, and wherein the prosthetic valve is a prosthetic valve mounted in a radially crimped state on a delivery apparatus.

Another and/or alternative non-limiting object of the disclosure is the provision of method of introducing a prosthetic valve, stent, catheter, and/or other type of medical device into a patient's vasculature, and wherein the expandable sheath contracts from the second diameter to the first diameter after the prosthetic valve, stent, catheter, and/or other type of medical device has partially or fully passed through the internal cavity of the expandable sheath.

Another and/or alternative non-limiting object of the disclosure is the provision of a method of introducing a prosthetic valve, stent, catheter, and/or other type of medical device into a patient's vasculature, the method comprising: a) inserting an expandable sheath at least partially into a blood vessel of a patient; and b) advancing the prosthetic valve, stent, catheter, and/or other type of medical device through a central lumen of the expandable sheath to cause the expandable sheath to expand from an outwardly directed radial force of the prosthetic valve, stent, catheter, and/or other type of medical device exerted against the expandable sheath, and then the expandable sheath at least partially contracts as the prosthetic valve, stent, catheter, and/or other type of medical device passes through the expandable sheath.

Another and/or alternative non-limiting object of the disclosure is the provision of an expandable sheath wherein an outer surface of the outer layer portion includes a friction-reducing coating or layer; the friction-reducing coating or layer optionally has a static coefficient of friction of no more than 0.1.

Another and/or alternative non-limiting object of the disclosure is the provision of an expandable sheath wherein an inner surface of the lumen of the expandable sheath includes a friction-reducing coating or layer; the friction-reducing coating or layer optionally has a static coefficient of friction of no more than 0.1.

Another and/or alternative non-limiting object of the disclosure is the provision of an expandable sheath wherein two or more polymer layer portions are subjected to a reflow process to connect together the two or more polymer layer portions.

Another and/or alternative non-limiting object of the disclosure is the provision of an expandable sheath that optionally includes a tapered or conical-shaped front portion that tapers in size from the front end of the expandable sheath; and wherein the tapered or conical-shaped front portion includes an opening therethrough to facilitate in the insertion of the medical device through the tapered or conical-shaped front portion and into the body of the expandable sheath.

Another and/or alternative non-limiting object of the disclosure is the provision of an expandable sheath that optionally includes one or more breakable connection points. The one or more optional breakable connection points can be located a) on the outer layer of the expandable sheath, and wherein the one or more optional breakable connection points can be used to maintain the expandable sheath in the collapsed or unexpanded position until a) a medical device is inserted through the internal passageway or cavity of the tubular-shaped portion of the expandable sheath to cause the expandable sheath to move to the non-collapsed or expanded position to cause breakage one the one or more breakable connection points, or b) a medical device is inserted through the internal passageway or cavity of the tubular-shaped portion of the expandable sheath to cause the expandable sheath to move to the non-collapsed or expanded position to cause breakage one of the one or more breakable connection points.

Another and/or alternative non-limiting object of the disclosure is the provision of a method of introducing a medical device into a body passageway of a patient wherein the step of inserting includes passing the expandable sheath transcutaneously through a surgically-created opening in the patient's skin such that at least a portion of the outer payer portion of the sheath is positioned adjacent to the surgically-created opening.

Another and/or alternative non-limiting object of the disclosure is the provision of a method of introducing a medical device into a body passageway of a patient further including the step of implanting the medical device at a treatment site within the patient.

Another and/or alternative non-limiting object of the disclosure is the provision of a method of introducing a medical device into a body passageway of a patient further including the step of expanding the medical device at the treatment site of the patient.

Another and/or alternative non-limiting object of the disclosure is the provision of an expandable sheath that provides access to a body passageway to facilitate in delivering a medical device into the body passageway wherein the tubular structure is subjected to annealing to so that the tubular structure remains in the unexpanded orientation when no forces are applied to the tubular structure.

Another and/or alternative non-limiting object of the disclosure is the provision of an expandable sheath that provides access to a body passageway to facilitate in delivering a medical device into the body passageway wherein the expandable sheath portion includes an internal liner; the internal liner is positioned in the internal cavity of the tubular structure along 50-100% of the longitudinal length of the tubular structure; the internal liner is formed of a different material from a material used to form the tubular structure.

Another and/or alternative non-limiting object of the disclosure is the provision of an expandable sheath that provides access to a body passageway to facilitate in delivering a medical device into the body passageway wherein the internal liner has a melting point that is greater than the material used to form the tubular structure.

Another and/or alternative non-limiting object of the disclosure is the provision of an expandable sheath that provides access to a body passageway to facilitate in delivering a medical device into the body passageway wherein the internal liner has a thickness that is less than a thickness of the thick portion of the tubular structure.

Another and/or alternative non-limiting object of the disclosure is the provision of an expandable sheath that provides access to a body passageway to facilitate in delivering a medical device into the body passageway wherein the internal liner has a static coefficient of friction that is less than a static coefficient of friction of the material used to form the tubular structure.

Another and/or alternative non-limiting object of the disclosure is the provision of an expandable sheath that provides access to a body passageway to facilitate in delivering a medical device into the body passageway wherein an outer surface of the internal liner includes etching; the etching is used to facilitate is connecting the internal liner to the tubular structure.

Another and/or alternative non-limiting object of the disclosure is the provision of an expandable sheath that provides access to a body passageway to facilitate in delivering a medical device into the body passageway further including hub connection portion; a distal end of the hub connection portion is connected to a proximal end of the expandable sheath portion; the hub connection portion is formed of a less flexible material than a material used to form the expandable sheath portion.

Another and/or alternative non-limiting object of the disclosure is the provision of a method wherein the internal liner includes one or more properties selected from the group consisting of a) the internal liner has a melting point that is greater than the material used to form the tubular structure, b) the internal liner has a thickness that is less than a thickness of the thick portion of the tubular structure, c) the internal liner has a static coefficient of friction that is less than a static coefficient of friction of the material used to form the tubular structure, d) an outer surface of the internal liner includes etching, and wherein the etching is used to facilitate is connecting the internal liner to the tubular structure, and/or e) the internal liner is connected to the interior surface of the internal cavity of the tubular structure by adhesive and/or a connection process that includes the use of heat and/or pressure.

Another and/or alternative non-limiting object of the disclosure is the provision of a method further including the steps of a) folding the tubular structure to form the tubular structure in the unexpanded orientation, and b) annealing the tubular structure while the tubular structure is maintained in the unexpanded orientation.

Another and/or alternative non-limiting object of the disclosure is the provision of a method wherein an annealing temperature is less than a melting point temperature of the material used to form the tubular structure.

Another and/or alternative non-limiting object of the disclosure is the provision of a method further including the step of applying a non-stick layer between a portion of a region where the first end portion and a second end portion of the thick portion overlie one another when the tubular structure is in the unexpanded orientation; the non-stick layer a) formed of different material from the material used to form the tubular structure, and/or b) has a higher melting point than the material used to form the tubular structure.

Another and/or alternative non-limiting object of the disclosure is the provision of a method further including the step of forming a taper end tip at a distal end portion of the tubular structure.

Another and/or alternative non-limiting object of the disclosure is the provision of a method further including the step of providing a connection hub portion and connecting the expandable sheath portion to the connection hub portion; the connection hub portion is formed of a material that is less flexible than the material used to form the tubular structure; a distal end of the hub connection portion is connected to a proximal end of the expandable sheath portion.

Another and/or alternative non-limiting object of the disclosure is the provision of a method further including the step of applying an elastomeric continuous outer sleeve over the connection between the hub connection portion and the expandable sheath portion; the elastomeric continuous outer sleeve has a melting point that is less than a melting point of the hub connection portion and/or the expandable sheath portion.

Another and/or alternative non-limiting object of the disclosure is the provision of a method wherein the step of forming further includes forming a tubular structure that is absent the first end portion and the second end portion, and removing a portion of the tubular structure along at least a portion of the longitudinal length of the tubular structure for form the first end portion and the second end portion and a gap between the first end portion and the second end portion.

Another and/or alternative non-limiting object of the disclosure is the provision of an expandable sheath can optionally include a specially cut tip region at the distal end or region of the expandable sheath to facilitate in the operation of the expandable sheath.

Another and/or alternative non-limiting object of the disclosure is the provision of an expandable sheath can optionally include a specially cut tip region at the distal end or region of the expandable sheath to facilitate in the operation of the expandable sheath, wherein the cut region has a spiral-type cut is formed in the distal most tip region of an expandable sheath. Such spiral cut is used to overcome and/or address the issues of a) the catheter being deflected away from being coaxial with the longitudinal axis of the expandable sheath as the catheter is move out from the distal end of the expandable sheath, and/or b) the tip region of the expandable sheath being caused to move away from being coaxial with the longitudinal axis of the expandable sheath as the catheter moves out from the distal end of the sheath.

Another and/or alternative non-limiting object of the disclosure is the provision of an expandable sheath can optionally include a specially cut tip region at the distal end or region of the expandable sheath to facilitate in the operation of the expandable sheath, wherein the cut region has a spiral-type cut is formed in the distal most tip region of an expandable sheath, and wherein the spiral cut is a) directly formed in a distal region of the expandable sheath, or b) form in a separate component that is connected (e.g., adhesive connection, melted connection, friction connection, weld or soldered connection, etc.) to the distal end or distal end region of the main body of the expandable sheath.

Another and/or alternative non-limiting object of the disclosure is the provision of an expandable sheath that provides access to a body passageway to facilitate in delivering a medical device into the body passageway; the expandable sheath includes an expandable sheath portion that is expandable from an unexpanded orientation having a first cross-sectional area to an expanded orientation having a second cross-sectional area; the second cross-sectional area is greater than the first cross-sectional area; the expandable sheath portion includes a tubular structure along 80-100% of the longitudinal length of the expandable sheath portion; the tubular structure is at least partially formed of a first polymer; the expandable sheath portion includes an internal cavity that extends at least 25% of a longitudinal length of the expandable sheath portion; the tubular structure includes a thick portion and a thin portion; an average thickness of the thick portion is greater than an average thickness of the thin portion; the thick portion includes first and second ends; the thin portion is connected to and/or overlies the first and second ends; a cross-sectional shape of the expandable sheath portion that is normal to a longitudinal axis of the tubular structure includes a thick portion positioned about 30-90% of the internal cavity of the expandable sheath portion when the expandable sheath portion is in the expanded orientation; the thin portion positioned about 10-70% of the internal cavity of the expandable sheath portion when the expandable sheath portion is in the expanded orientation; the cross-sectional shape of the tubular structure is constant along 25-100% of the longitudinal length of the tubular structure when the expandable sheath portion is in the expanded orientation; a first end portion and a second end portion of the thick portion overlie one another such that a portion of an outer perimeter of the tubular structure includes overlying the first and second end portions when the when the expandable sheath portion is in the unexpanded orientation; the first end portion and the second end portion of the tubular structure are spaced apart from one another when the when the expandable sheath portion is in the expanded orientation; and wherein the expandable sheath portion is absent a frame; and wherein 50-100% of a longitudinal length of the thin portion is positioned between overlapping regions of the first end portion and the second end portion when the expandable sheath portion is in the unexpanded orientation; and wherein the expandable sheath portion is configured to expand to the second cross-sectional area during movement of the medical device through the internal cavity of the tubular structure; and wherein the expandable sheath portion is configured to contract from the second cross-sectional area after the medical device partially or fully passes through the internal cavity of the tubular structure.

Another and/or alternative non-limiting object of the disclosure is the provision of an expandable sheath wherein the tubular structure is formed of a single thick portion and a single thin portion.

Another and/or alternative non-limiting object of the disclosure is the provision of an expandable sheath wherein the thick portion and the thin portion are formed by the first polymer, and wherein there is no connection mechanism used between the thick portion and the thin portion.

Another and/or alternative non-limiting object of the disclosure is the provision of an expandable sheath wherein the thick portion is formed by the first polymer and the thin portion is formed by a second polymer; the thick portion includes first and second ends; the thin portion includes first and second ends; the first ends of the thick and thin portions are connected together; the second ends of the thick and thin portions are connected together; the thick portion is connected to the thin portion by one or more connection mechanisms.

Another and/or alternative non-limiting object of the disclosure is the provision of an expandable sheath wherein the thick portion is formed by the first polymer and the thin portion is formed by a second polymer; the thick portion includes first and second ends; the thin portion is formed in a continuous layer; the thin portion is connected to one or more portions of an outer surface of the thick portion; the thin portion overlies 50-100% of the outer surface of the thick portion; a portion of the thin portion spans a gap between the first and second ends of the thick portions when the expandable sheath portion is in the expanded orientation; the thin portion is positioned along 50-100% of a longitudinal length of the thick portion.

Another and/or alternative non-limiting object of the disclosure is the provision of an expandable sheath wherein the connection mechanism includes one of more of an adhesive, a heat created connection, a melted connection, a pressure connection, and/or a mechanical connection.

Another and/or alternative non-limiting object of the disclosure is the provision of an expandable sheath wherein the first and second polymers are a) formed of the same type of polymer material or b) formed of a different polymer material.

Another and/or alternative non-limiting object of the disclosure is the provision of an expandable sheath wherein an average durometer of material used to form the thick portion is greater than an average durometer of material used to form the thin portion.

Another and/or alternative non-limiting object of the disclosure is the provision of an expandable sheath wherein 80-100% of a longitudinal length of the thick portion of the tubular structure is flexible, but is not foldable; 5-100% of a longitudinal length of the thin portion is both flexible and foldable.

Another and/or alternative non-limiting object of the disclosure is the provision of an expandable sheath wherein the tubular structure is subjected to annealing so that the tubular structure remains in the unexpanded orientation when no forces are applied to the tubular structure.

Another and/or alternative non-limiting object of the disclosure is the provision of an expandable sheath wherein the expandable sheath portion includes an internal liner; the internal liner is positioned in the internal cavity of the tubular structure along 50-100% of the longitudinal length of the tubular structure; the internal liner is formed of a different material from the material used to form the tubular structure.

Another and/or alternative non-limiting object of the disclosure is the provision of an expandable sheath wherein the internal liner has a melting point that is greater than the material used to form the tubular structure.

Another and/or alternative non-limiting object of the disclosure is the provision of an expandable sheath wherein the internal liner has a thickness that is less than a thickness of the thick portion of the tubular structure.

Another and/or alternative non-limiting object of the disclosure is the provision of an expandable sheath wherein the internal liner has a static coefficient of friction that is less than a static coefficient of friction of the material used to form the tubular structure.

Another and/or alternative non-limiting object of the disclosure is the provision of an expandable sheath wherein an outer surface of the internal liner includes etching; the etching is used to facilitate is connecting the internal liner to the tubular structure.

Another and/or alternative non-limiting object of the disclosure is the provision of an expandable sheath further including a hub connection portion; a distal end of the hub connection portion is connected to a proximal end of the expandable sheath portion; the hub connection portion is formed of a material that a) has a higher durometer than a durometer of the thick portion of the tubular structure, and/or b) is less flexible material than the material used to form the thick portion of the tubular structure.

Another and/or alternative non-limiting object of the disclosure is the provision of an expandable sheath wherein a distal end region of the expandable sheath portion includes a spiral cut.

Another and/or alternative non-limiting object of the disclosure is the provision of an expandable sheath wherein a distal end region of the expandable sheath portion includes a taper region.

Another and/or alternative non-limiting object of the disclosure is the provision of an expandable sheath wherein the distal end region is a separate component from the tubular structure; the distal end region is connected to the tubular structure by one or more connection mechanisms that include an adhesive, a heat created connection, a melted connection, a pressure connection, and/or a mechanical connection.

Another and/or alternative non-limiting object of the disclosure is the provision of an expandable sheath wherein a distal end region is formed of a different material from the tubular structure.

Another and/or alternative non-limiting object of the disclosure is the provision of a method of using an expandable sheath to insert a medical device into a patient; the method comprising: a) providing an expandable sheath as disclosed above; b) inserting a portion of the expandable sheath portion into the body passageway; and c) inserting the medical device through the expandable sheath portion and into the body passageway; and wherein the expandable sheath portion expands to the second cross-sectional area during movement of the medical device through the internal cavity of the tubular structure; and wherein the expandable sheath portion contracts from the second cross-sectional area after the medical device partially or fully passes through the internal cavity of the tubular structure.

Another and/or alternative non-limiting object of the disclosure is the provision of a method of using an expandable sheath wherein the medical device is a prosthetic heart valve, a stent, angioplasty balloon, or LAA (left atrial appendage) closure device.

Another and/or alternative non-limiting object of the disclosure is the provision of a method of forming at least a portion of an expandable sheath; the method comprising: a) forming an expandable sheath portion of the expandable sheath; the step of forming the expandable sheath portion of the expandable sheath includes forming a tubular structure of the expandable portion using a first polymer; the step of forming includes a process selected from the group consisting of extrusion, molding and/or 3D printing; the expandable sheath portion includes a tubular structure along 80-100% of the longitudinal length of the expandable sheath portion; the tubular structure is at least partially formed of a first polymer; the expandable sheath portion includes an internal cavity that extends at least 25% of a longitudinal length of the expandable sheath portion; the tubular structure includes a thick portion and a thin portion; an average thickness of the thick portion is greater than an average thickness of the thin portion; the thick portion includes first and second ends; the thin portion is connected to and/or overlies the first and second ends of the thick portion; a cross-sectional shape of the expandable sheath portion that is normal to a longitudinal axis of the tubular structure includes a thick portion positioned about 30-90% of the internal cavity of the expandable sheath portion when the expandable sheath portion is in the expanded orientation; the thin portion is positioned about 10-70% of the internal cavity of the expandable sheath portion when the expandable sheath portion is in the expanded orientation; the cross-sectional shape of the tubular structure is constant along 25-100% of the longitudinal length of the tubular structure when the expandable sheath portion is in the expanded orientation; a first end portion and a second end portion of the thick portion overlie one another such that a portion of an outer perimeter of the tubular structure includes overlying the first and second end portions when the when the expandable sheath portion is in the unexpanded orientation; the first end portion and the second end portion of the tubular structure are spaced apart from one another when the when the expandable sheath portion is in the expanded orientation; and b) connecting a proximal end of the expandable sheath portion to a distal end of a hub connection portion; the hub connection portion is formed of a less flexible material than a material used to form the expandable sheath portion.

Another and/or alternative non-limiting object of the disclosure is the provision of a method further including the step of connecting an interior liner to an interior surface of the internal cavity of the tubular structure; the internal liner is positioned in the internal cavity of the tubular structure along 50-100% of the longitudinal length of the tubular structure; the internal liner is formed of a different material from a material used to form the tubular structure.

Another and/or alternative non-limiting object of the disclosure is the provision of a method wherein the internal liner includes one or more properties selected from the group consisting of a) the internal liner has a melting point that is greater than the material used to form the tubular structure, b) the internal liner has a thickness that is less than a thickness of the thick portion of the tubular structure, c) the internal liner has a static coefficient of friction that is less than a static coefficient of friction of the material used to form the tubular structure, d) an outer surface of the internal liner includes etching, and wherein the etching is used to facilitate is connecting the internal liner to the tubular structure, and/or e) the internal liner is connected to the interior surface of the internal cavity of the tubular structure by adhesive and/or a connection process that includes the use of heat and/or pressure.

Another and/or alternative non-limiting object of the disclosure is the provision of a method further including the steps of a) folding the tubular structure to form the tubular structure in the unexpanded orientation, and b) annealing the tubular structure while the tubular structure is maintained in the unexpanded orientation; an annealing temperature is less than a melting point temperature of the material used to form the tubular structure.

Another and/or alternative non-limiting object of the disclosure is the provision of a method further including the step of applying a non-stick layer between a portion of a region where the first end portion and the second end portion of the thick portion overlie one another when the tubular structure is in the unexpanded orientation; the non-stick layer a) is formed of different material from the material used to form the tubular structure, and/or b) has a higher melting point than the material used to form the tubular structure.

Another and/or alternative non-limiting object of the disclosure is the provision of a method further including the step of forming a taper end tip at a distal end portion of the expandable sheath portion.

Another and/or alternative non-limiting object of the disclosure is the provision of a method further including the step of forming a spiral cut a distal end portion of the expandable sheath portion.

Another and/or alternative non-limiting object of the disclosure is the provision of a method further including the step of applying an elastomeric continuous outer sleeve over a connection between the hub connection portion and the expandable sheath portion; the elastomeric continuous outer sleeve has a melting point that is less than a melting point of the hub connection portion and/or the expandable sheath portion.

Another and/or alternative non-limiting object of the disclosure is the provision of a method wherein the thick portion and the thin portion are formed by the first polymer; and wherein there is no connection mechanism used between the thick portion and the thin portion.

Another and/or alternative non-limiting object of the disclosure is the provision of a method wherein the thick portion and the thin portion are formed by an extrusion process or a molding process.

Another and/or alternative non-limiting object of the disclosure is the provision of a method wherein the thick portion is formed by the first polymer and the thin portion is formed by a second polymer; the thick portion includes first and second ends; the thin portion includes first and second ends; and further including the step of connecting together of the first ends of the thick and thin portions and the second ends of the thick and thin portions by one or more connection mechanisms.

Another and/or alternative non-limiting object of the disclosure is the provision of a method wherein the thick portion is formed by the first polymer and the thin portion is formed by a second polymer; the thick portion includes first and second ends; the thin portion is formed in a continuous layer; and further including the step of connecting the thin portion to one or more portions of an outer surface of the thick portion by one or more connection mechanisms; the thin portion overlies 50-100% of the outer surface of the thick portion; a portion of the thin portion spans a gap between the first and second ends of the thick portions when the expandable sheath portion is in the expanded orientation; the thin portion is positioned along 50-100% of a longitudinal length of the thick portion.

Another and/or alternative non-limiting object of the disclosure is the provision of a method wherein the connection mechanism includes one of more of an adhesive, a heat created connection, a melted connection, a pressure connection, and/or a mechanical connection.

Another and/or alternative non-limiting object of the disclosure is the provision of a method wherein the first and second polymers are a) formed of the same type of polymer material or b) formed of a different polymer material.

Another and/or alternative non-limiting object of the disclosure is the provision of a method wherein the thick portion that formed of the first polymer is formed by a) an extrusion process or a molding process that forms the final shape of the thick portion, or b) an extrusion process or a molding process that forms a tubular structure and thereafter cutting a slot along a portion or all of a longitudinal length of the tubular structure.

Another and/or alternative non-limiting object of the disclosure is the provision of a method wherein an average durometer of material used to form the thick portion is greater than an average durometer of material used to form the thin portion.

Another and/or alternative non-limiting object of the disclosure is the provision of a method wherein 80-100% of a longitudinal length of the thick portion of the tubular structure is flexible, but is not foldable; 5-100% of a longitudinal length of the thin portion is both flexible and foldable.

Another and/or alternative non-limiting object of the disclosure is the provision of a method wherein the tubular structure is subjected to annealing to so that the tubular structure remains in the unexpanded orientation when no forces are applied to the tubular structure.

Other aspects, advantages and novel features of the present disclosure will become apparent from the following detailed description and figures of the disclosure.

### BRIEF DESCRIPTION OF THE DRAWINGS

Non-limiting and non-exhaustive embodiments are described with reference to the following drawings, wherein like labels refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. Reference may now be made to the drawings, which illustrate various embodiments that the disclosure may take in physical form and in certain parts and arrangement of parts wherein:

FIG. 1 is a side view of a non-limiting delivery system and non-limiting medical device and a proximal region of an expandable sheath in accordance with the present disclosure.

FIG. 2A is a front isometric view of a non-limiting expandable sheath in the unexpanded orientation in accordance with the present disclosure.

FIG. 2B is a front isometric view of a non-limiting expandable sheath in the expanded orientation in accordance with the present disclosure.

FIG. 3A is a front isometric view of the expandable portion of a non-limiting expandable sheath in the unexpanded orientation in accordance with the present disclosure.

FIG. 3B is a front isometric view of the expandable portion of a non-limiting expandable sheath in the expanded orientation in accordance with the present disclosure.

FIG. 4 is a top plan view of the expandable sheath of FIG. 2A.

FIG. 5 is top plan view of the expandable sheath of FIG. 2B.

FIGS. 6A-6E are different cross-sectional views of different non-limiting embodiments of the expandable sheath along line 6A-E-6A-E of FIG. 4.

FIGS. 7A-7E are different cross-sectional views of different non-limiting embodiments of the expandable sheath along line 7A-E-7A-E of FIG. 5.

FIG. 7F is a cross-section view of a non-limiting embodiment of the expandable sheath and illustrates the optional use of temporary non-stick layer.

FIG. 8 is perspective view of the expandable sheath portion that includes one or more break points.

FIG. 9 is a photograph of a distal portion of a prior art expandable sheath and a portion of a prior art catheter extending outwardly from the distal end of the prior art expandable sheath and wherein a portion of the distal end of the prior art expandable sheath is being deflected outwardly from the catheter.

FIG. 10 is a side view of a distal end region of the expandable sheath in accordance with the present disclosure which illustrates a non-limiting spiral cut in the distal end region of the expandable sheath.

FIG. 11 is a photograph a distal end region of the expandable sheath in accordance with the present disclosure which illustrates a portion of a prior art catheter extending through a spiral cut in the distal end region of the expandable sheath in accordance with the present disclosure.

It is understood that all dimensions set forth in the drawings represent one non-limiting dimension of the expandable sheath in accordance with the present disclosure, thus other dimensions of the expandable sheath can be used in accordance with the present disclosure.

### DESCRIPTION OF NON-LIMITING EMBODIMENTS OF THE DISCLOSURE

A more complete understanding of the articles/devices, processes and components disclosed herein can be obtained by reference to the accompanying drawings. These figures are merely schematic representations based on convenience and the ease of demonstrating the present disclosure, and are, therefore, not intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any unavoidable impurities that might result therefrom, and excludes other ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values and all intermediate ranges).

It is understood that all ranges contained in the disclosure include the endpoints of the ranges and all intermediate values and intermediate ranges.

The terms “about” and “approximately” can be used to include any numerical value that can vary without changing the basic function of that value. When used with a range, “about” and “approximately” also disclose the range defined by the absolute values of the two endpoints, e.g., “about 2 to about 4” also discloses the range “from 2 to 4.” Generally, the terms “about” and “approximately” may refer to plus or minus 10% of the indicated number.

Percentages of elements should be assumed to be percent by weight of the stated element, unless expressly stated otherwise.

Although the operations of exemplary embodiments of the disclosed method may be described in a particular, sequential order for convenient presentation, it should be understood that disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed.

For the sake of simplicity, the attached figures may not show the various ways (readily discernable, based on this disclosure, by one of ordinary skill in the art) in which the disclosed system, method and apparatus can be used in combination with other systems, methods and apparatuses. Additionally, the description sometimes uses terms such as “produce” and “provide” to describe the disclosed method. These terms are abstractions of the actual operations that can be performed. The actual operations that correspond to these terms can vary depending on the particular implementation and are, based on this disclosure, readily discernible by one of ordinary skill in the art.

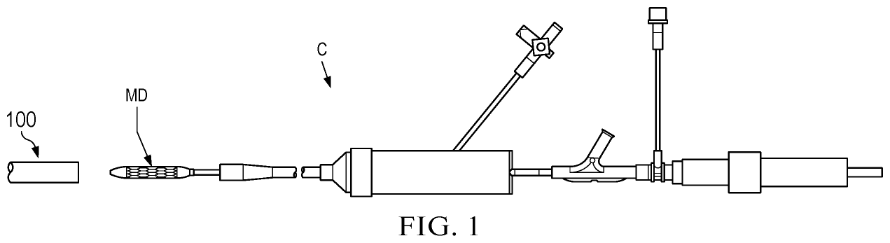

Referring now to FIG. 1, there is illustrate a proximal portion of an expandable sheath 100 in accordance with the present disclosure that can be used with a representative medical device delivery apparatus C for delivering a medical device (such as a TAV or other type of prosthetic valve) MD to the heart of a patient; however, it will be appreciated that the medical device can be other types of devices (e.g., stent, balloon catheter, LAA (left atrial appendage) closure devices. etc.). The delivery apparatus C can optionally include a steerable guide catheter (also referred to as a flex catheter), a balloon catheter, and/or an optional nose catheter. The guide catheter, the balloon catheter, and the nose catheter can be configured to slide longitudinally relative to each other to facilitate in the delivery and positioning of the medical device at a treatment site in the patient. Expandable sheath 100 can be inserted into a blood vessel such that one end of the expandable sheath is inserted into the blood vessel and the other end extends outside the body of a patient. Expandable sheath 100 can optionally include a valve (e.g., hemostasis valve) (not shown) at the proximal end of the expandable sheath. A portion of the delivery apparatus C that includes the medical device MD can be inserted into and through the expandable sheath 100 and into the blood vessel, and the medical device MD can then be delivered and implanted within a patient at the treatment site in the patient.

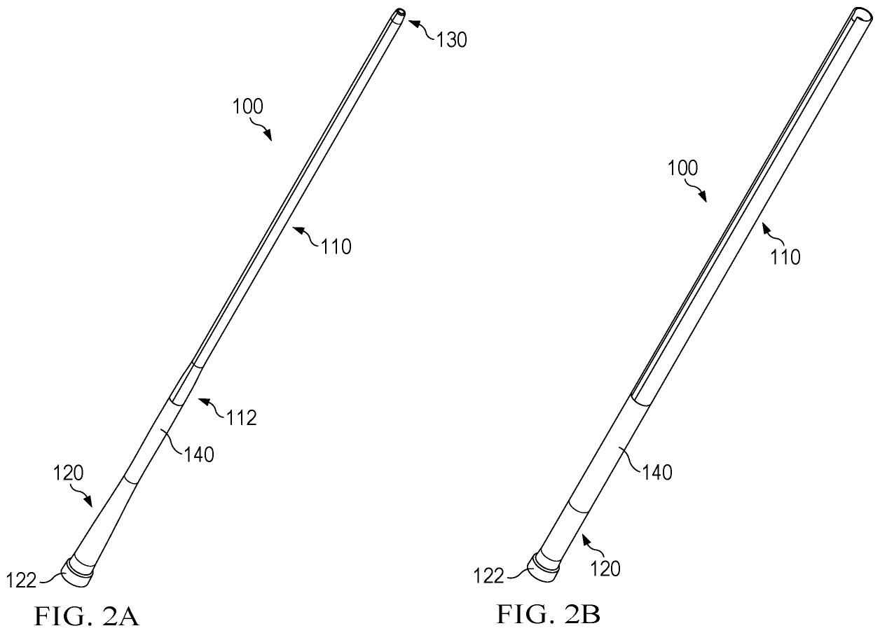

Referring now to FIGS. 2-7E, 9 and 10, several non-limiting embodiments of expandable sheath 100 in accordance with the present disclosure are illustrated.

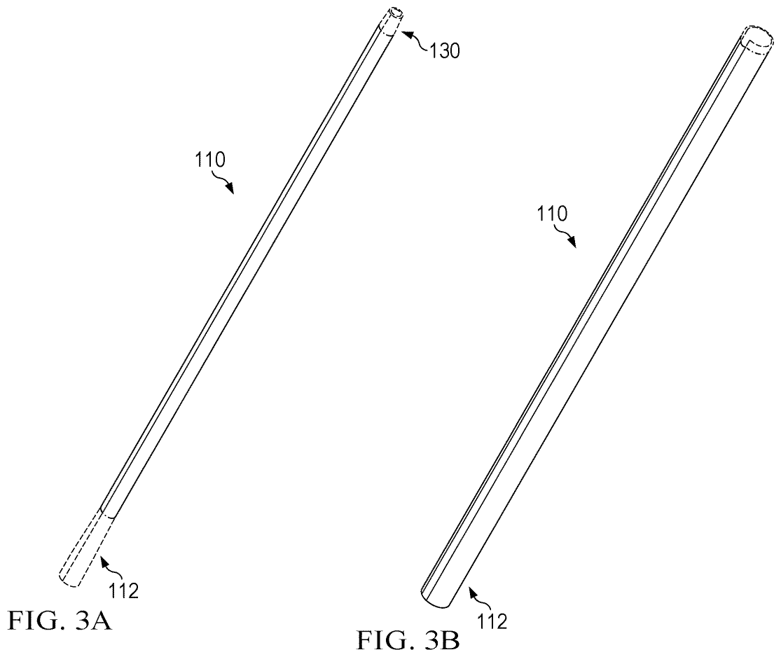

Referring now to FIG. 2, there is illustrated a front isometric view of a non-limiting expandable sheath 100 in an unexpanded orientation. Referring now to FIG. 3, there is illustrated a front isometric view of a non-limiting expandable sheath 100 in an expanded orientation.

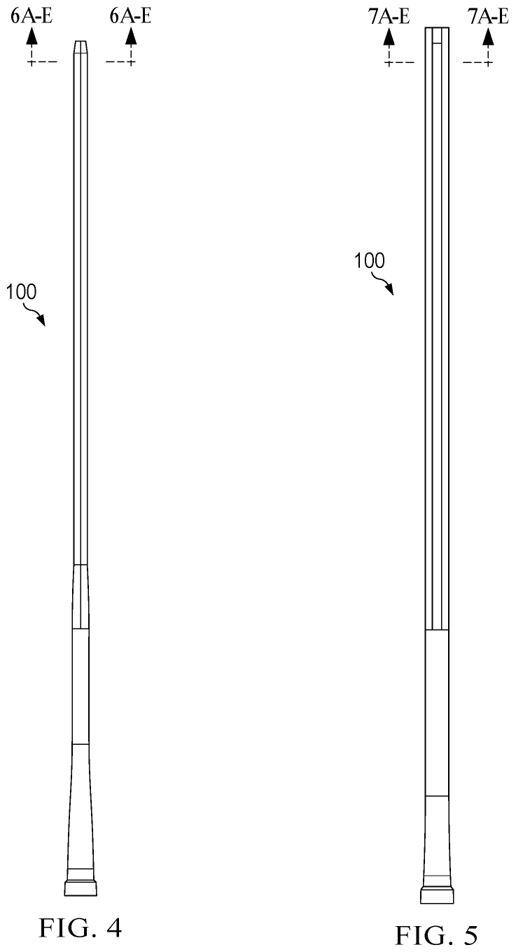

Referring now to FIG. 4, there is illustrated a top plan view of the expandable sheath of FIG. 2.

Referring now to FIG. 5, there is illustrated a top plan view of the expandable sheath of FIG. 3.

The expandable sheath 100 illustrated in FIGS. 2-5 includes an expandable sheath portion 110, a proximal sheath portion or hub portion 120, and optional tapered end portion 130 at the end region of the expandable sheath portion 110, and an optional elastomeric outer sleeve 140 that is positioned over the region between the connection between the expandable sheath portion 110 and the proximal sheath portion 120. The proximal end 122 of the proximal sheath portion 120 is configured to receive a distal portion of a the delivery apparatus C that includes the medical device MD. As can be appreciated, other medical device can be inserted into the proximal end 122 of the proximal sheath portion 120 (e.g., a medical device inserted into the lumen of the expandable medical device to break open breakable connection points on the expandable sheath, etc.).

The distal end or distal end portion of the proximal sheath portion 120 is configured to be connected to proximal end or proximal end portion of the expandable sheath portion 110. The type of connection is non-limiting (e.g., adhesive connection, melted connection, mechanical connection, etc.). An optional outer sleeve 140 can be positioned over the region between the connection between the expandable sheath portion 110 and the proximal sheath portion 120 to a) strengthen and/or facilitate in the connection between the distal end or distal end portion of the proximal sheath portion 120 and the proximal end or proximal end portion of the expandable sheath portion 110, and/or b) reduce or eliminate leakage of liquid at the connection between the distal end or distal end portion of the proximal sheath portion 120 and the proximal end or proximal end portion of the expandable sheath portion 110. The optional outer sleeve 140 is generally a polymer material, and can optionally be a heat shrinkable material, a flexible material, a stretchable material, and/or a foldable material. The optional outer sleeve 140 can be connected to the expandable sheath portion 110 and/or the proximal sheath portion 120 various arrangements (e.g., adhesive connection, melted connection, mechanical connection, heat shrink connection, etc.). Generally, the material used to form the outer sleeve 140 is different from one or both of the expandable sheath portion 110 and/or the proximal sheath portion 120; however, this is not required. Generally, the average thickness of the optional outer sleeve 140 is less than the average thickness of the proximal sheath portion 120. In another non-limiting embodiment, the outer sleeve 140 constitutes a portion of the proximal sheath portion 120 and may be a separate piece or form a single piece with the proximal sheath portion. In such a non-limiting configuration, the outer sleeve 140 can be tapered and have a fixed, non-expandable shape. The diameter and/or cross-sectional area of the lumen in the optional outer sleeve 140 can a) be constant along the longitudinal length of the outer sleeve, or b) have a constant or non-constant reduction in diameter from the proximal end to the distal end of the outer sleeve. The diameter and/or cross-sectional area of the lumen in the optional outer sleeve 140 can be fixed or can be expandable as the medical device MID and/or catheter shaft CS passes through the lumen of the optional outer sleeve 140.

The outer cross-sectional area and/or the lumen of the proximal sheath portion 120 can optionally taper-down in cross-sectional size one or more times between the proximal end 122 and distal end of the proximal sheath portion 120. The cross-sectional size or diameter of the opening (e.g., circular opening, oval opening, etc.) at the distal end 124 of the proximal sheath portion 120 is generally the same or within ±10% (and all values and ranges therebetween) the cross-sectional size or diameter of the opening (e.g., circular opening, oval opening, etc.) at the proximal end 112 of the expandable sheath portion 110 when the proximal end 112 of the expandable sheath portion 110 is in the fully expanded or open orientation. The faces of the distal end 124 of the proximal sheath portion 120 and the proximal end 112 or proximal end portion of the expandable sheath portion 110 can be connected together by one or more arrangements (e.g., adhesive connection, connection involving pressure and/or heat, mechanical connection, etc.), b) a portion of the distal portion of the proximal sheath portion 120 can telescopically receive a portion of the proximal portion of the expandable sheath portion 110 and be connected together by one or more arrangement (e.g., adhesive connection, connection involving pressure and/or heat, friction connection, mechanical connection, heat created connection, etc.), or c) a portion of the distal portion of the proximal sheath portion 120 can be telescopically inserted into a portion of the proximal portion of the expandable sheath portion 110 and be connected together one or more arrangements (e.g., adhesive connection, connection involving pressure and/or heat, friction connection, heat created connection, mechanical connection, etc.). As can be appreciated, other connection arrangements can be used.

The proximal sheath portion 120 is generally partially or fully formed of a hard, generally inflexible material (e.g., hard plastic, metal, etc.). In one non-limiting embodiment, the proximal sheath portion 120 is a non-expandable component that retains its shape. In another non-limiting embodiment, at least a portion of the proximal sheath portion 120 is expandable. As illustrated in FIG. 2B, the distal portion of the proximal sheath portion 120 is illustrated as being expandable; however, this is not required.

Generally, the proximal sheath portion 120 is not inserted into a blood vessel of a patient during a medical procedure; however, this is not required.

The expandable sheath portion 110 is generally partially or fully formed of a flexible material (e.g., one or more flexible polymers, etc.). Generally, 60-100% (and all values and ranges therebetween) the longitudinal length of the expandable sheath portion 110 is flexible along a portion (e.g., at least 30%) or all of the longitudinal length of the expandable sheath portion 110. Generally, a portion or all of the expandable sheath portion 110 is inserted into a blood vessel of a patient during a medical procedure; however, this is not required. FIG. 3A illustrates the expandable sheath portion 110 in the unexpanded orientation and FIG. 3B illustrated the expandable sheath portion 110 in the expanded orientation.

The proximal sheath portion 120 includes a lumen that exists along 80-100% (and all values and ranges therebetween) the longitudinal length of the proximal sheath portion 120. Likewise, the expandable sheath portion 110 includes a lumen that exists along 80-100% (and all values and ranges therebetween) the longitudinal length of the expandable sheath portion 110. In one non-limiting embodiment, the cross-sectional area or diameter of the outer surface of the expandable sheath portion 110 and/or the cross-sectional area or diameter of the lumen of the expandable sheath portion 110 when in the unexpanded orientation is constant along 80-100 (and all values and ranges therebetween) the longitudinal length of the expandable sheath portion 110. In another and/or alternative non-limiting embodiment, the cross-sectional area or diameter of the outer surface of the expandable sheath portion 110 and/or the cross-sectional area or diameter of the lumen of the expandable sheath portion 110 when in the expanded orientation is constant along 80-100 (and all values and ranges therebetween) the longitudinal length of the expandable sheath portion 110. The proximal region 112 of the expandable sheath portion 110 can optionally have an increased cross-sectional area or diameter of the lumen at or near the distal end of the proximal sheath portion 120 when the expandable sheath portion is in the unexpanded orientation due to the connection of the proximal end or proximal end region of the expandable sheath portion 110 to the distal end or distal end region of the proximal sheath portion 120.

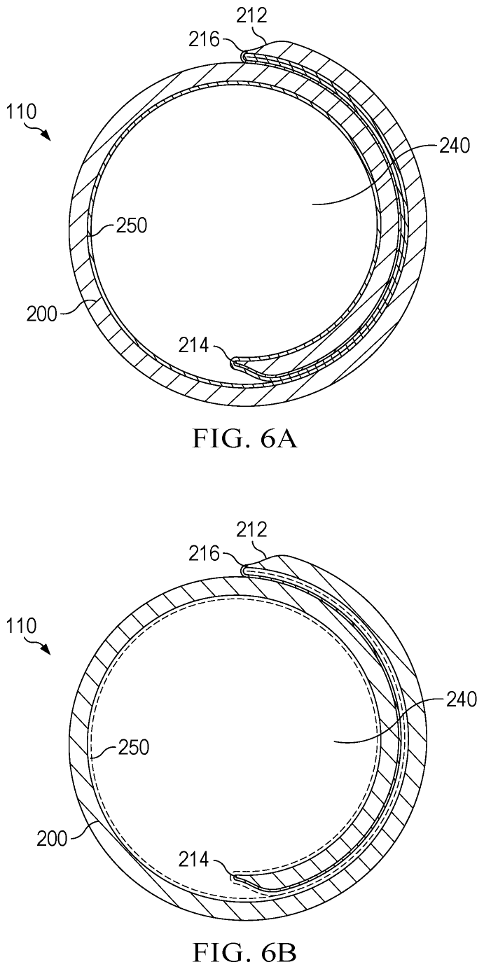

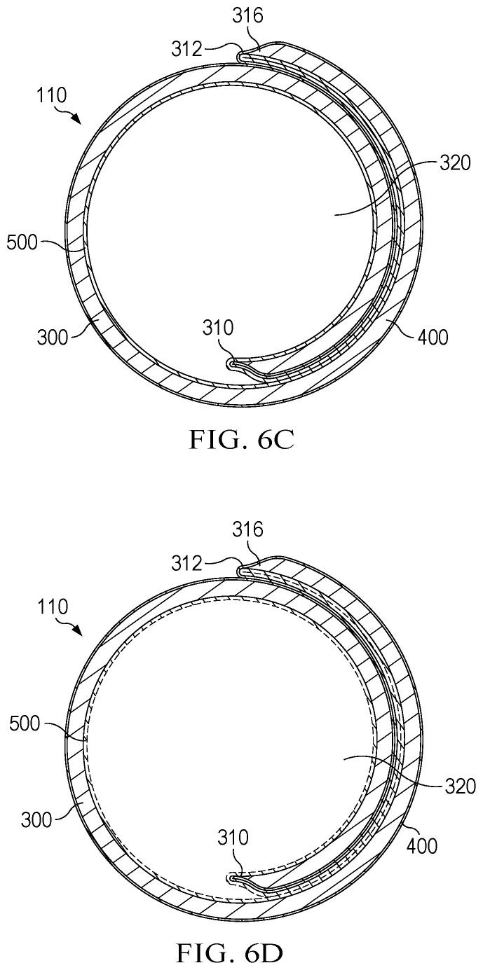

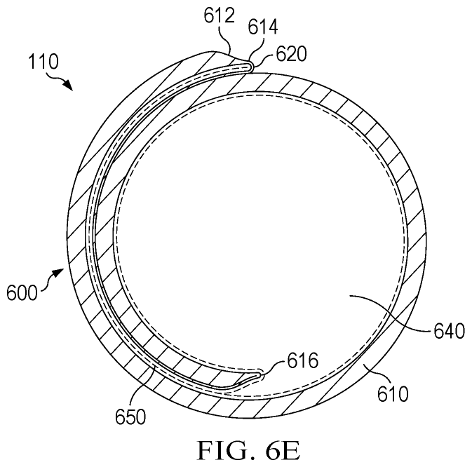

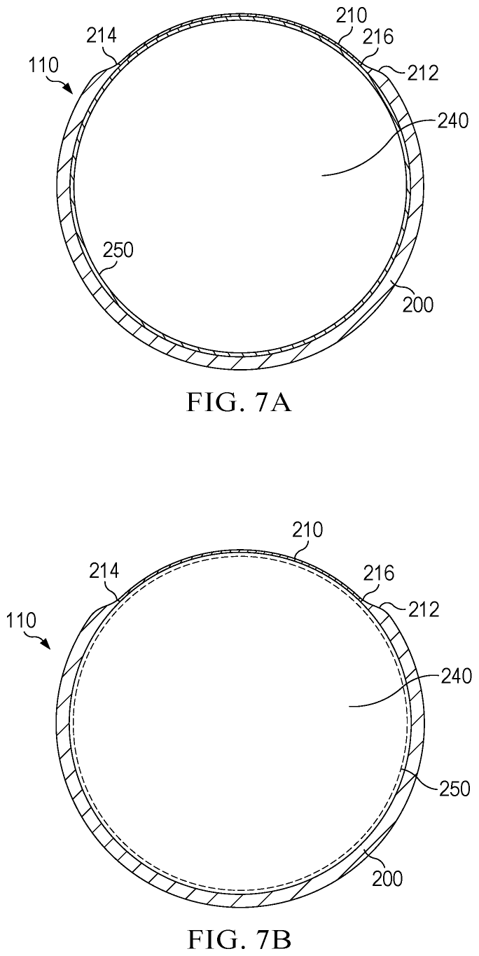

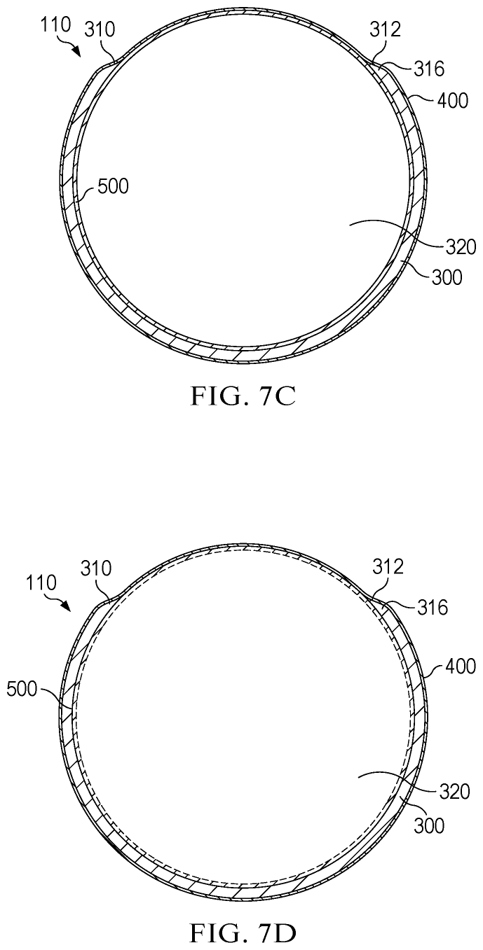

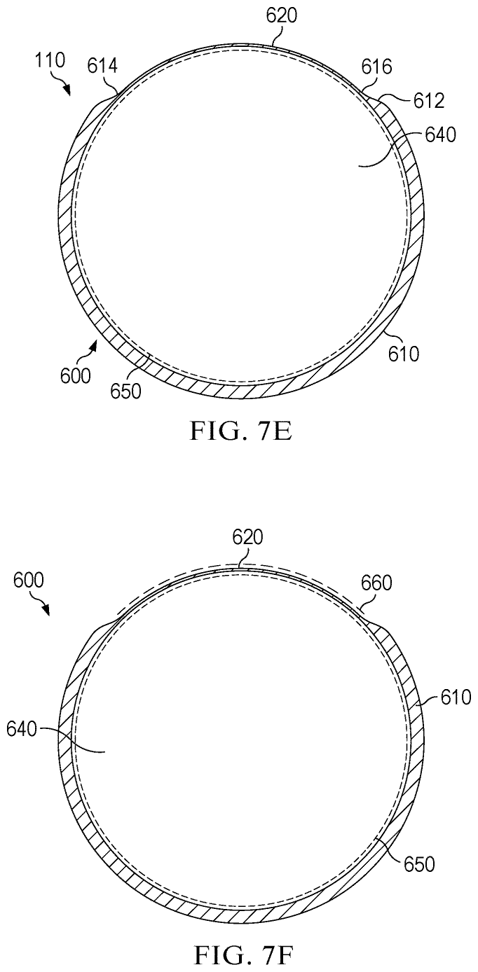

Referring now to FIGS. 6A-6E and 7A-7F, there are illustrated various cross-sections of the expandable sheath portion 110. FIGS. 6A-6E illustrate a various non-limiting cross-sections of the expandable sheath portion 110 in the fully unexpanded orientation. FIGS. 7A-7F illustrate a various non-limiting cross-sections of the expandable sheath portion 110 in the fully open or expanded orientation.

Referring now to FIGS. 6A, 6B, 7A and 7B, there is illustrated cross-section of the expandable sheath portion 110 in the fully unexpanded orientation and the fully open or fully expanded orientation that is form of first polymer portion 200 that has a first thickness and a second polymer portion 210 that has a second thickness, and wherein the average first thickness of the first polymer portion 200 is thicker than the average second thickness of the second polymer portion 210. The first and second polymer portions 200, 210 can be formed of various polymers. Non-limiting polymers include polyether-block-amide (PEBA) (e.g., Pebax™, etc.), polyvinyl acetate or polyvinyl acetate or poly(ethenyl ethanoate) (PVA or PVAC), etc. The polymer used to form the first and second polymer portions 200, 210 can be the same or different. The average first thickness of the first polymer portion 200 is generally 0.0055-0.025 inches, and the average second thickness of the second polymer portion 210 is generally 0.001-0.005 inches. In one non-limiting embodiment, the thickness of the second polymer portion 210 and the polymer used to form the second polymer portion 210 enables the second thick portion to be bendable and foldable along 5-100% (and all values and ranges therebetween) of the longitudinal length of the second polymer portion 210 when the expandable sheath is in the unexpanded orientation as illustrated in FIGS. 6A and 6B. The thickness and durometer of the first polymer portion 200 and the polymer used to form the first polymer portion 200 results in the first polymer portion 200 being flexible, but not foldable, so that the first polymer portion 200 can flex between its expanded and unshaped shape when the expandable sheath portion 110 expands and contracts between the expanded and unexpanded orientations as illustrated in FIGS. 6A, 6B, 7A and 7B.

The lumen 240 or cavity or passageway through the expandable sheath portion 110 extends along 80-100% (and all values and ranges therebetween) the longitudinal axis of the expandable sheath portion 110. The shape of the lumen 240 can be generally circular or oval when the expandable sheath 110 is in the fully expanded orientation as illustrated in FIGS. 7A and 7B; however, other shapes can be used. In one non-limiting configuration, the average diameter of the lumen 240 when the expandable sheath portion 110 is in the fully expanded position or orientation is at least 4 mm (e.g., 4-15 mm and all values and ranges therebetween).

The first and second polymer portions 200, 210 can be connected by one or more connection mechanisms (e.g., heat bonding, adhesive, mechanical connection [e.g., snap connection, clip connection, staple connection, pin connection, stitching, friction fit connection, etc.), melt bonding, welded bond, use of bonding layer or bonding strips, etc.) at connection locations 214, 216. As can be appreciated, two or more bonding arrangements can be used (e.g., adhesive and heat bonding, heat bonding and use of bonding layer such as an inner and/or outer polymer coating, etc.). As illustrated in FIGS. FIGS. 6A, 6B, 7A and 7B the end of the second polymer portion 210 is connected to the ends of the first polymer portion 200; however, it can be appreciated that the portions of the second polymer portion 210 can be also or alternatively connected to a top or bottom portion of the first polymer portions at and/or near the ends of the first polymer portion. When the second polymer portion 210 is connected to a portion of top or bottom portion of the first polymer portion, the second polymer portion does fully cover or overlie the first polymer portion about the circumference of the of the first polymer portion. In one non-limiting configuration, the second polymer portion 210 to cover or overlie 0-60% (and all values and ranges therebetween) of the first polymer portion 200 (i.e., 0% when the ends of the second polymer portion 210 are both connected to the ends of the first polymer portion 200).

As illustrated in FIGS. 6A, 6B, 7A and 7B, an optional sloped or tapered transition 212 in the first polymer portion 200 can be formed to create a thickness transition from the first polymer portion 200 to the second polymer portion 210. The sloped or tapered transition 212 is illustrated as a continuous sloped region; however, other shapes can be formed (e.g., curved or wavy, or stepped, etc.). As can be appreciated, the first polymer portion 200 and/or second polymer portion 210 can be absent a sloped transition region. The length of each of the sloped or tapered transition 212, when used, is about 0.01-15% (and all values and ranges therebetween) of the circumferential length of the first polymer portion 200. As best illustrated in FIGS. 7A and 7B, the sloped or tapered transition 212 is illustrated as sloping downwardly from the top edge of the maximum thick portion of the first polymer portion 200. In such an arrangement, the inner surface of the first and second polymer portions 200, 210 when the expand sheath portion is in the fully expanded orientation forms a circular lumen having a constant radius from the inner surface about 80-100% (and all values and ranges therebetween) of the perimeter of the inner surface as illustrated in FIGS. 7A and 7B. As can be appreciated, the sloped or tapered transition 212 can alternatively be configured to slope upwardly from the bottom edge of the maximum bottom edge of the first polymer portion 200. In such an arrangement, the outer surface of the first and second polymer portions 200, 210 when the expand sheath portion is in the fully expanded orientation forms a circular lumen having a constant radius from the outer surface about 80-100% (and all values and ranges therebetween) of the perimeter of the outer surface.

The second polymer portion 210 has a length of 5-40% (and all values and ranges therebetween) of the circumferential length (i.e., circumference) of the outer surface of the first polymer portion 200 as measured between a) the ends 214, 216 of the optional sloped or tapered transition 212 of the first polymer portion 200 or b) the ends of the first polymer portion 200 when there is no optional sloped or tapered transition 212, and/or 2-45% (and all values and ranges therebetween) of the circumferential length of the first polymer portion 200 when measured about the outer circumference of the first polymer portion 200 when the expandable sheath portion 110 is in the fully expanded position or orientation. The length of the outer perimeter of the second polymer portion 210 is generally less than the length of the outer perimeter of first polymer portion 200 when measured about the outer circumference of the first polymer portion 200 when the expandable sheath portion 110 is in the fully expanded position or orientation. Generally the length of the outer perimeter of the second polymer portion 210 is 10-45% (and all values and ranges therebetween) the length of the outer perimeter of first polymer portion 200 when measured about the outer circumference of the first polymer portion 200 when the expandable sheath portion 110 is in the fully expanded position or orientation.

An optional liner 250 (e.g., PTFE liner, etc.) can be positioned in the interior of the lumen 240 of the expandable sheath portion 110. FIGS. 6A and 7A illustrate the use of the optional liner 250, and FIGS. 6B and 7B illustrate the optional liner 250 as a dashed line to represent that the optional liner may or may not be used in the expandable sheath portion 110. When the optional liner 250 is used, the optional liner 250 can be positioned in and/or connected to the inner surface first and/or second polymer portions 200, 210. The optional liner 250 can be a tubular shaped liner that has a wall thickness of at least 0.001 inches (e.g., 0.001-0.01 inches and all values and ranges therebetween) and has a circular or oval cross-sectional shape; however, other shapes can be used. Generally, the shape of the tubular liner 250 is the same as the shape of the lumen 240 that is formed by the interior surface of the first and second polymer portions 200, 210 when the expandable sheath portion is in the expanded orientation, and the outer cross-sectional size of the optional liner 250 is generally ±10 (and all values and ranges therebetween) of the cross-sectional size of the lumen 240 or interior surface of the first and second polymer portions 200, 210 when the expandable sheath portion 110 is in the expanded orientation.

The outer surface of the optional liner 250 can optionally include surface etching and/or roughening (not shown) to facilitate in the bonding of the optional liner 250 to the inner surface of the first and/or second polymer portions 200, 210. The optional liner 250 generally has a longitudinal length of 50-100% (and all values and ranges therebetween) the longitudinal length of the lumen 240 formed by the inner surface of the first and second polymer portions 200, 210. The bonding of the optional liner to the first and/or second polymer portions 200, 210 can be by a heat process (e.g., heat flow process, thermocompression process, etc.) and/or by use of an adhesive, and/or other connecting process or arrangement.

In one non-limiting method for forming the expandable sheath portion 110, the first polymer portion 200 is positioned in the fully unexpanded position or orientation. Generally, the first polymer portion 200 is initially or first formed in the fully expanded orientation as illustrated in FIGS. 7A and 7B. The formation of the first polymer portion 200 in the fully expanded orientation can be by extrusion, molding, 3D printing, cutting, stamping, etc. In one non-limiting method, the first polymer portion 200 is extruded in the shape of its fully expanded orientation. The first polymer portion 200 can be extruded so as to have spaced ends 214, 216 after the extrusion process. Alternatively, the first polymer portion 200 can be extruded or molded as a tube, and thereafter a portion of the tube is cut out along the longitudinal length of the extruded tube to form the spaced apart ends 214, 216. As can be appreciated, other methods can be used to form the first polymer portion 200.

The second polymer portion 210 can be also be formed by a variety of processes (e.g., molding, extrusion, stamping, cutting, 3D printing, etc.). After the second polymer portion 210 is formed, one end of the second polymer portion 210 is connected to one end of the first polymer portion 200 and the other end of the second polymer portion 210 is connected to the other end of the first polymer portion 200. The type of connection used is non-limiting (e.g., adhesive, melted or heat connection, mechanical connection, etc.).

After the first and second polymer portions 200, 210 are formed and connected together, the first and second polymer portions 200, 210, and the optional liner 250 are typically bent about and/or folded about a mandrel, not shown, to form the expandable portion of the expandable sheath 110 in the fully unexpanded position or orientation as illustrated in FIGS. 6A and 6B. As can be appreciated, the first polymer portion 200 could be initially formed in the fully unexpanded position or orientation prior to connecting the second polymer portion 210 to the first polymer portion 200.

When the first polymer portion 200 is in the fully unexpanded position or orientation as illustrated in FIGS. 6A and 6B, the ends 214, 216 and end regions of the first polymer portion 200 partially or fully overlap one another, and a portion or all of the second polymer portion 210 (e.g., 60-100% of the longitudinal length of the second polymer portion 210 is positioned between the overlapped ends and end regions of the first polymer portion 200. When the first polymer portion 200 is in the fully unexpanded position or orientation, 0-50% (and all values and ranges therebetween) of the longitudinal length of the second polymer portion 210 is not positioned between the overlapped ends and end regions of the first polymer portion 200. When the expandable sheath portion 100 is in the fully unexpanded position or orientation, the average diameter of the lumen of the expandable sheath portion 110 of the expandable sheath 100 is at least 10% less (e.g., 10-400% less and all values and ranges therebetween) than the average diameter of the lumen of the expandable sheath portion 110 in the fully expanded position or orientation.

As illustrated in FIGS. 6A and 6B, the longitudinal length (e.g., width) of the second polymer portion 210 can be selected to limit the amount of overlap of the two ends and end region of the first polymer portion 200. In another and/or alternative non-limiting configuration, no more than 30% (e.g., 0-30% and all values and ranges therebetween) of the longitudinal length of the second polymer portion 210, that is not located between overlapping ends and end regions of the first polymer portion 200, is located in the lumen of the expandable sheath portion 110 when the expandable sheath portion 110 is in the fully unexpanded position or orientation. As illustrated in FIGS. 6A and 6B, less than 5% of the longitudinal length of the second polymer portion 210, that is not located between overlapping ends and end regions of the first polymer portion 200, is located in the lumen of the expandable sheath portion 110 when the expandable sheath portion 110 is in the fully unexpanded position or orientation.

When the expandable sheath portion 110 includes optional liner 250, a portion of the optional liner 250 is caused to be positioned between the overlapped ends and end regions of the first polymer portion 200 when the expandable sheath portion 110 is in the fully unexpanded position or orientation as illustrated in FIGS. 6A and 6B.

An optional non-stick layer (not shown) (e.g., PTFE polymer portion, etc.) can be applied to one or both sides of all or a portion of the second polymer portion 210, and/or on one or both sides of all or a portion of the first polymer portion 200. An example of the use of the optional non-stick layer is discussed below with reference to FIG. 7F. The optional non-stick layer, when used, is positioned on a portion of the outer surface of the second polymer portion 210 that is located between the ends of the first polymer portion 200. As can be appreciated, optional non-stick layer can be applied to a) 5-100% (and all values and ranges therebetween) of the outer surface and/or the inner surface of the first polymer portion and/or b) 5-100% (and all values and ranges therebetween) of the outer surface and/or the inner surface of the second polymer portion. When the optional liner 250 is not used, the optional non-stick layer can be positioned on a portion of the inner surface of the first and/or second polymer portions. When the optional liner 250 is used, the optional non-stick layer typically is not positioned on a portion of the inner surface of the first and/or second polymer portions. The location of the optional non-stick layer on either or both the first polymer portion 200 and the second polymer portion 210 is selected to inhibit or prevent the first polymer portion and the second polymer portion from sticking together during the forming and annealing of the expandable sheath portion 110 in the fully unexpanded position or orientation. Generally, the melting point of the optional non-stick layer is greater than the melting point of the first and second polymer portions 200, 210. Generally the material used to form the optional non-stick layer is different from the material used to form the first and second polymer portions. The average thickness of the optional non-stick layer is typically less than the average thickness of the first polymer portion 200. After the forming and annealing of the expandable sheath portion 110 in the fully unexpanded position or orientation, the optional non-stick layer is removed from the first and/or second polymer portions.

Referring now to FIGS. 6C, 6D, 7C and 7D, the expandable sheath portion 110 of the expandable sheath 100 is formed of first and second polymer portions 300, 400. The first polymer portion 300 is a non-continuous layer that has two ends 310, 312 that are not connected together. The first polymer portion 300 is optionally configured such that a) the two ends 310, 312 or two end regions overlap one another when the first polymer portion 300 of the expandable sheath 100 is in the fully unexpanded orientation as illustrated in FIGS. 6C and 6D, and b) the two ends 310, 312 or two end regions are separated from one another when the first polymer portion 300 of the expandable sheath 100 is in the fully expanded orientation.

The second polymer portion 400 is a continuous layer that forms an outer layer of the expandable sheath portion 110 of the expandable sheath 100. The second polymer portion 400 is optionally a non-stretchable material, and wherein the second polymer portion 400 optionally has a constant thicknesses, and wherein the second polymer portion 400 is formed of a same or different material from the first polymer portion 300, and wherein a durometer and/or thickness of the first polymer portion 300 is optionally greater than a durometer and/or thickness of the second polymer portion 400.

The thickness and durometer of the first polymer portion 300 and the polymer used to form the first polymer portion 300 results in the first polymer portion 300 being flexible, but not foldable, so that the first polymer portion 300 can flex between its expanded and unshaped shape when the expandable sheath portion 110 expands and contracts between the expanded and unexpanded orientations as illustrated in FIGS. 6C, 6D, 7C and 7B. The thickness and durometer of the second polymer portion 400 and the polymer used to form the second polymer portion 400 results in the second polymer portion 400 being flexible and foldable so that the second polymer portion 400 can bend and fold between its expanded and unshaped shape in the region between ends 310, 312 of the first polymer portion 300 when the expandable sheath portion 110 expands and contracts between the expanded and unexpanded orientations as illustrated in FIGS. 6C, 6D, 7C and 7B.

The first polymer portion 300 can be formed in a tube or be formed in a tube that includes the two spaced apart ends 310, 312. The polymer used to form the first polymer portion 300 can include one or more of polyether-block-amide (PEBA) (e.g., Pebax™, etc.), polyvinyl acetate or polyvinyl acetate or poly(ethenyl ethanoate) (PVA or PVAC); however, other polymers can be used. The wall thickness of the tube formed by the first polymer portion 300 is generally 0.0055-0.025 inches (and all values and ranges therebetween). When a full tube is formed, a portion of the tube is cut out or otherwise removed to form the two spaced apart ends 310, 312. As can be appreciated, a semi-tube can be initially formed with the two spaced apart ends 310, 312. The tube can be formed by a variety of processes (e.g., extrusion, molding, 3D printing, cutting, stamping, etc.).

The tube or semi-tube formed by the first polymer portion 300 includes a lumen 320 or cavity or passageway that extends along 80-100% (and all values and ranges therebetween) the longitudinal axis of the tube. The shape of the lumen 320 can be generally circular or oval; however, other shapes can be used. In one non-limiting configuration, the average diameter of the lumen 320 when the expandable sheath portion 110 is in the fully expanded position or orientation is at least 4 mm (e.g., 4-15 mm and all values and ranges therebetween).

The end regions of the first polymer portion 300 can optionally include a tapered region 316. The taper region can be a continuous sloped region; however, other shapes can be formed (e.g., curved or wavy, or stepped, etc.). The length of each of the tapered regions 316 of the ends 310, 312, when used, is about 0.01-15% (and all values and ranges therebetween) of the circumferential length of the tube. As can be appreciated, the first polymer portion 300 can be absent a taper or sloped transition region.

The gap formed between ends 310, 312 run along 50-100% (and all values and ranges therebetween) of a longitudinal length of the tube. The width of the gap is about 2-45% (and all values and ranges therebetween) of the outer perimeter of the expandable sheath portion 110 when in the fully open or expanded orientation. The width of the gap can be uniform along 50-100% (and all values and ranges therebetween) of a longitudinal length of the first polymer portion.

The second polymer portion 400 is positioned completely about the outer perimeter of the first polymer portion 300 along 10-100% (and all values and ranges therebetween) along the longitudinal length of the expandable sheath portion 110, and also spans the gap between ends 310, 312 when the first polymer portion in the fully expanded orientation as illustrated in FIGS. 7C and 7D. The second polymer portion 400 is illustrated in FIGS. 7C and 7D has forming the outer layer of the expandable sheath portion 110. The portion of the second polymer portion 400 that spans the gap between ends 310, 312 is the spanning portion of the second polymer portion 400. The second polymer portion 400 extends 10-100% (and all values and range therebetween) of the longitudinal length of the expandable sheath portion 110. When ends 310, 312 optionally includes a tapered region 316, the second polymer portion 400 can be connected to the outer surface of the tapered region 316, and thereby also slope along the tapered region 316 as illustrated in FIGS. 6C, 6D, 7C and 7D.

The polymer used to form the second polymer portion 400 can include polyether-block-amide (PEBA) (e.g., Pebax™, etc.), polyvinyl acetate or polyvinyl acetate or poly(ethenyl ethanoate) (PVA or PVAC). The material of the first and second polymer portions 300, 400 can be the same or different. Generally, the durometer of the polymer of the first polymer portion 300 is greater than the durometer of the polymer of the second polymer portion 400; however, this is not required. The average thickness of the second polymer portion 400 is generally less than the average thickness of the first polymer portion 300. In one non-limiting embodiment, the thickness of the second polymer portion 400 is 0.001-0.005 inches (and all values and ranges therebetween).

An optional liner 500 (e.g., PTFE liner, etc.) can be positioned in the interior of the lumen 320 of the expandable sheath portion 110. FIGS. 6C and 7C illustrate the expandable sheath portion 110 as including liner 500, and FIGS. 6D and 7D illustrate the liner 500 in a dashed line to indicate that the liner 500 is optional. When the optional liner 500 is used, the optional liner 500 can be positioned in and/or optionally connected to a) a portion or all of the inner surface second polymer portion that spans the gap between ends 310, 312 of the first polymer portion 300 and/or b) a portion or all of the insert surface of the first polymer portion 300. The optional liner 500 can be a tubular shaped liner that has a wall thickness of at least 0.001 inches (e.g., 0.001-0.01 inches and all values and ranges therebetween) and has a circular or oval cross-sectional shape in the fully open orientation as illustrated in FIGS. 7C and 7D. Generally, the shape of the tubular liner 500 is the same as the shape of the lumen that is formed by the first polymer portion 300 and the inner surface of the second polymer portion 400 that spans the gap between ends 310, 312 of the first polymer portion 300 when the expandable sheath portion 110 is in the fully unexpanded orientation as illustrated in FIGS. 7C and 7D. The outer cross-sectional size of the optional liner 500 is generally ±10 (and all values and ranges therebetween) of the cross-sectional size of the lumen that is formed by the first polymer portion 300 and the inner surface of the second polymer portion 400 that spans the gap between ends 310, 312 of the first polymer portion 300 when the expandable sheath portion 110 is in the fully unexpanded orientation as illustrated in FIGS. 7C and 7D.

The outer surface of the optional liner 500 can optionally include surface etching and/or roughening to facilitate in the bonding of the optional liner 500 to a) the inner surface of the lumen that is formed by the inner surface first polymer portion 300, and/or b) the inner surface of the lumen that is formed by the second polymer portion that spans the gap between ends 310, 312 of the first polymer portion 300. The optional liner 500 generally has a longitudinal length of 50-100% (and all values and ranges therebetween) the longitudinal length of the lumen 320. The bonding of the optional liner 500 to one or more portions of the lumen can be by a heat process (e.g., heat flow process, thermocompression process, etc.) and/or by use of an adhesive, and or by some other connection process.

During the formation of the expandable sheath portion 110, the first polymer portion 300 is bent into the fully unexpanded position or orientation. Generally, the first polymer portion 300 is first formed in the fully expanded orientation. When the first polymer portion 300 is first formed in the fully expanded orientation, the second polymer portion 400 is inserted about the outer surface of the first polymer portion 300. The first polymer portion 300 can optionally be bent partially or fully to the fully unexpanded orientation to facilitate in the insertion of the second polymer portion 400 about the outer surface of the first polymer portions 300. After the second polymer portion 400 is portioned about the outer surface of the first polymer portions 300, the second polymer portion 400 can optionally be connected to the outer surface of the first polymer portion by one or more arrangements (e.g., adhesive connection, heat connection, melted or reflow connection, mechanical connection, etc.). The optional liner 500, when used, can be inserted into the lumen of the expandable sheath portion 110 prior to, during or after the second polymer portion 400 is positioned about the outer surface of the first polymer portions 300. After the optional liner 500, when used, is inserted into the lumen of the expandable sheath portion 110, the optional liner 500 can optionally be connected to the inner surface of the first and/or second polymer portions 300, 400 one or more arrangements (e.g., adhesive connection, heat connection, melted or reflow connection, mechanical connection, etc.).

After the second polymer portion 400 is positioned about the outer surface of the first polymer portions 300, and the optional liner 500 is inserted into the lumen, the first polymer portion 300 and second polymer portion 400, and the optional liner 500 are typically bent about and/or folded about a tapered mandrel, not shown, to form the expandable portion of the expandable sheath in the fully unexpanded position or orientation. As can be appreciated, the first polymer portion 300 and second polymer portion 400 could be initially formed in the fully unexpanded position or orientation.

When the first polymer portion 300 is in the fully unexpanded position or orientation, the ends 310, 312 and end regions of the first polymer portion 300 partially or fully overlap one another, and a portion or all of the spanning portion second polymer portion 400 (e.g., 60-100% of the spanning portion) is positioned between the overlapped end regions of the first polymer portion 300 as illustrated in FIGS. 6C and 6D. When the first polymer portion 300 is in the fully unexpanded position or orientation, 0-50% (and all values and ranges therebetween) of the longitudinal length of the spanning portion of the second polymer portion 400 that is not positioned between the overlapped end regions of the first polymer portion 300 is located in the lumen of the expandable sheath portion 110. In the fully unexpanded position or orientation, the average diameter of the lumen of the expandable sheath portion 110 is at least 10% less (e.g., 10-400% less and all values and ranges therebetween) than the average diameter of the lumen of the expandable sheath portion 110 in the fully expanded position or orientation.

As illustrated in FIGS. 6C and 6D, the longitudinal length of the spanning portion of the second polymer portion 400 can be selected to limit the amount of overlap of the two ends 310, 312 and end regions of the first polymer portion 300.

When the expandable sheath portion 110 includes optional liner 500, a portion of the optional liner 500 is caused to be positioned between the overlapped ends and end regions of the first polymer portion 300 when the expandable sheath portion 110 is in the fully unexpanded position or orientation as illustrated in FIGS. 6C and 6D.

An optional non-stick layer (e.g., PTFE polymer portion, etc.), not shown, can be applied to one or both sides of all or a portion of the spanning portion of the second polymer portion 400, and/or on one or both sides of all or a portion of the first polymer portion 300. An example of the use of the optional non-stick layer is discussed below with reference to FIG. 7F.

The optional non-stick layer, when used, is positioned on a portion of the outer surface of the second polymer portion 400 that is located between the ends of the first polymer portion 300. As can be appreciated, optional non-stick layer can be applied to a) 5-100% (and all values and ranges therebetween) of the outer surface and/or the inner surface of the first polymer portion and/or b) 5-100% (and all values and ranges therebetween) of the outer surface and/or the inner surface of the second polymer portion. When the optional liner 500 is not used, the optional non-stick layer can be positioned on a portion of the inner surface of the second polymer portion 400.

When the optional liner 500 is used, the optional non-stick layer typically is not positioned on a portion of the inner surface of the first and/or second polymer portions. The location of the optional non-stick layer on either or both the first polymer portion 300 and the second polymer portion 400 is selected to inhibit or prevent the spanning portion of the second polymer portion 400 and the first polymer portion 300 from sticking together during the forming and annealing of the expandable sheath portion 110 in the fully unexpanded position or orientation. Generally, the melting point of the optional non-stick layer is greater than the melting point of the first and second polymer portions 300, 400. Generally the material used to form the optional non-stick layer is different from the material used to form the first and second polymer portions. The average thickness of the optional non-stick layer is typically less than the average thickness of the first polymer portion 300. After the forming and annealing of the expandable sheath portion 110 in the fully unexpanded position or orientation, the optional non-stick layer is removed from the first and/or second polymer portions.

Referring now to FIGS. 6E and 7E, there is illustrated an expandable sheath portion 110 that is formed of a continuous first polymer portion 600 that has a first thick portion 610 and a second thick portion 620, wherein the first thick portion 610 is thicker than the thickness of the second thick portion 620. The first polymer portion can be formed of various polymers. Non-limiting polymers include polyether-block-amide (PEBA) (e.g., Pebax™, etc.), polyvinyl acetate or polyvinyl acetate or poly(ethenyl ethanoate) (PVA or PVAC), etc. The thickness of the first thick portion 610 is generally 0.0055-0.025 inches, and the thickness of the second thick portion 620 is 0.001-0.005 inches. The average thickness of the first thick portion is greater than the average thickness of the second thick portion. In one non-limiting embodiment, the thickness of the second thick portion 620 and the polymer used to form the second thick portion 620 enables the second thick portion to be bendable and foldable along 5-100% (and all values and ranges therebetween) of the longitudinal length of the second thick portion 620 when the expandable sheath is in the unexpanded orientation as illustrated in FIG. 6E. The thickness and durometer of the first thick portion 610 and the polymer used to form the first thick portion 610 results in the first thick portion being flexible, but not foldable, so that the first thick portion can flex between its expanded and unshaped shape when the expandable sheath portion 110 expands and contracts between the expanded and unexpanded orientations as illustrated in FIGS. 6E and 7E. The thickness and durometer of the second thick portion 620 and the polymer used to form the second thick portion 620 results in the second thick portion being flexible and foldable, so that the second thick portion can bend and fold between its expanded and unshaped shape when the expandable sheath portion 110 expands and contracts between the expanded and unexpanded orientations as illustrated in FIGS. 6E and 7E.

The lumen 640 or cavity or passageway through the expandable sheath portion 110 extends along 80-100% (and all values and ranges therebetween) the longitudinal axis of the expandable sheath portion 110. The shape of the lumen 640 can be generally circular or oval when the expandable sheath 110 is in the fully expanded orientation as illustrated in FIG. 7E; however, other shapes can be used. In one non-limiting configuration, the average diameter of the lumen 640 when the expandable sheath portion 110 is in the fully expanded position or orientation is at least 4 mm (e.g., 4-15 mm and all values and ranges therebetween).

As illustrated in FIGS. 6E and 7E, an optional sloped or tapered transition 612 in the first polymer portion 600 can be formed to create a thickness transition from the first thick portion 610 to the second thick portion 620. As can be appreciated, the first polymer portion 600 can be absent a taper or sloped transition region. The sloped or tapered transition 612 is illustrated as a continuous sloped region; however, other shapes can be formed (e.g., curved or wavy, or stepped, etc.). The length of each of the sloped or tapered transition 612, when used, is about 0.01-15% (and all values and ranges therebetween) of the circumferential length of the first thick portion 610. As best illustrated in FIG. 7E, the sloped or tapered transition 612 is illustrated as sloping downwardly from the top edge of the maximum thick portion of the first thick portion 610. In such an arrangement, the inner surface of the first and second thick portions 610, 620 when the expand sheath portion is in the fully expanded orientation forms a circular lumen having a constant radius from the inner surface about 80-100% (and all values and ranges therebetween) of the perimeter of the inner surface as illustrated in FIG. 7E. As can be appreciated, the sloped or tapered transition 612 can alternatively be configured to slope upwardly from the bottom edge of the maximum thick portion of the first thick portion 610. In such an arrangement, the outer surface of the first and second thick portions 610, 620 when the expand sheath portion is in the fully expanded orientation forms a circular lumen having a constant radius from the outer surface about 80-100% (and all values and ranges therebetween) of the perimeter of the outer surface.

The second thick portion 620 has a length of 5-40% (and all values and ranges therebetween) of the circumferential length of the outer surface of the first polymer portion 600 as measured between a) the ends 614, 616 of the optional sloped or tapered transition 612 of the first thick portion 610 or b) the ends of the first thick portion 610 when there is no optional sloped or tapered transition 612, and/or 2-45% (and all values and ranges therebetween) of the circumferential length of the first thick portion 610 when measured about the outer circumference of the first polymer portion 600 when the expandable sheath portion 110 is in the fully expanded position or orientation. The length of the outer perimeter of the second thick portion 620 is generally less than the length of the outer perimeter of first thick portion 610 when measured about the outer circumference of the first polymer portion 600 when the expandable sheath portion 110 is in the fully expanded position or orientation. Generally the length of the outer perimeter of the second thick portion 620 is 10-45% (and all values and ranges therebetween) the length of the outer perimeter of first thick portion 610 when measured about the outer circumference of the first polymer portion 600 when the expandable sheath portion 110 is in the fully expanded position or orientation.

An optional liner 650 (e.g., PTFE liner, etc.) can be positioned in the interior of the lumen 640 of the expandable sheath portion 110. FIGS. 6E and 7E illustrate the liner 650 in a dashed line to indicate that the liner 650 is optional. When the optional liner 650 is used, the optional liner 650 can be positioned in and/or connected to the inner surface first polymer portion 600. The optional liner 650 can be a tubular shaped liner that has a wall thickness of at least 0.001 inches (e.g., 0.001-0.01 inches and all values and ranges therebetween) and has a circular or oval cross-sectional shape; however, other shapes can be used. Generally, the shape of the tubular liner 650 is the same as the shape of the lumen 640 that is formed by the interior surface of the first polymer portion 600 when the expandable sheath portion is in the expanded orientation, and the outer cross-sectional size of the optional liner 650 is generally ±10 (and all values and ranges therebetween) of the cross-sectional size of the lumen 640 or interior surface of the first polymer portion 200 when the expandable sheath portion 110 is in the expanded orientation.

The outer surface of the optional liner 650 can optionally include surface etching and/or roughening to facilitate in the bonding of the optional liner 650 to the inner surface of the first polymer portion 600. The optional liner 650 generally has a longitudinal length of 50-100% (and all values and ranges therebetween) the longitudinal length of the lumen 640 formed by the inner surface of the first polymer portion 600. The bonding of the optional liner to the first polymer portion can be by a heat process (e.g., heat flow process, thermocompression process, etc.) and/or by use of an adhesive, and/or other connecting process or arrangement.

In one non-limiting method for forming the expandable sheath portion 110, the first polymer portion 600 is positioned in the fully unexpanded position or orientation. Generally, the first polymer portion is initially or first formed in the fully expanded orientation as illustrated in FIG. 7E. The formation of the first polymer portion 600 in the fully expanded orientation can be by extrusion, molding, 3D printing, cutting, stamping, etc. In one non-limiting method, the first polymer portion is extruded in the shape of its fully expanded orientation. After the first polymer portion 600 is first formed in the fully expanded orientation, the first polymer portion 600, and the optional liner 650 are typically bent about and/or folded about a mandrel, not shown, to form the expandable portion of the expandable sheath 110 in the fully unexpanded position or orientation as illustrated in FIG. 6E. As can be appreciated, the first polymer portion 600 could be initially formed in the fully unexpanded position or orientation.

When the first polymer portion 600 is in the fully unexpanded position or orientation as illustrated in FIG. 6E, the end regions of the first thick portion 610 partially or fully overlap one another, and a portion or all of the second thick portion 620 (e.g., 60-100% of the longitudinal length of the second thick portion 620 is positioned between the overlapped end regions of the first thick portion 610. When the first polymer portion 600 is in the fully unexpanded position or orientation, 0-50% (and all values and ranges therebetween) of the longitudinal length of the second thick portion 620 is not positioned between the overlapped end regions of the first thick portion 610. When the expandable sheath portion 100 is in the fully unexpanded position or orientation, the average diameter of the lumen of the expandable sheath portion 110 of the expandable sheath 100 is at least 10% less (e.g., 10-400% less and all values and ranges therebetween) than the average diameter of the lumen of the expandable sheath portion 110 in the fully expanded position or orientation.

As illustrated in FIG. 6E, the longitudinal length of the second thick portion 620 can be selected to limit the amount of overlap of the two end region of the first thick portion 610. In another and/or alternative non-limiting configuration, no more than 30% (e.g., 0-30% and all values and ranges therebetween) of the longitudinal length of the second thick portion 620, that is not located between overlapping end regions of the first thick portion 610, is located in the lumen of the expandable sheath portion 110 when the expandable sheath portion 110 is in the fully unexpanded position or orientation. As illustrated in FIG. 6E, less than 5% of the longitudinal length of the second thick portion 620, that is not located between overlapping end regions of the first thick portion 610, is located in the lumen of the expandable sheath portion 110 when the expandable sheath portion 110 is in the fully unexpanded position or orientation.

When the expandable sheath portion 110 includes optional liner 650, a portion of the optional liner 450 is caused to be positioned between the overlapped end regions of the first thick portion 610 when the expandable sheath portion 110 is in the fully unexpanded position or orientation as illustrated in FIG. 6E.

Referring now to FIG. 7F, an optional non-stick layer 660 (e.g., PTFE polymer portion, etc.) can be applied to one or both sides of all or a portion of the second thick portion 620, and/or on one or both sides of all or a portion of the first thick portion 610. As illustrated in FIG. 7F, the optional non-stick layer 660 is positioned on a portion of the outer surface of the second thick portion 620 that is located between the ends of the first thick portion 610. As can be appreciated, optional non-stick layer 660 can be applied to a) 5-100% (and all values and ranges therebetween) of the outer surface and/or the inner surface of the first thick portion and/or b) 5-100% (and all values and ranges therebetween) of the outer surface and/or the inner surface of the second thick portion. When the optional liner 650 is not used, the optional non-stick layer 660 can be positioned on a portion of the inner surface of the first and/or second thick portions. When the optional liner 650 is used, the optional non-stick layer 660 typically is not positioned on a portion of the inner surface of the first and/or second thick portions. The location of the optional non-stick layer 660 on either or both the first thick portion 610 and the second thick portion 620 is selected to inhibit or prevent the first thick portion 610 and the second thick portion 620 from sticking together during the forming and annealing of the expandable sheath portion 110 in the fully unexpanded position or orientation. Generally, the melting point of the optional non-stick layer 660 is greater than the melting point of the first polymer portion 600. Generally the material used to form the optional non-stick layer 660 is different from the material used to form the first polymer portion 600. The average thickness of the optional non-stick layer 660 is typically less than the average thickness of the first thick portion 610 of the first polymer portion 600. After the forming and annealing of the expandable sheath portion 110 in the fully unexpanded position or orientation, the optional non-stick layer 660 is removed from the first polymer portion 600.

Referring now to FIGS. 2A and 3A, the expandable sheath portion 110 can optionally include a taper end tip 130. The tapered end tip 130 is generally at least 0.1 mm in length (e.g., 0.1-10 mm and all values and ranges therebetween) or 0.001-5% (and all values and ranges therebetween) the longitudinal length of the expandable sheath portion 110. The tapered end tip 130 can optionally be formed by a heating process. A breakable seam can optionally be formed at the tapered end tip 130. The breakable seam, when used, can be formed by the melting together of the edges of the tapered end tip during the formation of the tapered end tip; however, this is not required. As can be appreciated, an adhesive can alternatively be used to maintain the tapered shape of the tapered end tip 130 until the tip is broken open by a medical device MD or some of other type of medical instrument. Other connection arrangements can also be used (e.g., hook and loop fastener, etc.).

In one non-limiting method, when the medical device MD is inserted through the expandable sheath portion 110 and past the tapered end tip 130, the seam, when used, is caused to be broken as the expandable portion expands to the unexpanded position. As can be appreciated, a tool can be inserted into the expandable portion and optionally the tip after at least a portion of the expandable portion in inserted into the patient to break connection points in the expandable portion and optionally the tip prior to inserting the medical device through the expandable sheath portion. When the expandable sheath portion 110 includes an optional liner (e.g., PTFE liner, etc.), the liner generally does not extend to the distal end of the expandable sheath portion 110 since the optional liner can interfere with the tapering of the distal end portion and the formation of the breakable seam. A recess region 134 can be formed when the expandable sheath portion 110 is expanded and the breakable seam 132 is broken.

As can be appreciated, an optional continuous outer polymer layer (not shown) can be applied to the outer surface of first polymer portion of the expandable sheath portion 110 illustrated in FIGS. 6E and 7E, or the outer surface of first and second polymer portions of the expandable sheath portion 110 illustrated in FIGS. 6A, 6B, 7A and 7B, or the outer surface of second polymer portion of the expandable sheath portion 110 illustrated in FIGS. 6C, 6D, 7C and 7D. This optional continuous outer polymer layer can be a stretchable polymer layer. When the optional continuous outer polymer layer is a stretchable polymer layer, the optional continuous outer polymer layer can be used to a) facilitate in maintaining the expandable sheath portion 110 in the unexpanded orientation until a medical device or other type of medical device is passed through the lumen of the expandable sheath portion 110 to cause the expandable sheath portion 110 to expand to the expanded orientation, and/or b) facilitate in causing one or more portions of the expandable sheath portion 110 to return to the unexpanded orientation after the medical device has passed fully or partially through the expandable sheath portion 110.

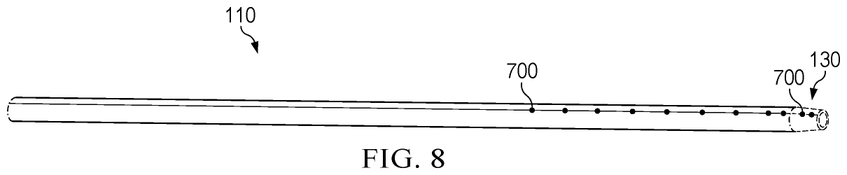

Referring now to FIGS. 2A, 3A, 4 and 8, the expandable sheath portion 110 can optionally include a taper end tip 130. The tapered end tip 130 is generally at least 0.1 mm in length (e.g., 0.1-10 mm and all values and ranges therebetween). The tapered tip can optionally be formed by a heating process. A breakable seam or one or more break points 700 can optionally be formed on the tapered end tip 130 as illustrated in FIG. 8. The breakable seam or one or more break points 700 can be formed by the melting together of the edges of the tapered end tip during the formation of the tapered end tip, and/or by use of an adhesive; however, other arrangements can be used to form the breakable seam or one or more break points 700 (e.g., adhesive, etc.). The breakable seam or one or more break points 700 can be used to facilitate in maintaining the tapered end tip in the unexpanded orientation. When the medical device is inserted through the expandable sheath portion 110 and through the tapered end tip 130 or a tool is inserted through the expandable sheath portion 110 and through the tapered end tip 130 to break the break points 700 or breakable seam prior to inserting the medical device through the expandable sheath portion, the one or more break points 700 or breakable seam can be caused to be broken, thereby allowing the expandable sheath portion and the optional tapered end tip to expand to the expanded orientation and allow the medical device to pass through the expandable sheath portion and the optional tapered end tip.

Referring again to FIG. 8, one or more portions of the expandable sheath portion 110 can also or alternatively include a breakable seam or one or more break points 700. The breakable seam or one or more break points 700 can be positioned along 5-100% (and all values and ranges therebetween) of the longitudinal length of the expandable sheath portion 110. The breakable seam or one or more break points 700 can be used to facilitate in maintaining the expandable sheath portion 110 in the unexpanded orientation. When the medical device is inserted through the expandable sheath portion 110 or a tool is inserted through the expandable sheath portion to break the break points 700 or breakable seam prior to inserting the medical device through the expandable sheath portion, the one or more break points 700 or breakable seam can be caused to be broken, thereby allowing the expandable sheath portion to expand to the expanded orientation and allow the medical device to pass through the expandable sheath portion.

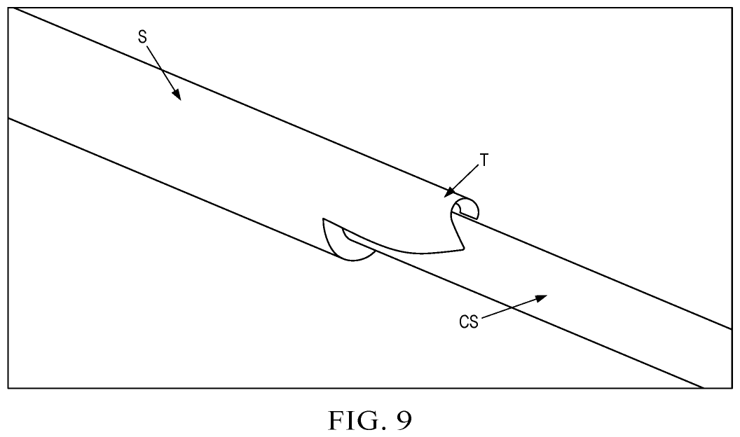

Referring now to FIG. 9, a photograph of a prior art sheath S is shown wherein a portion of a catheter shaft CS has been through the tip portion T of the sheath S resulting in the tip portion T of the expandable sheath S deflecting away from the catheter shaft CS. When the tip portion T of the expandable sheath S deflects away from the longitudinal axis of the expandable sheath S (i.e., a portion of the tip portion T of the expandable sheath S does not remain coaxial to the longitudinal axis of the expandable sheath S), the tip portion T can be exposed to/contact the vessel wall and movement of the expandable sheath S along the vessel wall can cause damage (dissection, perforation, etc.) to the vessel wall. In practice, when the tip portion T is expanded as the catheter shaft CS is passed through the end region of the expandable sheath S, there is a force that tries to retain its initial smaller outside diameter of the expandable sheath S. The force increases with the stiffness of the material of the tip portion T of the expandable sheath S. If the force is great enough, such force will cause a) the tip portion T of the expandable sheath S to move non-coaxial with the longitudinal axis of the expandable sheath S, which non-coaxial movement of the tip portion T can result in damage to the wall of the vessel, and/or b) cause the catheter shaft CS to move non-coaxial with the longitudinal axis of the expandable sheath S, which non-coaxial movement of the expandable sheath S can cause damage the wall of a vessel as the catheter shaft CS is moved through the expandable sheath S and into the vessel. When the longitudinal cut of the tip portion T is completely parallel to the central lumen of the expandable sheath S, as in many prior art expandable sheaths, the force to deflect the catheter shaft CS away from being coaxial with the longitudinal axis of the expandable sheath S is lower than the force on the tip portion T that tries to retain the smaller diameter of the tip region of the expandable sheath S, and therefore the catheter shaft CS is deflected away from being coaxial with the longitudinal axis of the expandable sheath S, and/or the tip region T of the expandable sheath S is caused to move away from being coaxial with the longitudinal axis of the expandable sheath S as illustrated in FIG. 9.

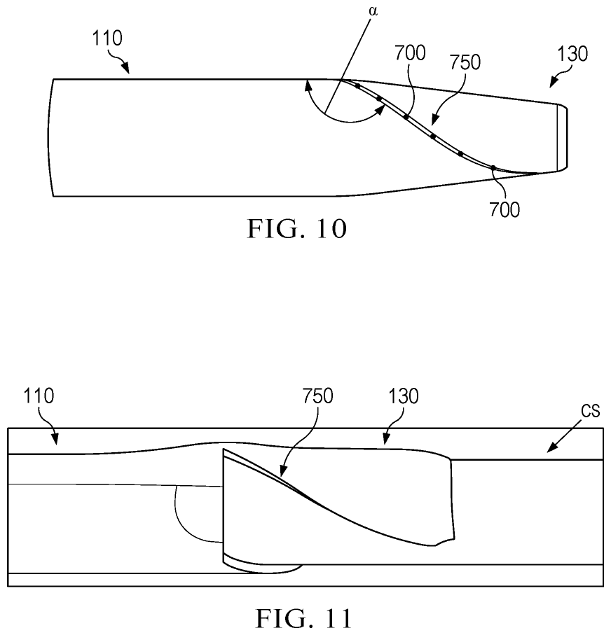

Referring now to FIGS. 10 and 11, there are illustrated two side views of a specially cut tip portion 130 of an expandable sheath portion 110 in accordance with the present disclosure that overcomes the issues of a) the catheter shaft being deflected away from being coaxial with the longitudinal axis of the expandable sheath as the catheter shaft is move out from the distal end of the expandable sheath, and/or b) the tip region of the expandable sheath being caused to move away from being coaxial with the longitudinal axis of the expandable sheath as the catheter shaft moves out from the distal end of the expandable sheath. In one non-limiting embodiment the distal end region of the expandable sheath portion 110 is absent a longitudinal cut that is completely parallel to the central lumen of the expandable sheath portion 110. In one non-limiting configuration, the end region of the expandable sheath portion 110 includes a spiral cut 750 as illustrated in FIGS. 10 and 11. The spiral cut 750 in the distal end region of the expandable sheath portion 110 facilitates in ensuring that there is always a portion of the cut tip portion 130 that wraps around the catheter shaft CS as the catheter shaft CS moves through the distal end region of the sheath, thereby keeping the catheter shaft CS in a coaxial configuration relative to the expandable sheath portion 110.

FIG. 11 illustrates that as the catheter shaft CS advances through the distal end region of the expandable sheath portion, the spiral cut 750 in the distal end region of the expandable sheath portion 110 enable the distal end region of the expandable sheath portion to wrap around the catheter shaft CS, thereby maintaining the catheter shaft CS in a coaxial orientation relative to the central lumen of the expandable sheath portion as the catheter shaft CS advances through the expandable sheath portion. Also, the expanded portion of the distal end region of the expandable sheath portion 110 also remains essentially or very close to the central axis of the lumen of the expandable sheath portion as the catheter shaft CS advances through the expandable sheath portion. As such, as the catheter shaft CS is advanced through the distal end region of the expandable sheath, the spiral cut 750 in the distal end region of the expandable sheath portion 110 inhibits or prevents damage to the inner walls of the vessels from the expanded regions of the distal end region of the expandable sheath portion and/or from the catheter shaft moving out of alignment with the longitudinal axis of the expandable sheath portion.

As illustrated in FIG. 10, the distal end region of the expandable sheath portion 110 is tapered end portion 130 that includes the spiral cut 750 in the tapered end portion 130. The spiral cut is illustrated as being limited to only a portion or all of the tapered end portion 130; however, this is not required.

In one non-limiting specific configuration, the spiral cut 750 in the distal end region of the expandable sheath portion 110 is cut at an angle a of at least 5° and less than 180° (and all values and ranges therebetween) from the axis that is parallel to the central lumen of the expandable sheath portion 110. As defined herein, a longitudinal cut would be 180° or parallel to the central lumen of the expandable sheath portion 110. In one non-limiting configuration the spiral cut in the distal end region of the sheath is cut at an angle of 105-160° (and all values and ranges therebetween). A small angle cut of the spiral cut 750 will result in more of the distal end region of the expandable sheath wrapping around the body of the catheter shaft CS. In another non-limiting configuration, the longitudinal length of the spiral cut 750 and/or the angle a of the spiral cut 750 is selected so that the a portion of the distal end region of the expandable sheath portion that includes the spiral cut 750 extends about 40-100% (and all values and ranges therebetween) of the circumference of the distal end region of the expandable sheath portion 110 that includes the spiral cut 750. As illustrated in FIGS. 10 and 11, the spiral cut 750 extends about 45-55% of the circumference of the distal end region of the expandable sheath portion 110 that includes the spiral cut 750. As can also be appreciated, the spiral cut 750 can extend more than one revolution about the circumference of the distal end region of the expandable sheath portion 110 that includes the spiral cut 750; however, this is not required. In another non-limiting configuration, the spiral cut 750 can extend along the longitudinal length of the expandable sheath a distance of at least 0.4 mm (e.g., 0.4-20 mm and all values and ranges therebetween) or 0.01-10% (and all values and ranges therebetween) of the longitudinal length of the expandable sheath portion 110. In another non-limiting configuration, the spiral cut 750 can extend along the longitudinal length of the expandable sheath a distance of 5-10 mm.

Referring again to FIG. 10, the spiral cut 750 can optionally include a breakable seam or one or more break points 700 to maintain the distal end region of the expandable sheath portion that includes the spiral cut 750 in a closed or unexpanded orientation. As discussed above, the a breakable seam or one or more break points 700 can be configured to break open to allow the spiral cut to open and allow the medical device MD, catheter shaft CS or other type of medical device to pass through the distal end region of the expandable sheath portion 110.

The optional tapered end portion 130 at the end region of the expandable sheath portion 110 can optionally be a separate component that is connected (e.g., adhesive connection, melted connection, friction connection, weld or soldered connection, etc.) to the distal end or distal end region of the main body of the expandable sheath portion 110. When the optional tapered end portion 130 is a separate component, the spiral cut 750, when used, can be exclusively formed in such optional separate tapered end portion 130, and optionally be absent from the main body of the expandable sheath portion 110; however, this is not required. When the optional tapered end portion 130 is a separate component from the main body of the expandable sheath portion 110, the optional tapered end portion 130 can form 0.1-20% (and all values and ranges therebetween) of the longitudinal length of the expandable sheath portion 110. Also, when the optional tapered end portion 130 is a separate component from the main body of the expandable sheath portion 110, the optional tapered end portion 130 can be formed of the same material or different material from the main body of the expandable sheath portion 110. In one non-limiting configuration, the optional tapered end portion 130 includes or is formed of thermoplastics (Pebax, Nylon, etc.) or metals (nitinol, stainless steel, etc.). In another and/or alternative non-limiting configuration, the optional tapered end portion 130 is formed of a different material from the main body of the expandable sheath portion 110. In another and/or alternative non-limiting configuration, the optional tapered end portion 130 is formed of a polymer material.

The expandable sheath 100 can optionally include one or more radiopaque markers or fillers. The radiopaque filler or marker, when used, can be located on the outer surface of the expandable sheath, embedded on one or more layers of the expandable sheath, and/or be located between on or more layers of the expandable sheath. The location of the one or more radiopaque markers or fillers on the expandable sheath 100 is non-limiting. Non-limiting materials that can be used as a radiopaque filler or marker include barium sulfite, bismuth trioxide, titanium dioxide, and/or bismuth subcarbonate.

The expandable sheath 100 in accordance with the present disclosure can be used a) to minimize trauma to a body passageway (e.g., blood vessel, etc.) by allowing for temporary expansion of a portion of the expandable sheath to accommodate a medical device and/or a delivery system for a medical device, and thereafter the expandable sheath is configured to return to its original diameter or close to its original diameter once the medical device and/or a portion of the delivery system passes through expandable sheath 100, b) to reduce the length of time a procedure takes, c) to reduce the risk of a longitudinal or radial body passageway tear, d) to reduce risk of plaque dislodgement in a body passageway, e) to reduce or eliminate the need for multiple insertions sheaths or other devices for the dilation of a body passageway, f) for many types of minimally invasive surgery, such as any surgery requiring introduction of a medical device (e.g., stent, prosthetic heart valve, grafts, etc.) into a body passageway (e.g., veins, arteries, esophagus, ducts of the biliary tree, intestine, urethra, fallopian tube, other endocrine or exocrine ducts, etc.).

The expandable sheath 100, in accordance with the present disclosure, can be used with various methods of introducing a medical device into a patient's vasculature. One such method comprises positioning an expandable sheath in a patient's vessel, passing a medical device through the expandable sheath 100, which causes a portion of the expandable sheath surrounding the device to expand and accommodate the profile of the medical device, and then retracting the expanded portion of the expandable sheath 100 to its original size or near original size after the medical device has partially or fully passed through the expandable sheath 100. In some methods, the expandable sheath 100 can be sutured to the patient's skin at the insertion site so that once the expandable sheath 100 is inserted the proper distance within the patient's vasculature, it does not move once the implantable medical device starts to travel through the expandable sheath 100.

The expandable sheath 100 in accordance with the present disclosure can be used with other delivery and minimally invasive surgical components, such as an introducer and loader. In one non-limiting embodiment, the expandable sheath 100 can be flushed to purge any air within the expandable sheath 100. An introducer can be inserted into the expandable sheath 100 and the introducer/sheath combination can be fully inserted into vasculature over a guiding device, such as a guidewire. Once the expandable sheath 100 and introducer are fully inserted into a patient's vasculature, the expandable sheath 100 can be optionally sutured in place at the insertion site. In this manner, the expandable sheath 100 can be substantially prevented from moving once positioned within the patient. The introducer can then be removed and a medical device, such as a transcatheter heart valve, can be inserted through the expandable sheath 100. Such methods can additionally comprise placing the heart valve in a crimped state on the distal end portion of an elongated delivery apparatus, and inserting the elongated delivery device with the crimped valve into and through the expandable sheath 100. Next, the delivery apparatus can be advanced through the patient's vasculature to the treatment site, where the valve can be implanted. Typically, the medical device has a greater outer diameter than the diameter of the expandable sheath 100 in its fully contracted orientation. The medical device can be advanced through the expandable sheath 100 towards the implantation site, and the expandable sheath 100 can locally expand to accommodate the medical device as the device passes through the expandable sheath 100. The radial force exerted by the medical device on the interior passageway or cavity of the expandable sheath 100 can be sufficient to locally expand the expandable sheath 100 to an expanded diameter (e.g., the fully expanded position or orientation) just in the area where the medical device is currently located or the complete sheath can be expanded along its longitudinal length. Once the medical device passes a particular location of the expandable sheath 100, the expandable sheath 100 can at least partially contract to a smaller diameter. Alternatively, the expandable sheath 100 can be configured to contract after the medical device has fully passed through the expandable sheath 100. Once the medical device is implanted, the expandable sheath 100 and any sutures holding it in place can be removed.

Expandable sheath 100 can be configured to expand to an expanded outer diameter that is from about 10% greater than the original unexpanded outer diameter to about 500% greater than the original unexpanded outer diameter.

Although example embodiments have been disclosed, a worker of ordinary skill in this art would recognize that certain modifications would come within the scope of the claims. For that reason, the following claims should be studied to determine their true scope and content.

It will thus be seen that the objects set forth above, among those made apparent from the preceding description, are efficiently attained, and since certain changes may be made in the constructions set forth without departing from the spirit and scope of the disclosure, it is intended that all matter contained in the above description and shown in the accompanying drawings shall be interpreted as illustrative and not in a limiting sense. The disclosure has been described with reference to preferred and alternate embodiments. Modifications and alterations will become apparent to those skilled in the art upon reading and understanding the detailed discussion of the disclosure provided herein. This disclosure is intended to include all such modifications and alterations insofar as they come within the scope of the present disclosure. It is also to be understood that the following claims are intended to cover all of the generic and specific features of the disclosure herein described and all statements of the scope of the disclosure which, as a matter of language, might be said to fall therebetween. The disclosure has been described with reference to the preferred embodiments. These and other modifications of the preferred embodiments, as well as other embodiments of the disclosure, will be obvious from the disclosure herein, whereby the foregoing descriptive matter is to be interpreted merely as illustrative of the disclosure and not as a limitation. It is intended to include all such modifications and alterations insofar as they come within the scope of the appended claims.

To aid the Patent Office and any readers of this application and any resulting patent in interpreting the claims appended hereto, applicants do not intend any of the appended claims or claim elements to invoke 35 U.S.C. 112(f) unless the words “means for” or “step for” are explicitly used in the particular claim. The listing of claims will replace all prior versions, and listings of claims in the application:

1. An expandable sheath that provides access to a body passageway to facilitate in delivering a medical device into the body passageway; said expandable sheath includes an expandable sheath portion that is expandable from an unexpanded orientation having a first cross-sectional area to an expanded orientation having a second cross-sectional area; said second cross-sectional area is greater than said first cross-sectional area; said expandable sheath portion includes a tubular structure along 80-100% of said longitudinal length of said expandable sheath portion; said tubular structure is at least partially formed of a first polymer; said expandable sheath portion includes an internal cavity that extends at least 25% of a longitudinal length of said expandable sheath portion; said tubular structure includes a thick portion and a thin portion; an average thickness of said thick portion is greater than an average thickness of said thin portion; said thick portion includes first and second ends; said thin portion is connected to and/or overlies said first and second ends; a cross-sectional shape of said expandable sheath portion that is normal to a longitudinal axis of said tubular structure includes A) said thick portion that is positioned about 30-90% of a circumference of said internal cavity of said expandable sheath portion when said expandable sheath portion is in said expanded orientation, and B) said thin portion that is positioned about 10-70% of said circumference of said internal cavity of said expandable sheath portion when said expandable sheath portion is in said expanded orientation; combined portions of said thick portion and said thin portion fully encircle said internal cavity; said cross-sectional shape of said tubular structure is constant along 25-100% of said longitudinal length of said tubular structure when said expandable sheath portion is in said expanded orientation; a first end portion and a second end portion of said thick portion overlie one another such that a portion of an outer perimeter of said tubular structure includes overlying said first and second end portions when said when said expandable sheath portion is in said unexpanded orientation; said first end portion and said second end portion of said tubular structure are spaced apart from one another when said when said expandable sheath portion is in said expanded orientation; and
wherein said expandable sheath portion is absent a frame; and
wherein 50-100% of a longitudinal length of said thin portion is positioned between overlapping regions of said first end portion and said second end portion when said expandable sheath portion is in said unexpanded orientation;
wherein 30-99.9% of a circumference of said second polymer portion is positioned between overlapping regions of said first polymer portion when said expandable portion of said sheath is in said unexpanded orientation; and
wherein said expandable sheath portion is configured to expand to said second cross-sectional area during movement of the medical device through said internal cavity of said tubular structure; and
wherein said expandable sheath portion is configured to contract from said second cross-sectional area after the medical device partially or fully passes through said internal cavity of said tubular structure.

2. The expandable sheath as defined in claim 1, wherein said thick portion and said thin portion are formed by said first polymer; and wherein there is no connection mechanism used between said thick portion and said thin portion.

3. The expandable sheath as defined in claim 1, wherein said thick portion is formed by said first polymer and said thin portion is formed by a second polymer; said thick portion includes first and second ends; said thin portion includes first and second ends; said first ends of said thick and thin portions are connected together; said second ends of said thick and thin portions are connected together; said thick portion is connected to said thin portion by one or more connection mechanisms.

4. The expandable sheath as defined in claim 1, wherein said thick portion is formed by said first polymer and said thin portion is formed by a second polymer; said thick portion includes first and second ends; said thin portion is formed in a continuous layer; said thin portion is connected to one or more portions of an outer surface of said thick portion; said thin portion overlies 50-100% of said outer surface of said thick portion; a portion of said thin portion spans a gap between said first and second ends of said thick portions when said expandable sheath portion is in said expanded orientation; said thin portion is positioned along 50-100% of a longitudinal length of said thick portion.

5. The expandable sheath as defined in claim 1, wherein an average durometer of material used to form said thick portion is greater than an average durometer of material used to form said thin portion.

6. The expandable sheath as defined in claim 1, wherein 80-100% of a circumferential length of said thick portion of said tubular structure is flexible, but is not foldable; 5-100% of a circumferential length of said thin portion is both flexible and foldable.

7. The expandable sheath as defined in claim 1, wherein said expandable sheath portion includes an internal liner; said internal liner is positioned in said internal cavity of said tubular structure along 50-100% of said longitudinal length of said tubular structure; said internal liner is formed of a different material from said material used to form said tubular structure.

8. The expandable sheath as defined in claim 7, wherein said internal liner has a melting point that is greater than said material used to form said tubular structure.

9. The expandable sheath as defined in claim 7, wherein said internal liner has a static coefficient of friction that is less than a static coefficient of friction of said material used to form said tubular structure.

10. The expandable sheath as defined in claim 1, further including a hub connection portion; a distal end of said hub connection portion is connected to a proximal end of said expandable sheath portion; said hub connection portion is formed of a material that a) has a higher durometer than a durometer of said thick portion of said tubular structure, and/or b) is less flexible material than said material used to form said thick portion of said tubular structure.

11. The expandable sheath as defined in claim 1, wherein a distal end region of said expandable sheath portion includes a spiral cut.

12. The expandable sheath as defined in claim 1, wherein a distal end region of said expandable sheath portion includes a taper region.

13. The expandable sheath as defined in claim 12, wherein said distal end region is a separate component from said tubular structure; said distal end region is connected to said tubular structure by one or more connection mechanisms that include an adhesive, a heat created connection, a melted connection, a pressure connection, and/or a mechanical connection.

14. The expandable sheath as defined in claim 12, wherein a distal end region is formed of a different material from said tubular structure.

15-30. (canceled)

31. An expandable sheath that provides access to a body passageway to facilitate in delivering a medical device into the body passageway; said expandable sheath includes an expandable sheath portion that is expandable from an unexpanded orientation having a first cross-sectional area to an expanded orientation having a second cross-sectional area; said second cross-sectional area is greater than said first cross-sectional area; said expandable sheath portion includes a tubular structure along 80-100% of said longitudinal length of said expandable sheath portion;
said tubular structure is at least partially formed of a first polymer; said expandable sheath portion includes an internal cavity that extends at least 25% of a longitudinal length of said expandable sheath portion; said tubular structure includes a thick portion and a thin portion; an average thickness of said thick portion is greater than an average thickness of said thin portion; said thick portion includes first and second ends; said first end of said thin portion terminates at said first end of said thick portion; said second end of said thin portion terminates at said second end of said thick portion; said thick portion and said thin portion are formed by said first polymer; a cross-sectional shape of said expandable sheath portion that is normal to a longitudinal axis of said tubular structure includes A) said thick portion that is positioned about 30-90% of a circumference of said internal cavity of said expandable sheath portion when said expandable sheath portion is in said expanded orientation, and B) said thin portion that is positioned about 10-70% of said circumference of said internal cavity of said expandable sheath portion when said expandable sheath portion is in said expanded orientation; combined portions of said thick portion and said thin portion fully encircle said internal cavity along at least 30% of said longitudinal length of said internal cavity when said expandable sheath portion is in said expanded orientation; said cross-sectional shape of said tubular structure is constant along 25-100% of said longitudinal length of said tubular structure when said expandable sheath portion is in said expanded orientation; a first end portion and a second end portion of said thick portion overlie one another such that a portion of an outer perimeter of said tubular structure includes overlying said first and second end portions when said when said expandable sheath portion is in said unexpanded orientation; said first end portion and said second end portion of said tubular structure are spaced apart from one another when said when said expandable sheath portion is in said expanded orientation; and
wherein said expandable sheath portion is absent a frame; and
wherein 50-100% of a longitudinal length of said thin portion is positioned between overlapping regions of said first end portion and said second end portion when said expandable sheath portion is in said unexpanded orientation;
wherein 30-99.9% of a circumferential length said thin portion is positioned between overlapping regions of said first polymer portion when said expandable portion of said sheath is in said unexpanded orientation; and
wherein said expandable sheath portion is configured to expand to said second cross-sectional area during movement of the medical device through said internal cavity of said tubular structure; and
wherein said expandable sheath portion is configured to contract from said second cross-sectional area after the medical device partially or fully passes through said internal cavity of said tubular structure.

32. The expandable sheath as defined in claim 31, wherein there is no connection mechanism used between said thick portion and said thin portion.

33. The expandable sheath as defined in claim 31, wherein an average durometer of material used to form said thick portion is greater than an average durometer of material used to form said thin portion; 80-100% of a circumferential length of said thick portion of said tubular structure is flexible, but is not foldable; 5-100% of a circumferential length of said thin portion is both flexible and foldable.

34. The expandable sheath as defined in claim 32, wherein an average durometer of material used to form said thick portion is greater than an average durometer of material used to form said thin portion; 80-100% of a circumferential length of said thick portion of said tubular structure is flexible, but is not foldable; 5-100% of a circumferential length of said thin portion is both flexible and foldable.

35. The expandable sheath as defined in claim 31, wherein said expandable sheath portion includes an internal liner; said internal liner is positioned in said internal cavity of said tubular structure along 50-100% of said longitudinal length of said tubular structure; said internal liner is formed of a different material from said material used to form said tubular structure.

36. The expandable sheath as defined in claim 34, wherein said expandable sheath portion includes an internal liner; said internal liner is positioned in said internal cavity of said tubular structure along 50-100% of said longitudinal length of said tubular structure; said internal liner is formed of a different material from said material used to form said tubular structure.

37. The expandable sheath as defined in claim 35, wherein said internal liner has a melting point that is greater than said material used to form said tubular structure.

38. The expandable sheath as defined in claim 36, wherein said internal liner has a melting point that is greater than said material used to form said tubular structure.

39. The expandable sheath as defined in claim 35, wherein said internal liner has a static coefficient of friction that is less than a static coefficient of friction of said material used to form said tubular structure.

40. The expandable sheath as defined in claim 38, wherein said internal liner has a static coefficient of friction that is less than a static coefficient of friction of said material used to form said tubular structure.

41. The expandable sheath as defined in claim 31, further including a hub connection portion; a distal end of said hub connection portion is connected to a proximal end of said expandable sheath portion; said hub connection portion is formed of a material that a) has a higher durometer than a durometer of said thick portion of said tubular structure, and/or b) is less flexible material than said material used to form said thick portion of said tubular structure.

42. The expandable sheath as defined in claim 40, further including a hub connection portion; a distal end of said hub connection portion is connected to a proximal end of said expandable sheath portion; said hub connection portion is formed of a material that a) has a higher durometer than a durometer of said thick portion of said tubular structure, and/or b) is less flexible material than said material used to form said thick portion of said tubular structure.

43. The expandable sheath as defined in claim 31, wherein a distal end region of said expandable sheath portion includes a spiral cut.

44. The expandable sheath as defined in claim 42, wherein a distal end region of said expandable sheath portion includes a spiral cut.

45. The expandable sheath as defined in claim 31, wherein a distal end region of said expandable sheath portion includes a taper region.

46. The expandable sheath as defined in claim 44, wherein a distal end region of said expandable sheath portion includes a taper region.

47. The expandable sheath as defined in claim 31, wherein a distal end region is a separate component from said tubular structure; said distal end region is connected to said tubular structure by one or more connection mechanisms that include an adhesive, a heat created connection, a melted connection, a pressure connection, and/or a mechanical connection; a distal end region is formed of a different material from said tubular structure.

48. The expandable sheath as defined in claim 46, wherein a distal end region is a separate component from said tubular structure; said distal end region is connected to said tubular structure by one or more connection mechanisms that include an adhesive, a heat created connection, a melted connection, a pressure connection, and/or a mechanical connection; a distal end region is formed of a different material from said tubular structure.
